(19) United States
(12) Patent Application Publication
(10) Pub. No.: US 2020/0002303 A1
(43) Pub. Date: Jan. 2, 2020

(54) C-GLYCOSIDE COMPOUNDS USEFUL FOR TREATING DISEASE

(71) Applicant: Fimbrion Therapeutics, Inc., St Louis, MO (US)

(72) Inventors: James W. JANETKA, St. Louis, MO (US); Laurel MYDOCK-MCGRANE, St. Louis, MO (US)

(21) Appl. No.: 16/084,177

(22) PCT Filed: Mar. 11, 2017
(86) PCT No.: PCT/US17/21983

§ 371 Date: Sep. 11, 2018

Related U.S. Application Data
(60) Provisional application No. 62/307,078, filed on Mar. 11, 2016.

(51) Int. Cl. C07D 309/10; C07D 413/10; C07D 471/04

(52) U.S. Cl. CPC C07D 309/10; A61K 45/06; C07D 471/04; C07D 413/10

(57) ABSTRACT

The present invention relates to mannoside derivative compounds useful as inhibitors of FimH and methods for the treatment or prevention of urinary tract infection.

This application claims priority to U.S. provisional application No. 62/307,078 filed Mar. 11, 2016, the disclosure of which is incorporated by reference herein in its entirety.

Disclosed herein are new C-mannoside compounds and compositions and their application as pharmaceuticals for the treatment of disease. Methods for the inhibition of FimH function in a human or animal subject are also provided for the treatment and prevention of diseases such as urinary tract infection and Crohn's Disease.

Disclosed herein is a compound of Formula (I):

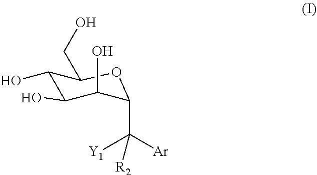

where:
Ar is aryl or heteroaryl;
where:
   each aryl and heteroaryl as defined for Ar is substituted with W and one or two Z groups;
   where:
      Z is lower alkyl, lower haloalkyl, NO₂, CF₃, cyclopropyl, lower alkoxy, halo, hydroxyl, and amino;
      where:
         amino as defined for Z is optionally substituted with one or two lower alkyl,
      W is aryl, heteroaryl, or azide;
         where:
         aryl or heteroaryl as defined for W is substituted with one or more substituents selected from R₁₁, H, boronic acid, boronic acid pinacol ester, alkyl, OTf, hydroxyl, amino optionally substituted with one or two alkyl or aryl groups, azide, alkyne, —SO₂Aryl; —C(O)OR₅, C(O)NR₈R₉, halo, OCF₃, alkenyl, alkynyl, haloalkyl, CN, alkoxy, NHSO₂R₆, NHSO₂NHR₆, NHCOR₆, NHCONHR₆, and cycloalkyl, heterocycloalkyl, aryl, aryloxy, aralkyl, and heteroaryl any of which may be optionally substituted with one or more alkyl, hydroxyl, oxo, CN, and NR₈R₉,
         where:
         each R₅ and R₆ independently is hydrogen, alkyl, cycloalkyl, heterocycloalkyl, aryl, heteroaryl, aralkyl and heteroaralkyl;
         each R₈ and R₉ is independently hydrogen, C₁-C₆ alkyl, aryl, heteroaryl, aralkyl, and heteroaralkyl; or
         R₈ and R₉ taken together form a heterocycloalkyl;
         R₁₁ is halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, NHSO₂R₁₂, NHSO₂NHR₁₂, NHCOR₁₂, NHCONHR₁₂, CONHR₁₂, CONR₁₂ₐR_{12b}, hydroxy, and OCF₃;
         where:
         each R₁₂, R₁₂ₐ and R_{12b} independently is selected from hydrogen, C₁-C₆ alkyl, aryl, heteroaryl, aralkyl and heteroaralkyl;
each Y₁ and Y₂ independently is selected from H, hydroxyl, lower alkoxy or amino;
where:
   each amino as defined for each Y₁ and Y₂ is optionally substituted with one or two lower alkyl, cyano, azide, nitro, haloalkyl, halo, haloalkoxy, and acetyl; provided that:
   the compound of Formula (I) is not:
3-[4-[(R)-hydroxy-[(2R,3S,4S,5S,6R)-3,4,5-trihydroxy-6-(hydroxymethyl)tetrahydropyran-2-yl]methyl]-3-methyl-phenyl]-N-methylbenzamide,
3-[4-[(S)-hydroxy-[2R,3S,4S,5S,6R)-3,4,5-trihydroxy-6-(hydroxymethyl)tetrahydropyran-2-yl]methyl]-3-methyl-phenyl]-N-methylbenzamide,
N-methyl-3-[3-methyl-4-[[(2R,3S,4R,5S,6R)-3,4,5-trihydroxy-6-(hydroxy-methyl)tetrahydro-pyran-2-yl]methyl]phenyl]benzamide,
4′-((R)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide,
N-methyl-4′-(((2R,3S,4R,5S,6R)-3,4,5-trihydroxy-6-(hydroxymethyl)tetrahydro-2H-pyran-2-yl)methyl)biphenyl-3-carboxamide,
4′-((S)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide,
N,3′-dimethyl-4′-(((2R,3S,4R,5S,6R)-3,4,5-trihydroxy-6-(hydroxymethyl)tetrahydro-2H-pyran-2-yl)methyl)biphenyl-3-carboxamide,
N-methyl-3′-(trifluoromethyl)-4′-(((2R,3S,4R,5S,6R)-3,4,5-trihydroxy-6-(hydroxymethyl)tetrahydro-2H-pyran-2-yl)methyl)biphenyl-3-carboxamide,
3′-chloro-N-methyl-4′-(((2R,3S,4R,5S,6R)-3,4,5-trihydroxy-6-(hydroxymethyl)tetrahydro-2H-pyran-2-yl)methyl)biphenyl-3-carboxamide,
3′-fluoro-N-methyl-4′-(((2R,3S,4R,5S,6R)-3,4,5-trihydroxy-6-(hydroxymethyl)tetrahydro-2H-pyran-2-yl)methyl)biphenyl-3-carboxamide,
3′-methoxy-N-methyl-4′-(((2R,3 S,4R,5S,6R)-3,4,5-trihydroxy-6-(hydroxymethyl)tetrahydro-2H-pyran-2-yl)methyl)biphenyl-3-carboxamide,
N³,N⁵-dimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide,
N³,N⁵,3′-trimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide,
N³,N⁵-dimethyl-3′-(trifluoromethyl)-4′-(((2R,3S,4R,5S,6R)-3,4,5-trihydroxy-6-(hydroxymethyl)tetrahydro-2H-pyran-2-yl)methyl)biphenyl-3,5-dicarboxamide,
3′-chloro-N³,N⁵-dimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide,
3′-fluoro-N³,N⁵-dimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide,
3′-methoxy-N³,N⁵-dimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide.

In an embodiment, R₁₁ is chosen from heterocyclyl and heteroaryl.

In an embodiment, Y₁ and Y₂ are not both H.

Certain compounds disclosed herein may possess useful FimH and type 1 pili inhibiting function, and may be used in the treatment or prophylaxis of a disease or condition in which FimH plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for binding to and inhibiting FimH function. Other embodiments provide methods for treating a FimH-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of FimH function. Certain embodiments also provide for the synthesis of key intermediates, as well as novel methods for C-glycoside synthesis.

In certain embodiments, the compounds have Formula (II):

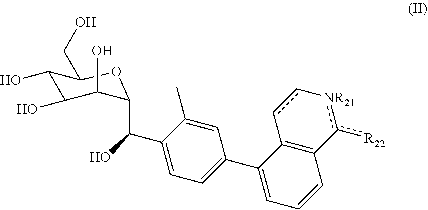

where:
   “----” represents a single or double bond;
   R₂₁ is null, hydrogen or lower alkyl;
   R₂₂ is hydrogen, alkyl, hydroxyl, O or NR₂₈R₂₉;
   where:
      each R₂₈ and R₂₉ independently is hydrogen, C₁-C₆ alkyl, aryl, heteroaryl, aralkyl or heteroaralkyl; or
      R₂₈ and R₂₉ taken together form a heterocycloalkyl; or
   a pharmaceutically acceptable salt thereof.

In certain embodiments, the compounds have Formula (III):

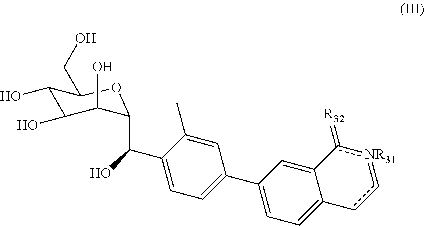

where:
   “----” represents a single or double bond;
   R₃₁ is null, hydrogen or lower alkyl;
   R₃₂ is hydrogen, alkyl, hydroxyl, O or NR₃₈R₃₉;
   where:
      each R₃₈ and R₃₉ independently is hydrogen, C₁-C₆ alkyl, aryl, heteroaryl, aralkyl, and heteroaralkyl; or
      R₃₈ and R₃₉ taken together form a heterocycloalkyl; or
a pharmaceutically acceptable salt thereof.

In certain further embodiments, the compounds have Formula (IV):

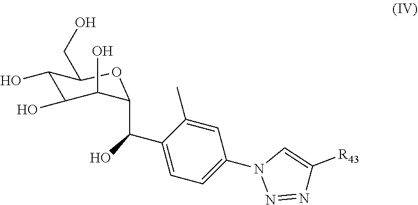

where:
   R₄₃ is alkyl, cycloalkyl, heterocycloalkyl, aryl or heteroaryl;
      where:
         each alkyl, cycloalkyl, heterocycloalkyl, aryl or heteroaryl as defined for R₄₃ is optionally substituted with one or more substituents selected from hydrogen, halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, COOR₄₄, NHSO₂R₄₄, NHSO₂NHR₄₄, NHCOR₄₄, NHCONHR₄₄, CONHR₄₄, CONR₄₄ₐR_{44b}, hydroxy, or OCF₃;
         where:
         each R₄₄, R₄₄ₐ and R_{44b} independently is selected from hydrogen, C₁-C₆ alkyl, aryl, heteroaryl, aralkyl or heteroaralkyl; or
         a pharmaceutically acceptable salt thereof.

In certain further embodiments, the compounds have structural Formula V:

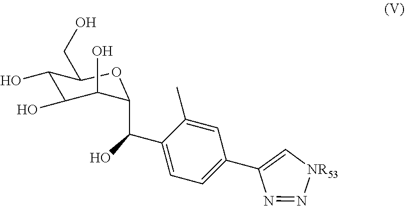

where:
   R₅₃ is null, hydrogen or lower alkyl; or
a pharmaceutically acceptable salt thereof.

In certain further embodiments, the compounds have Formula (VI):

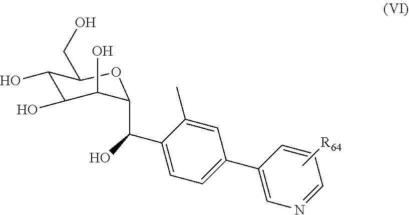

where:
   R₆₄ is —C(O)OR₆₅, C(O)NR₆₈R₆₉, halo, hydroxy, OCF₃, alkyl, alkenyl, alkynyl, cycloalkyl, haloalkyl, aralkyl, heterocycloalkyl, CN, alkoxy, amino, alkylamino, dialkylamino, NHSO₂R₆₆, NHSO₂NHR₆₆, NHCOR₆₆, NHCONHR₆₆; or aryl or heteroaryl either of which may be optionally substituted with halo, hydroxy, OCF₃, alkyl, alkenyl, alkynyl, haloalkyl, CN, alkoxy, alkylamino, dialkylamino, NHSO₂R₆₆, NHSO₂NHR₆₆, NHCOR₆₆, or NHCONHR₆₆;
   where:
      R₆₅ is hydrogen, alkyl, cycloalkyl, heterocycloalkyl, aryl or heteroaryl;
      each R₆₈ and R₆₉ are each independently chosen from hydrogen, C₁-C₆ alkyl, aryl, heteroaryl, aralkyl, and heteroaralkyl, or R₆₈ and R₆₉ taken together form a heterocycloalkyl; and
      R₆₆ is hydrogen, C₁-C₆ alkyl, aryl, heteroaryl, aralkyl and heteroaralkyl; or
   a pharmaceutically acceptable salt thereof.

In certain further embodiments, the compounds have Formula (VII):

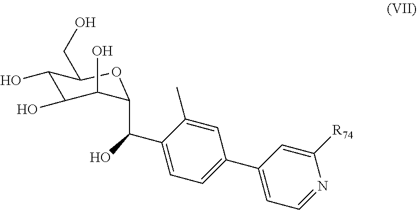

where:
   R₇₄ is —C(O)OR₇₅, C(O)NR₇₈R₇₉, halo, hydroxy, OCF₃, alkyl, alkenyl, alkynyl, cycloalkyl, haloalkyl, aralkyl, heterocycloalkyl, CN, alkoxy, alkylamino, dialkylamino, NHSO₂R₇₇, NHSO₂NHR₇₇, NHCOR₇₇, NHCONHR₇₇; or aryl or heteroaryl either of which optionally is substituted with halo, hydroxy, OCF₃, alkyl, alkenyl, alkynyl, haloalkyl, CN, alkoxy, alkylamino, dialkylamino, NHSO₂R₇₇, NHSO₂NHR₇₇, NHCOR₇₇ or NHCONHR₇₇;
   where:
      R₇₅ is hydrogen, alkyl, cycloalkyl, heterocycloalkyl, aryl or heteroaryl;
      R₇₈ and R₇₉ are each independently chosen from hydrogen, C₁-C₆ alkyl, aryl, heteroaryl, aralkyl, and heteroaralkyl;
      R₇₈ and R₇₉ taken together form a heterocycloalkyl; and
      R₇₇ is hydrogen, C₁-C₆ alkyl, aryl, heteroaryl, aralkyl or heteroaralkyl;
or a pharmaceutically acceptable salt thereof.

In certain further embodiments, the compounds have Formula (VIII):

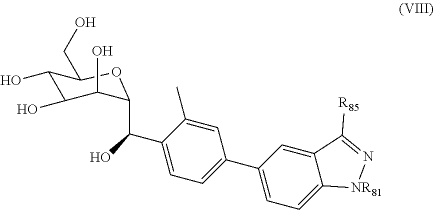

where:
   R₈₁ is from null, hydrogen, and lower alkyl;
   R₈₅ is from hydrogen, alkyl, NR₈₈R₈₉, aryl, heteroaryl, cycloalkyl, and heterocycloalkyl, any of which may be optionally substituted;
      where
         each R₈₈ and R₈₉ independently is hydrogen, C₁-C₆ alkyl, aryl, heteroaryl, aralkyl, or heteroaralkyl, or
         R₈₈ and R₈₉ taken together form a heterocycloalkyl; or
   a pharmaceutically acceptable salt thereof.

In certain further embodiments, the compounds have structural Formula IX:

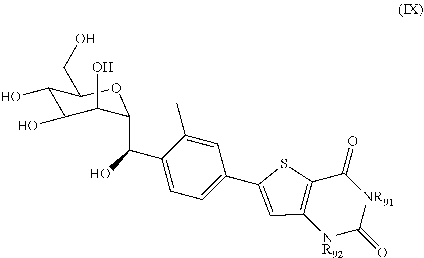

where:
   each R₉₁ and R₉₂ independently is hydrogen or lower alkyl; or
a pharmaceutically acceptable salt thereof.

In certain further embodiments, the compounds have Formula (X):

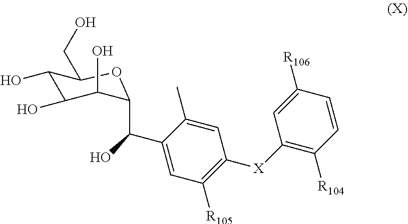

where:
   R₁₀₆ is from cyano, C(O)NR₁₀₉R₁₁₀, NR₁₀₉R₁₁₀, —SO₂NR₁₁₁R₁₁₂, NHC(O)NR₁₀₉R₁₁₀, nitro, hydroxyl, halo, and heteroaryl;
   each R₁₀₉ and R₁₁₀ independently is hydrogen, C₁-C₆ alkyl, aryl, heteroaryl, aralkyl, and heteroaralkyl, or taken together, R₁₀₉ and R₁₁₀ may form a heterocycloalkyl; and
   each R₁₁₁ and R₁₁₂ independently is H, C₃-C₇-cycloalkyl; C₂-C₆-alkyl, aryl, or heteroaryl; or
   R₁₁₁ and R₁₁₂ together with the atom to which they are attached form a C₃-C₇-heterocycloalkyl or heteroaryl;
   each R₁₀₄ and R₁₀₅ independently is hydrogen or nitro; and
   X is O, NH, or SO₂; or
a pharmaceutically acceptable salt thereof.

In certain further embodiments, the compounds have Formula (XI):

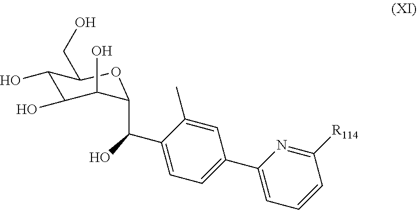

where:
   R₁₁₄ is —C(O)OR₁₁₅, C(O)NR₁₁₈R₁₁₉, halo, hydroxy, OCF₃, alkyl, alkenyl, alkynyl, cycloalkyl, haloalkyl, aralkyl, heterocycloalkyl, CN, alkoxy, alkylamino, dialkylamino, NHSO₂R₁₁₇, NHSO₂NHR₁₁₇, NHCOR₁₁₇, NHCONHR₁₁₇, and aryl and heteroaryl which may be optionally substituted with halo, hydroxy, OCF₃, alkyl, alkenyl, alkynyl, haloalkyl, CN, alkoxy, alkylamino, dialkylamino, NHSO₂R₁₁₇, NHSO₂NHR₁₁₇, NHCOR₁₁₇, or NHCONHR₁₁₇;
   R₁₁₅ is chosen from hydrogen, alkyl, cycloalkyl, heterocycloalkyl, aryl, heteroaryl;
   R₁₁₈ and R₁₁₉ are each independently chosen from hydrogen, C₁-C₆ alkyl, aryl, heteroaryl, aralkyl, and heteroaralkyl, or taken together, R₁₁₈ and R₁₁₉ may form a heterocycloalkyl; and
   R₁₁₇ is hydrogen, C₁-C₆ alkyl, aryl, heteroaryl, aralkyl and heteroaralkyl; or
a pharmaceutically acceptable salt thereof.

In certain embodiments of the present invention, compounds have structural Formula XII:

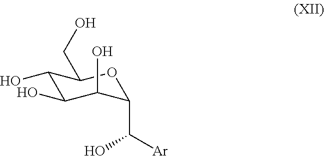

where:
Ar is aryl or heteroaryl;
where:
   each aryl and heteroaryl as defined for Ar is substituted with W and one or two Z groups;
   where:
      Z is lower alkyl, lower haloalkyl, NO₂, CF₃, cyclopropyl, lower alkoxy, halo, hydroxyl, and amino;
      where:
         amino as defined for Z is optionally substituted with one or two lower alkyl,
      W is aryl, heteroaryl, or azide;
         where:
         aryl or heteroaryl as defined for W is substituted with one or more substituents selected from R₁₁, H, boronic acid, boronic acid pinacol ester, alkyl, OTf, hydroxyl, amino optionally substituted with one or two alkyl or aryl groups, azide, alkyne, —SO₂Aryl; —C(O)OR₅, C(O)NR₈R₉, halo, OCF₃, alkenyl, alkynyl, haloalkyl, CN, alkoxy, NHSO₂R₆, NHSO₂NHR₆, NHCOR₆, NHCONHR₆, and cycloalkyl, heterocycloalkyl, aryl, aryloxy, aralkyl, and heteroaryl any of which may be optionally substituted with one or more alkyl, hydroxyl, oxo, CN, and NR₈R₉,
         where:
         each R₅ and R₆ independently is hydrogen, alkyl, cycloalkyl, heterocycloalkyl, aryl, heteroaryl, aralkyl and heteroaralkyl;
         each R₈ and R₉ is independently hydrogen, C₁-C₆ alkyl, aryl, heteroaryl, aralkyl, and heteroaralkyl; or
         R₈ and R₉ taken together form a heterocycloalkyl;
         R₁₁ is halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, NHSO₂R₁₂, NHSO₂NHR₁₂, NHCOR₁₂, NHCONHR₁₂, CONHR₁₂, CONR₁₂ₐR_{12b}, hydroxy, and OCF₃;
         where:
         each R₁₂, R₁₂ₐ and R_{12b} independently is selected from hydrogen, C₁-C₆ alkyl, aryl, heteroaryl, aralkyl and heteroaralkyl;
provided that:
   the compound is not:
3-[4-[(S)-hydroxy-[2R,3S,4S,5S,6R)-3,4,5-trihydroxy-6-(hydroxymethyl)tetrahydropyran-2-yl]methyl]-3-methyl-phenyl]-N-methylbenzamide, or
4′-((S)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide,
   or an ester or pharmaceutically acceptable salt thereof.

In certain embodiments, said compounds have Formula (XIII):

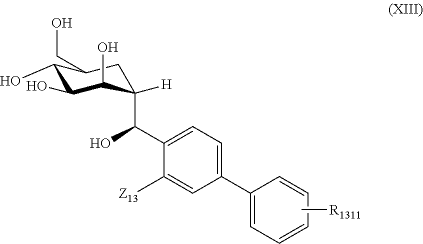

where:
R₁₃₁₁ is chosen from halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, NHSO₂R₁₃₁₂, NHSO₂NHR₁₃₁₂, NHCOR₁₃₁₂, NHCONHR₁₃₁₂, CONHR₁₃₁₂, CONR₁₃₁₂ₐR_{1312b}, hydroxy, and OCF₃;
   where
      R₁₃₁₂, R₁₃₁₂ₐ and R_{1312b} are independently chosen from hydrogen, C₁-C₆ alkyl, aryl, heteroaryl, aralkyl and heteroaralkyl; and
Z₁₃ is chosen from lower alkyl, lower haloalkyl, NO₂, CF₃, cyclopropyl, lower alkoxy, halo, hydroxyl, and amino optionally substituted with one or two lower alkyl with the proviso that the compound is not
3-[4-[(R)-hydroxy-[(2R,3S,4S,5S,6R)-3,4,5-trihydroxy-6-(hydroxymethyl)tetrahydropyran-2-yl]methyl]-3-methyl-phenyl]-N-methylbenzamide.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

Also provided is a compound chosen from the Examples disclosed herein.

The present invention also relates to a method of inhibiting at least one FimH function comprising the step of contacting FimH with a compound as described herein. The cell phenotype, cell proliferation, activity of FimH, change in biochemical output produced by active FimH, expression of FimH, or binding of FimH with a natural binding partner may be monitored. Such methods may be modes of treatment of disease, biological assays, cellular assays, biochemical assays, or the like.

Also provided herein is a method of treatment or prevention of a FimH-mediated disease comprising the administration of a therapeutically effective amount of a compound as disclosed herein, or a salt thereof, to a patient in need thereof.

In an embodiment, said disease is an antibiotic-resistant bacterial infection.

In certain embodiments, said disease is chosen from urinary tract infection.

In certain embodiments, said disease is chosen from Crohn's Disease.

In certain embodiments, said disease is chosen from Inflammatory Bowel Disease.

In an embodiment, said urinary tract infection is chronic or recurrant.

Also provided herein is a compound as disclosed herein for use as a medicament.

Also provided herein is a compound as disclosed herein for use as a medicament for the treatment of a FimH-mediated disease.

Also provided is the use of a compound as disclosed herein as a medicament.

Also provided is the use of a compound as disclosed herein as a medicament for the treatment of a FimH-mediated disease.

Also provided is a compound as disclosed herein for use in the manufacture of a medicament for the treatment of a FimH-mediated disease.

Also provided is the use of a compound as disclosed herein for the treatment of a FimH-mediated disease.

Also provided herein is a method of inhibition of FimH function comprising contacting FimH with a compound as disclosed herein, or a salt thereof.

In certain embodiments, the FimH-mediated disease is chosen from urinary tract infection.

In certain embodiments, the FimH-mediated disease is chosen from Crohn's Disease.

In certain embodiments, the FimH-mediated disease is chosen from Inflammatory Bowel Disease

Also provided is a method of inhibition of FimH-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral (PO) administration.

In certain embodiments, the oral pharmaceutical composition is chosen from a tablet and a capsule.

Also provided is a method of treatment of a FimH-mediated disease comprising the administration of:
a. a therapeutically effective amount of a compound of Formula (I) according to Claim 1; and
b. another therapeutic agent.

### Terms

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C═C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC (O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C (O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC (O)NRR′, group— with R and R′ as defined herein.

The term “N-carbamyl” as used herein, alone or in combination, refers to a ROC (O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom (s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N (OH) and ═N—O—.

The phrase “in the main chain” refers to the longest contiguous or adjacent chain of carbon atoms starting at the point of attachment of a group to the compounds of any one of the formulas disclosed herein.

The term “independently” means that where more than one substituent is selected from a number of possible substituents, those substituents may be the same or different.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC (S)NR′— group, with R and R′ as defined herein.

The term “O-thiocarbamyl” refers to a —OC (S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C (O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The compounds according to Formulas (I) to (XIII), respectively, or a pharmaceutically acceptable salt thereof of the present invention may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formulas (I) to (XIII), respectively, or a pharmaceutically acceptable salt thereof, or in any chemical structure illustrated herein, is not specified the structure is intended to encompass all individual stereoisomers and all mixtures thereof. Thus, compounds according to Formulas (I) to (XIII), respectively, or a pharmaceutically acceptable salt thereof, containing one or more chiral center may be used as racemic mixtures, enantiomerically enriched mixtures, or as enantiomerically pure individual stereoisomers.

Individual stereoisomers of a compound according to Formulas (I) to (XIII), respectively, or a pharmaceutically acceptable salt thereof, which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation. When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

Because of their potential use in medicine, the salts of the compounds of Formulas (I) to (XIII), respectively, are preferably pharmaceutically acceptable salts. Suitable pharmaceutically acceptable salts include those described by Berge, Bighley and Monkhouse J. Pharm. Sci (1977) 66, pp 1-19.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, g-hydroxybutyrates, glycollates, tartrates mandelates, and sulfonates, such as xylenesulfonates, methanesulfonates, propanesulfonates, naphthalene-1-sulfonates and naphthalene-2-sulfonates.

If an inventive basic compound is isolated as a salt, the corresponding free base form of that compound may be prepared by any suitable method known to the art, including treatment of the salt with an inorganic or organic base, suitably an inorganic or organic base having a higher pKa than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

Because the compounds of this invention may contain both acid and base moieties, pharmaceutically acceptable salts may be prepared by treating these compounds with an alkaline reagent or an acid reagent, respectively. Accordingly, this invention also provides for the conversion of one pharmaceutically acceptable salt of a compound of this invention, e.g., a hydrochloride salt, into another pharmaceutically acceptable salt of a compound of this invention, e.g., a sodium salt or a disodium salt.

Carboxylate functional groups of compounds of the present invention have coordinated mono or di-valent cations, where such cations may include, but are not limited to alkali metals, which may include, but are not limited to lithium (Li), sodium (Na), potassium, or mixtures thereof and the like.

Quarternary amine functional groups of compounds of the present invention, which are positively charged species, also may have coordinated anions, where such anions may include, but are not limited to halogens, which may include, but are not limited to chlorides, fluorides, bromides, iodides and the like.

Compounds of Formulas (I) to (XIII) of the present invention, also may form a zwitterion(s) (formerly called a dipolar ion), which is a neutral molecule with a positive and a negative electrical charge (i.e., not dipoles) at different locations within that molecule. Zwitterions are sometimes also called inner salts.

For solvates of the compounds of the invention, or salts thereof, that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The invention also includes various deuterated forms of the compounds of Formulas (I) to (XIII), respectively, or a pharmaceutically acceptable salt thereof. Each available hydrogen atom attached to a carbon atom may be independently replaced with a deuterium atom. A person of ordinary skill in the art will know how to synthesize deuterated forms of the compounds of Formulas (I) to (XIII), respectively, or a pharmaceutically acceptable salt thereof of the present invention. For example, deuterated materials, such as alkyl groups may be prepared by conventional techniques (see for example: methyl-d3-amine available from Aldrich Chemical Co., Milwaukee, Wis., Cat. No. 489,689-2).

The subject invention also includes isotopically-labeled compounds which are identical to those recited in Formulas (I) to (XIII), respectively, or a pharmaceutically acceptable salt thereof but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Because the compounds of the present invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“FimH inhibitor” or “FimH antagonist”, is used herein to refer to a compound that exhibits an HAI (hemagglutination inhibition) titer or EC_{>90} with respect to FimH function/activity of no more than about 100 μM and more typically not more than about 50 jtM, as measured in the FimH hemagglutination HAI assay described generally herein. “HAI or EC_{>90}” is that concentration of the FimH inhibitor/antagonist which reduces the bacterial agglutination of guinea pig red blood cells by greater than 90%. Certain compounds disclosed herein have been discovered to exhibit inhibition of this FimH function/activity. In certain embodiments, compounds will exhibit an EC_{>90} with respect to FimH of no more than about 10 NM; in further embodiments, compounds will exhibit an EC_{>90} with respect to FimH of no more than about 1 μM; in yet further embodiments, compounds will exhibit an EC_{>90} with respect to FimH of not more than about 1 NM; in yet further embodiments, compounds will exhibit an EC_{>90} with respect to FimH of not more than about 250 nM; in yet further embodiments, compounds will exhibit an EC_{>90} with respect to FimH of not more than about 100 nM in yet further embodiments, compounds will exhibit an EC_{>90} with respect to FimH of not more than about 50 nM in yet further embodiments, compounds will exhibit an EC_{>90} with respect to FimH of not more than about 10 nM, as measured in the FimH assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. Examples of prodrugs suitable for compounds disclosed herein are optionally substituted acetyl, amide, and phosphate groups, wherein said groups are attached to one or more of the hydroxyl groups on the molecule.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier (s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal inhalation, intranasal, and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

As used herein, the term “compound(s) of the invention” means a compound of Formulas (I) to (XIII), respectively (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

The present invention relates to a compound of Formulas (I) to (XIII), which definition referred herein includes, but are not limited to the following related sub-generic Formulas (II) and (XIII).

The alternative definitions for the various groups and substitutent groups of Formulas (I) to (XIII), respectively, or a pharmaceutically acceptable salt thereof, provided throughout the specification are intended to particularly describe each compound species disclosed herein, individually, as well as groups of one or more compound species. The scope of this invention includes any combination of these group and substituent group definitions.

The alternative definitions for the various groups and substitutent groups of Formulas (I) to (XIII), respectively, or a pharmaceutically acceptable salt thereof, provided throughout the specification are intended to particularly describe each compound species disclosed herein, individually, as well as groups of one or more compound species. The scope of this invention includes any combination of these group and substituent group definitions.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the rectum, lung, vaginal cavity, ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for urinary tract infection involving administration of one of the compounds described herein, increased therapeutic benefit may result by also providing the patient with another therapeutic agent for urinary tract infection. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating FimH-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of FimH-mediated disorders.

Specific diseases to be treated by the compounds, compositions, and methods disclosed herein include bacterial infections, Crohn's Disease, and irritable bowel syndrome (IBS). In certain embodiments, the bacterial infection is a urinary tract infection.

Besides being useful for human treatment, certain compounds and formulations disclosed herein may also be useful for veterinary treatment of companion animals, exotic animals and farm animals, including mammals, and the like. More preferred animals include horses, dogs, and cats.

It is noted that each compound herein can be properly named in multiple ways. For example, 4′-((R)-hydroxy((2R,3S,4S,5S,6R)-3,4,5-trihydroxy-6-(hydroxymethyl)tetrahydro-2H-pyran-2-yl)methyl)-3′-methylbiphenyl-3-carbonitrile and 4′-[(α-D-mannopyranosyl)-(R)-hydroxymethyl]-3′-methyl-[1,1′biphenyl]-3-carbonitrile are two ways to describe Example 51. These names are equivalent and can be used interchangeably to correctly describe the identical structure.

### List of Abbreviations

Ac=acetyl; Ac₂O=acetic anhydride; Bn=benzyl; BnBr=benzyl bromide; OsO₄=osmium tetraoxide; BCl₃=boron trichloride; NaIO₄=sodium periodate; CuSO₄=copper sulfate; n-BuiLi=n-butyl lithium; Cy=cyclohexyl; dba=dibenzylideneacetone; DCI=4,5-dicyanoimidazole; DDTT=3-((dimethylaminomethylidene)amino)-3H-1,2,4-dithiazole-5-thione; DMA=N,N-dimethylacetamide; DMAP=4-Dimethylaminopyridine; DMOCP=2-chloro-5,5-dimethyl-2-oxo-1,3,2-dioxaphosphorinane; DMP=Dess-Martin periodinane; DMTr=dimethoxytrityl=(4-methoxyphenyl)₂ (phenyl)methyl; Piv=pivaloyl ═(CH₃)₃C—C(═O)—; NaOH=sodium hydroxide; NaH=sodium hydride; M=molar; nM=nanomolar; μM=micromolar mL=milliliter; h=hour; min.=minute; HCl=hydrogen chloride; H₂O=water; MS=mass spectrometry; LCMS=Liquid chromatography/mass spectrometry; ES+=electrospray positive ionization; ¹H-NMR=proton nuclear magnetic resonance; ¹³C-NMR=carbon-13 nuclear magnetic resonance; ³¹P-NMR=phosphorous-31 nuclear magnetic resonance; MHz=megahertz; H=hydrogen; RT=rt=room temperature; ° C.=Celsius; Br₂=bromine; NaHSO₃=sodium bisulfite; NMP=N-Methyl-2-pyrrolidone; NMM=N-methyl morpholine; NMO=N-methyl morpholine N-oxide; MW=microwave; KF=potassium fluoride; Pd (dppf)Cl₂=[1,1′-bis (diphenylphosphino)ferrocene]palladium (II) dichloride; PE=petroleum ether; EtOAc=EA=EtOAc; CDCl₃=deuterated chloroform; DMSO-d₆=dimethyl sulfoxide deuterated-6; CD₃CN=deuterated acetonitrile; LTBA=lithium tri (tert-butoxy)aluminium hydride=LiAlH (Ot-Bu)₃; MeOH=methanol; NaOMe=sodium methoxide; D₂O=deuterated water; prep-HPLC=preparative high pressure liquid chromatography, also known as preparative high performance liquid chromatography; DMSO=dimethyl sulfoxide; MeCN═CH₃CN=acetonitrile; CH₃I=methyl iodide; NH₃=ammonia; NH₄OH=ammonium hydroxide; NIS═N-iodosuccinimide; DMF=N, N-dimethylformamide; K₃PO₄=potassium phosphate, tribasic; N₂=nitrogen; Py=pyridine; THF=tetrahydrofuran; Cs₂CO₃=cesium carbonate; Na₂CO₃=sodium carbonate; NaHCO₃=sodium bicarbonate; Na₂SO₄=sodium sulfate; TEA=triethylamine; TBSCl=tert-butyldimethylsilyl chloride; TMSCl=trimethylsilyl chloride; TMS=trimethylsilyl; TMSOTf=trimethylsilyl triflate; TFA=trifluoroacetic acid; DCM=CH₂Cl₂=dichloromethane; Hunig's base=DIPEA=iPr₂NEt=N, N-diisopropylethylamine; K₂CO₃=potassium carbonate; KOAc=potassium acetate; μl=microliter; g=gram; mg=milligram.

### General Synthetic Methods for Preparing Compounds

The following general schemes shown below are used for synthesis of the compounds described in the Examples. Four methods utilize a Pd-mediated ‘Suzuki’ cross-coupling between an appropriately functionalized aryl or heteroaryl mannoside (bromide, boronic acid or boronate ester), and an arene or heteroarene with appropriate complementary functionality (bromide, boronic acid or boronate ester). Schemes A and B employ a mannoside substituted with an aryl bromide, and an aryl boronate ester. Schemes C and D employ a mannoside substituted with an aryl boronate ester, and an aryl bromide. Other Examples detailed within this application are obtained by the method shown in Scheme E, via a copper-mediated triazole forming ‘Click’ chemistry reaction between an appropriately functionalized aryl or heteroaryl mannoside azide and an alkyne.

### General Procedure for Suzuki Coupling/Deprotection Sequence

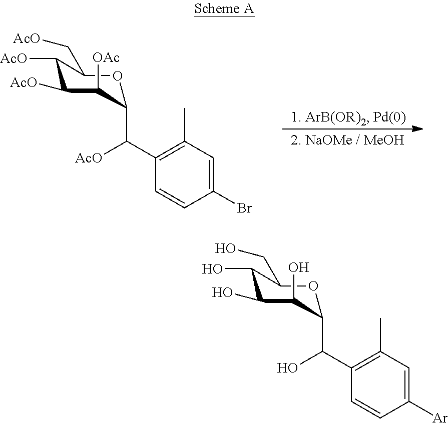

Scheme A uses acetate esters as protecting groups on the mannoside. After Suzuki coupling, the ester groups are removed via methanolysis.

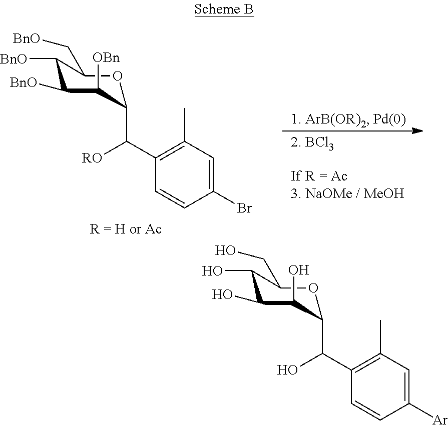

Scheme B uses benzyl ethers as protecting groups on the mannoside. After Suzuki coupling, the benzyl groups are removed with BCl₃. If R═Ac, the remaining acetate group is removed via methanolysis.

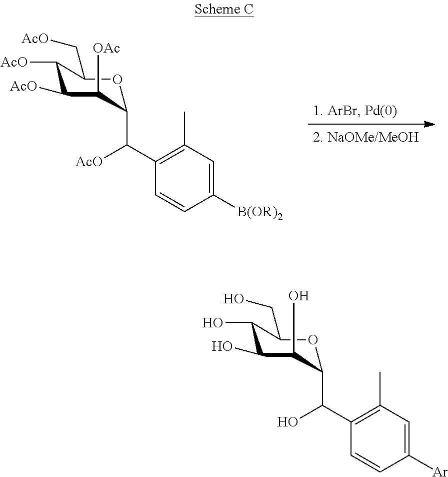

Scheme C uses acetate esters as protecting groups on the mannoside. After Suzuki coupling, the ester groups are removed via methanolysis.

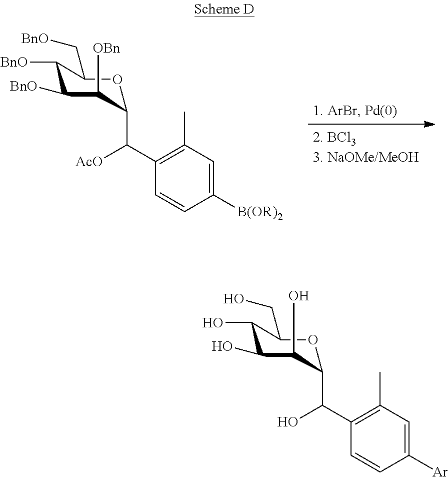

Scheme D uses benzyl ethers as protecting groups on the mannoside. After Suzuki coupling, the benzyl groups are removed with BCl₃. The remaining acetate group is removed via methanolysis.

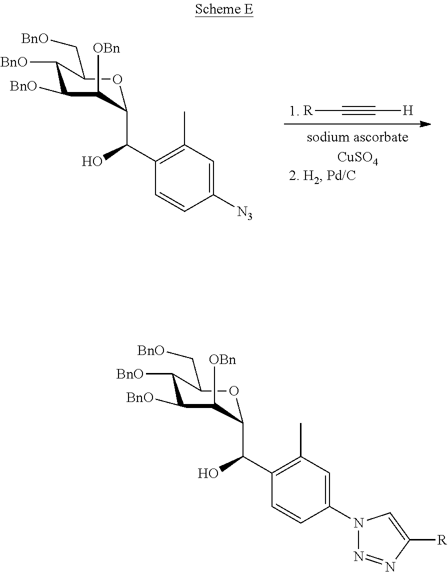

Scheme E uses benzyl ethers as protecting groups on the mannoside. After Click reaction, the benzyl groups are removed with catalytic hydrogenation. The remaining acetate group is removed via methanolysis.

General Procedure for the Suzuki Coupling Reactions

To a solution of the mannoside aryl bromide or boronate (1.0 equiv) in dioxane/water (V/V=5/1) were added aryl boronic acid (boronate) or aryl halide (1.1 equiv), Cs₂CO₃ (3 equiv) and Pd (PPh₃)₄ (0.05 equiv) at rt. The resulting mixture was degassed three times. The flask was then placed in an oil bath preheated to 80° C., and allowed to stir for the time specified (typically 30 min to 2 h). The reaction mixture was then cooled to rt and solvents were evaporated under reduced pressure. The crude residue was then purified by silica gel chromatography. The product was then deprotected by either protocol A or B.

Deprotection Protocol A

Acetate protecting groups were removed by dissolving the partially purified mannoside from the Suzuki reaction into MeOH (3-5 mL), and cooling to 0° C. [1M] Sodium methoxide in MeOH was added dropwise until a pH of 9-10 was achieved. After 5 min, the ice bath was removed and the reaction mixture was stirred for the time specified. Upon completion, the reaction was quenched with water (4 drops) and concentrated under reduced pressure. The crude product was purified by Prep-HPLC with different conditions.

Deprotection Protocol B

Benzyl ethers were deprotected by adding BCl₃ (8.0 eqv, 1M in CH₂Cl₂) to a solution of the partially purified mannoside from the Suzuki reaction in dichloromethane (10 mL). The reaction was stirred for the time specified at −78° C. After completion, the reaction was quenched by methanol (1 mL) at −78° C. Then the reaction was warmed to rt and concentrated under reduced pressure to afford the de-benzyl compound. If there is a benzylic acetate present, the acetyl group was then removed by the method described in protocol A. If no acetate, the crude product was purified by Prep-HPLC with different conditions.

Deprotection Protocol C

Alternatively, benzyl ethers were deprotected by adding 10% wt. Pd/C (0.5 eqv) to a solution of the partially purified mannoside from the Suzuki reaction into MeOH (3-5 mL). The reaction was stirred under 1 atm of H₂ for the time specified. Upon deprotection, the reaction was filtered and the filtrate was concentrated in vacuo. If present, the benzylic acetyl group was then removed by the method described in protocol A. If no acetate, the crude product was purified by Prep-HPLC with different conditions.

Deprotection Protocol D

The benzylic hydroxy group was protected by dissolving the compound (0.10 mmol) in pyridine (2 mL) and cooling to 0° C. Next, Ac₂O (1.5 eqv per hydroxyl) and DMAP (0.05 eqv) is added, and the reaction is stirred. After 15 min, the reaction is brought to rt and stirred for the time specified. Upon completion the reaction is cooled to 0° C., and quenched with MeOH (1 mL). The pyridine is removed in vacuo, and the residue is then redissolved in CH₂Cl₂, (5 mL) and washed successively with water (5 mL), 1 N aq. HCl (2×5 mL), water (5 mL), saturated aq. NaHCO₃ (5 mL×2), and brine (5 mL), dried over Na₂SO₄, and concentrated in vacuo. Purification by column chromatography on silica gel (EtOAc-hexanes gradient) yields the acetate protected intermediate. Deprotection protocol B is then followed to remove benzyl ethers followed by protocol A to remove the benzylic acetate.

Deprotection Protocol E

Alternatively, acetate protecting groups were removed by dissolving the partially purified mannoside into MeOH (3-5 mL), and K₂CO₃ (0.25 equivalents) was added, and the reaction was stirred at rt for the time specified. Upon completion, the reaction was neutralized with H⁺ exchange resin (DOWEX 50WX4-100). The resin was filtered, and the filtrate was concentrated in vacuo. The crude product was purified by Prep-HPLC with different conditions.

### C-Mannoside Building Block Synthesis

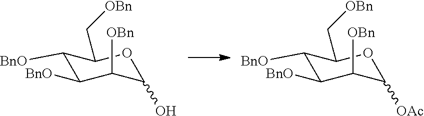 [SMILES: OC1OC(COCc2ccccc2)C(OCc2ccccc2)C(OCc2ccccc2)C1OCc1ccccc1 ; CC(=O)OC1OC(COCc2ccccc2)C(OCc2ccccc2)C(OCc2ccccc2)C1OCc1ccccc1]

### Acetyl 2,3,4,6-tetra-O-benzyl-α-D-mannopyranoside

Commercially available 2,3,4,6-tetra-O-benzyl-α-D-mannopyranoside (8.9 g, 16.46 mmol) and DMAP (101 mg, 0.823 mmol) were dissolved into dry pyridine (50 mL). The reaction was cooled to 0° C., and Ac₂O (2.33 mL, 24.69 mmol) was added dropwise. After 15 min, the reaction was brought to rt, and stirred for 16 h. Upon completion, the reaction was cooled to 0° C., and quenched with MeOH (1 mL). The pyridine was removed in vacuo, and the residue was then redissolved in CH₂Cl₂, (50 mL) and washed successively with water (20 mL), 1 N aq. HCl (2×20 mL), water (20 mL), saturated aq. NaHCO₃ (20 mL×2), brine (20 mL), dried over Na₂SO₄, and concentrated in vacuo. Purification by column chromatography on silica gel (EtOAc-hexanes gradient) gave the desired compound in 95% yield.

¹H NMR (300 MHz, CDCl₃) δ ppm 7.15-7.40 (m, 21H), 6.88-6.91 (m, 1H), 6.81 (t, J=2.4 Hz, 1H), 6.73-6.76 (m, 1H), 5.58 (d, J=1.8 Hz, 1H), 4.90 (d, J=10.8 Hz, 1H), 4.78 (s, 2H), 4.64-4.69 (m, 3H), 4.52 (d, J=10.8 Hz, 1H), 4.45 (d, J=12.3 Hz, 1H), 4.05-4.18 (m, 2H), 3.94 (t, J=2.4 Hz, 1H), 3.77-3.85 (m, 2H), 3.64-3.70 (m, 1H), 2.27 (s, 3H). MS (ESI): found: [M+Na], 697.2.

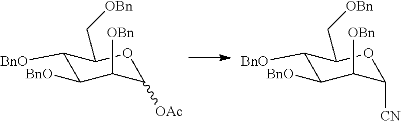 [SMILES: CC(=O)OC1OC(COCc2ccccc2)C(OCc2ccccc2)C(OCc2ccccc2)C1OCc1ccccc1 ; N#CC1OC(COCc2ccccc2)C(OCc2ccccc2)C(OCc2ccccc2)C1OCc1ccccc1]

### 2,3,4,6-Tetra-O-benzyl-α-D-mannopyranosyl cyanide

The compound from the previous step (9.45 g, 16.22 mmol) was dissolved into dry CH₃CN (175 mL) under N₂, and the reaction was cooled to 0° C. Trimethylsilyl cyanide (6.11 mL, 0.049 mmol) was added, followed by the dropwise addition of BF₃—OEt₂ (0.41 mL, 3.24 mmol). After 30 min, solvents were evaporated, and the resulting residue was redissolved in CH₂Cl₂ (50 mL) and washed with H₂O (30 mL), 1M aq. HCl (30 mL) and brine (30 mL). The organic fractions were combined and, dried over Na₂SO₄ and concentrated in vacuo. A mix of α- and β-anomers were obtained, and were easily separated by column chromatography on silica gel (EtOAc-hexanes gradient) gave the desired α-mannoside in 51% yield (and the β-mannoside byproduct in 27% yield).

MS (ESI): found [M+Na+], 572.2.

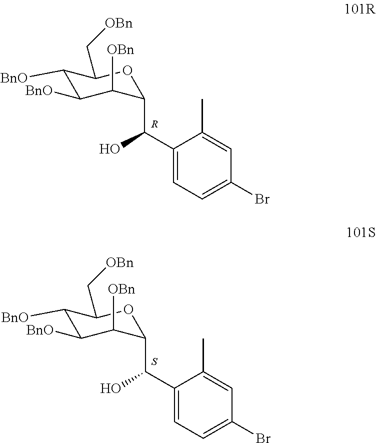

### (R)-(4-bromo-2-methylphenyl)((2R,3S,4S,5R,6R)-3,4,5-tris(benzyloxy)-6-((benzyloxy)methyl)tetrahydro-2H-pyran-2-yl)methanol (Intermediates 101R) and (S)-(4-bromo-2-methylphenyl)((2R,3S,4S,5R,6R)-3,4,5-tris(benzyloxy)-6-((benzyloxy)methyl)tetrahydro-2H-pyran-2-yl)methanol (101S)

Synthesis A: At −78° C., DIBAL/Hexane (1.0 M, 11.3 mL) was added dropwise into the solution of 2,3,4,6-tetra-O-benzyl-α-D-mannopyranosyl cyanide (4.97 g, 9.04 mmol) in CH₂Cl₂ (150 mL) under N₂. The mixture was stirred for 30 min, maintaining a temperature of −78° C. Then, the reaction was diluted with CH₂Cl₂ (150 mL) then acidified with the addition of 0.2 N aq. HCl (400 mL). The reaction was stirred 10 min at rt, and then filtered through CELITE® (to help break up emulsion) into a separatory funnel. The distinct layers were separated, and the aqueous layer was then extracted an additional time with CH₂Cl₂. The two organic fractions were combined and washed 2× with H₂O (100 mL). The organic layers were dried over Na₂SO₄, which also cleared up any remaining emulsion, and then concentrated to give intermediate carbaldehyde as the crude product. Due to its instability, this intermediate was used without further purification after drying 30 min to 1 h under high vacuum.

Concurrent to the synthesis of the carbaldehyde, into another flask containing 4-bromo-2-methyl-iodobenzene (9.04 mL, 63.29 mmol) in anhydrous Et₂O (150 mL) under N₂, was added dropwise n-BuLi/Hexanes (2.5 M, 21.7 mL) at −78° C. After 1 h, the crude carbaldehyde (in 25 mL anhydrous Et₂O) was quickly added via cannula. The mixture was stirred at −78° C. for 30 min, and was then slowly warmed to 0° C. over 1.5 h. Saturated aqueous NH₄Cl was used to quench the reaction, and the reaction was extracted with EtOAc (2×100 mL). The organic fractions were then combined and washed with brine (100 mL), dried over Na₂SO₄ and concentrated in vacuo. The resulting residue was mixture of diastereoisomers, which were purified and separated by silica gel chromatography (EtOAc-hexane gradient elution), to give Intermediate 101R as a syrup in 16% yield (1.05 g, 1.45 mmol), and Intermediate 101S as a syrup in 20% yield (1.30 g, 1.80 mmol).

Formula: C₄₂H₄₃BrO₆ Exact Mass: 722.22 Molecular Weight: 723.69.

Analytical data for 101R: ¹H NMR (400 MHz, CDCl₃) δ ppm 7.28-7.41 (2m, 21H) 7.13-7.18 (m, 2H) 5.08 (d, J=5.1 Hz, 1H) 4.71 (2d, J=11.7 Hz, 1H) 4.56-4.64 (m, 3H) 4.49 (s, 2H) 4.40 (s, 2H) 4.21-4.28 (m, 1H) 4.13-4.18 (m, 1H) 4.10 (t, J=5.1 Hz, 1H) 3.94-3.99 (m, 1H) 3.89 (t, J=5.9 Hz, 1H) 3.70-3.83 (m, 2H) 3.49 (br. s., 1H) 2.29 (s, 3H); ESI-MS [M+Na]⁺ calcd for C₄₂H₄₃BrO₆Na⁺745.21, found 745.5 (100%), 747.5 (97.3%).

Analytical data for 101S: ¹H NMR (400 MHz, CDCl₃) δ ppm 7.16-7.37 (m, 23H) 5.06 (d, J=5.5 Hz, 1H) 4.67-4.73 (m, 1H) 4.44-4.62 (m, 8H) 4.03-4.11 (2m, 2H) 3.76-3.85 (m, 3H) 3.67-3.73 (m, 2H) 3.19 (br. s., 1H) 2.18 (s, 3H); ESI-MS [M+Na]⁺ calcd for C₄₂H₄₃BrO₆Na⁺745.21, found 745.5 (100%), 747.5 (97.3%).

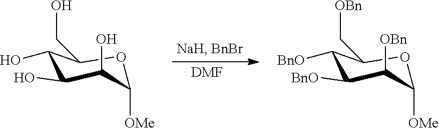

### Methyl 2,3,4,6-tetra-O-benzyl-α-D-mannopyranoside or (2R,3R,4S,5S,6S)-3,4,5-tris(benzyloxy)-2-((benzyloxy)methyl)-6-methoxytetrahydro-2H-pyran

To a stirred solution of commercially available methyl α-D-mannopyranoside (30.0 g, 0.155 mol) in dry DMF (1000 mL) cooled with an ice-water bath was added portionwise NaH (37.1 g, 0.928 mol, 60% in mineral oil). After addition, the reaction mixture was stirred at this temperature until the evolution of gas subsided (typically within 30 min). Benzyl bromide (158.7 g, 0.928 mol) was added portionwise to the reaction mixture over 30 min. After addition, the reaction mixture was stirred at this temperature for 2 h and then at rt overnight, when TLC analysis indicated that the reaction completed. The reaction mixture was carefully poured into ice water (2500 mL) while stirring, and the resulted mixture was extracted with dichloromethane (2500 mL×3). The combined organic layers were washed with brine, dried over anhydrous Na₂SO₄ and evaporated on a rotary evaporator to afford an oily residue, which was purified by column chromatography, eluting with EtOAc in PE (0-20%) to afford the pure title compound (71.0 g, 83% yield) as yellow oil. ESI-MS [M+Na]⁺ calcd for (C₃₅H₃₈O₆Na⁺) 577.27, found 577.0.

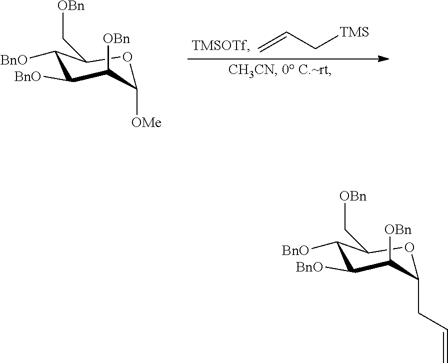

### (2R,3R,4R,5R,6R)-2-allyl-3,4,5-tris(benzyloxy)-6-((benzyloxy)methyl)tetrahydro-2H-pyran

To a stirred solution of Methyl 2,3,4,6-tetra-O-benzyl-α-D-mannopyranoside (78.0 g, 0.141 mol) in dry CH₃CN (100 mL) cooled with an ice-water bath was added dropwise allyltrimethylsilane (33.0 g, 0.288 mol) and trimethylsilyl trifluoromethanesulfonate (16.0 g, 0.07 mol). After addition, the reaction mixture was stirred at rt overnight. After completion, the reaction mixture was carefully poured into ice water (200 mL) while stirring, and the resulting mixture was extracted with EtOAc (300 mL×3). The combined organic layers were washed with brine (200 mL), dried over anhydrous Na₂SO₄, filtered. The filtrate was evaporated on a rotary evaporator to afford an oily residue, which was purified by column chromatography, eluting with EtOAc in PE (10:1) to give the pure title compound (68.0 g, 84% yield) as colorless oil. ESI-MS [M+Na]⁺ calcd for (C₃₇H₄₀O₅Na⁺⁾ 587.29, found 587.30.

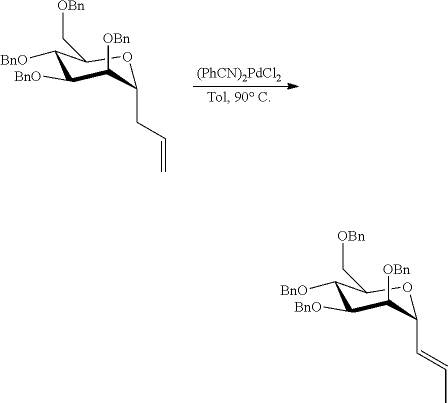

### (2R,3R,4R,5R,6R)-3,4,5-tris(benzyloxy)-2-(benzyloxymethyl)-6-((E)-prop-1-enyl)tetrahydro-2H-pyran

To a solution of (2R,3R,4R,5R,6R)-2-allyl-3,4,5-tris(benzyloxy)-6-((benzyloxy)methyl)tetrahydro-2H-pyran (68.0 g, 0.12 mol) dissolved in dried toluene (350 mL) was added Pd (PhCN)₂Cl₂ (7.0 g, 0.018 mmol) under N₂ atmosphere. The resulting mixture was heated at 90° C. overnight under N₂ atmosphere. After completion, the reaction was cooled to rt and concentrated under reduced pressure. The residue was purified by column chromatography, eluting with EtOAc in PE (16:1) to give the title compound (48.0 g, 71% yield) as yellow oil. MS (ESI+) calcd for (C₃₇H₄₀O₅Na⁺⁾[M+Na]⁺587.29, found 587.30.

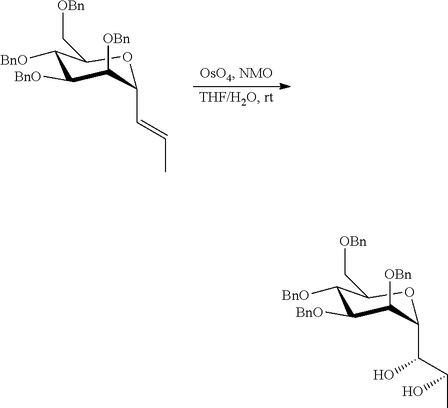

### (1S,2R)-1-((2R,3S,4S,5R,6R)-3,4,5-tris(benzyloxy)-6-(benzyloxymethyl)tetrahydro-2H-pyran-2-yl)propane-1,2-diol

To a solution of (2R,3R,4R,5R,6R)-3,4,5-tris(benzyloxy)-2-(benzyloxymethyl)-6-((E)-prop-1-enyl)tetrahydro-2H-pyran (48 g, 0.085 mol) and 4-methylmorpholine N-oxide (40 g, 0.157 mol) in mixed system of THF/water (100 mL/100 mL) was added OsO₄ (5 g, in 70 mL t-BuOH) at rt. The resulting mixture was stirred overnight at rt. The reaction mixture was poured into saturated Na₂S₂O₃ solution (300 mL) and extracted with EtOAc (300 mL×3), dried over Na₂SO₄ and filtered. The filtrate was concentrated under vacuum to get a residue which was purified by column chromatography, eluting with EtOAc in dichloromethane (ratio from 1/10 to 1/5) to give the title compound (34.0 g, 68% yield) as a white solid. ¹H NMR MS (ESI+) calcd for (C₃₇H₄₂O₇Na⁺⁾ [M+Na]⁺621.29, found 621.30.

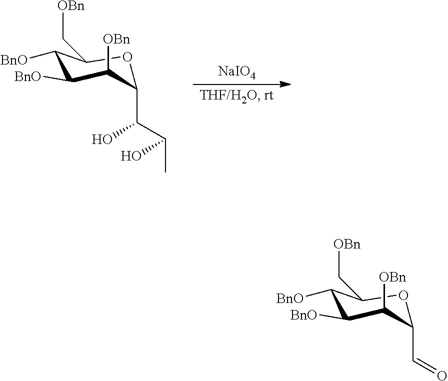

### 2,3,4,6-Tetra-O-benzyl-α-D-mannopyranosyl carbaldehyde or (2S,3S,4S,5R,6R)-3,4,5-tris(benzyloxy)-6-((benzyloxy)methyl)tetrahydro-2H-pyran-2-carbaldehyde

To the solution of (1S,2R)-1-((2R,3S,4S,5R,6R)-3,4,5-tris(benzyloxy)-6-(benzyloxymethyl)tetrahydro-2H-pyran-2-yl)propane-1,2-diol (13.0 g, 21.74 mmol) in THF/H₂O (120 mL/120 mL) was added NaIO₄ (13.0 g, 60.75 mmol) and the reaction mixture was stirred under N₂ for 3 h at rt. Upon completion, the reaction was quenched with ice water (100 mL) and extracted with CH₂Cl₂ (250 mL×3). The combined organic layers were washed with brine (100 mL), dried over anhydrous Na₂SO₄ and filtered. The filtrate was concentrated under reduced pressure to give the title compound which was used directly for the next steps without further purification. ESI-MS [M+Na]⁺ calcd for (C₃₅H₃₆NaO₆ Na⁺) 575.24, found 575.20.

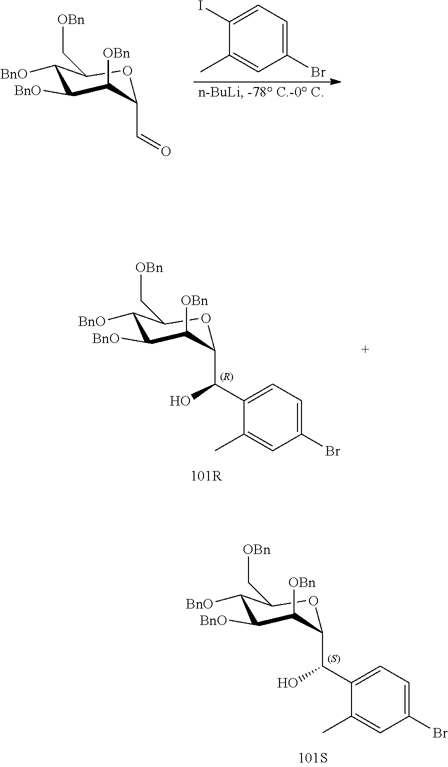

### (R)-(4-bromo-2-methylphenyl)((2R,3S,4S,5R,6R)-3,4,5-tris(benzyloxy)-6-((benzyloxy)methyl)tetrahydro-2H-pyran-2-yl)methanol (101R) and (S)-(4-bromo-2-methylphenyl)((2R,3S,4S,5R,6R)-3,4,5-tris(benzyloxy)-6-((benzyloxy)methyl)tetrahydro-2H-pyran-2-yl)methanol (101S)

Synthesis B: Into a flask containing 4-bromo-1-iodo-2-methylbenzene (22.6 g, 76.1 mmol) in anhydrous Et₂O (200 mL) under N₂, was added n-BuLi/Hexanes (2.5 M, 26 mL, 65.23 mmol) dropwise at −78° C. After 1 h, the freshly prepared crude 2,3,4,6-Tetra-O-benzyl-α-D-mannopyranosyl carbaldehyde (12.0 g, 21.74 mmol) dissolved in Et₂O (90 mL) was added via cannula over a period of 5 minutes. The mixture was stirred at −78° C. for 30 min, and then slowly warmed to 0° C. over a period of 1.5 h. The reaction mixture was quenched with saturated aq. NH₄Cl and extracted with EtOAc (250 mL×3). The combined organic phase was washed with brine (100 mL), dried over Na₂SO₄ and filtered. The filtrate was concentrated under reduced pressure and the residue was purified by silica gel chromatography (phase A: PE, phase B: CH₂Cl₂/EtOAc/PE (20/1/2)) to give the 101R (4.0 g, 26% yield for two steps) as light yellow oil and 101S (8.0 g, 51% yield for two steps) as light yellow oil.

Formula: C₄₂H₄₃BrO₆ Exact Mass: 722.22 Molecular Weight: 723.69

Analytical data for Intermediate 101R: ¹H NMR (300 MHz, CDCl₃) δ 7.41-7.28 (m, 21H), 7.18-7.13 (m, 2H), 5.08 (d, J=5.1 Hz, 1H), 4.71 (2d, J=11.7 Hz, 1H), 4.64-4.56 (m, 3H), 4.49 (s, 2H), 4.40 (s, 2H), 4.28-4.21 (2m, 1H), 4.18-4.13 (m, 1H), 4.10 (t, J=5.1 Hz, 1H), 3.99-3.94 (m, 1H), 3.89 (t, J=5.9 Hz, 1H), 3.83-3.70 (m, 2H), 3.49 (br. s., 1H), 2.29 (s, 3H). ESI-MS [M+Na+] calcd for (C₄₂H₄₃BrO₆Na) found: 745.5 (100%), 747.5 (97.3%).

Formula: C₄₂H₄₃BrO₆ Exact Mass: 722.22 Molecular Weight: 723.69

Analytical data for Intermediate 101 S: ¹H NMR (300 MHz, CDCl₃) δ 7.37-7.16 (m, 23H), 5.06 (d, J=5.5 Hz, 1H), 4.73-4.67 (m, 1H), 4.62-4.44 (m, 7H), 4.11-4.03 (m, 2H), 3.85-3.76 (m, 3H), 3.73-3.67 (m, 2H), 3.19 (br. s., 1H), 2.18 (s, 3H). ESI-MS [M+Na]⁺ calcd for C₄₂H₄₃BrO₆Na⁺745.21, found 745.5 (100%), 747.5 (97.3%).

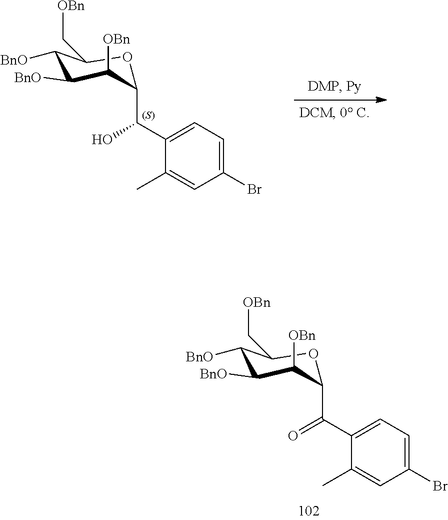

### (4-bromo-2-methylphenyl)((2S,3S,4S,5R,6R)-3,4,5-tris(benzyloxy)-6-((benzyloxy)methyl)tetrahydro-2H-pyran-2-yl)methanone (Intermediate 102)

To a stirred solution of Intermediate 101 S in dry CH₂Cl₂ (200 mL) was added dried pyridine (0.79 g, 0.01 mol) under N₂ at 0° C. Dess-Martin periodinane (3.4 g, 0.08 mol) was added portionwise, and the reaction mixture was kept at 0° C. for 1 hour, and then allowed to warm to 15° C. over an additional 1.5 hours. The reaction flask was cooled in an ice bath, and a 1:1 mixture of 10% solution of Na₂S₂O₃ (30 mL) and saturated solution of NaHCO₃ (30 mL) was added, and the reaction was stirred for 5 min at rt. The layers were then separated and the aqueous layer was extracted with CH₂Cl₂ (20 mL×3). The organic fractions were combined and washed with the solution of NaHCO₃ dried over Na₂SO₄, and concentrated in vacuo without heating to afford the desired ketone (2.03 g) as crude yellow oil which was directly used to next step without further purification.

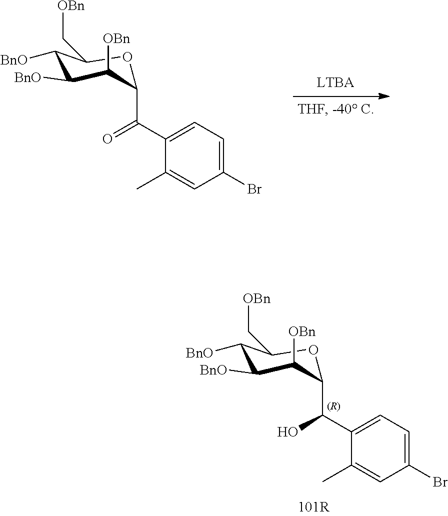

### (R)-(4-bromo-2-methylphenyl)((2R,3S,4S,5R,6R)-3,4,5-tris(benzyloxy)-6-((benzyloxy)methyl)tetrahydro-2H-pyran-2-yl)methanol (Intermediate 101R)

To a stirred solution of Intermediate 102 (2.03 g, 2.8 mmol) in dried THF (200 mL) was added LTBA (8.2 mL, 8.45 mmol) under N₂ at −40° C. The mixture was warmed to 0° C. and stirred an additional 1 h. When TLC analysis indicated that the reaction completed, the reaction mixture was diluted with EtOAc (400 mL). Saturated solution of potassium sodium tartrate (200 mL) was added, and the mixture was vigorously stirred for 1 h at rt. The organic layers were separated with NaHCO₃ (150 mL×3), dried over Na₂SO₄, filtered and the filtrate was concentrated under vacuum. The residue was purified by column chromatography (phase A: PE, phase B: CH₂Cl₂/EtOAc/PE (20/1/2)) to afford Intermediate 101R (1.62 g, 80% yield) as yellow oil.

Analytical data—as reported above.

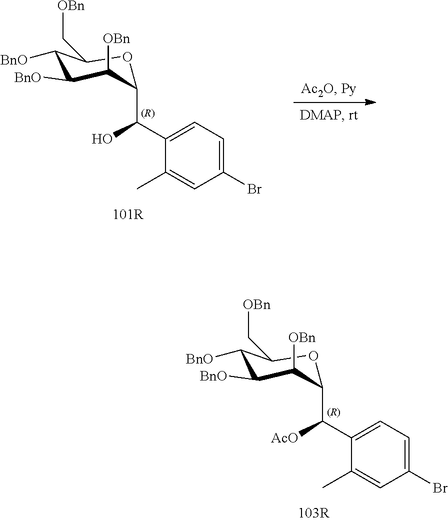

### (R)-(4-bromo-2-methylphenyl)((2R,3S,4S,5R,6R)-3,4,5-tris(benzyloxy)-6-((benzyloxy)methyl)tetrahydro-2H-pyran-2-yl)methyl acetate (Intermediate 103R

Dimethylaminopyridine (21 mg, 0.17 mmol) and Intermediate 101R (2.45 g, 3.39 mmol) were dissolved in dry pyridine (10 mL) under N₂, and the reaction was cooled to 0° C. Acetic anhydride (518 mg, 5.08 mmol) was added dropwise within 5 min. After stirring for 1 h at rt, the reaction mixture was cooled to 0° C. and quenched with MeOH (2 mL), and pyridine was evaporated in vacuo. The residue was re-dissolved in CH₂Cl₂ (30 mL) and washed successively with water (30 mL), 1 N aq. HCl (30 mL×2), water (30 mL), then dried over Na₂SO₄ and filtered. The filtrate was concentrated under reduced pressure and the residue was purified by silica gel chromatography, eluting with EtOAc-PE (0-20%) to afford the title compound (2.5 g, 97% yield) as yellow oil.

### (S)-(4-bromo-2-methylphenyl)((2R,3S,4S,5R,6R)-3,4,5-tris(benzyloxy)-6-((benzyloxy)methyl)tetrahydro-2H-pyran-2-yl)methyl acetate

(Intermediate 103S) was obtained from Intermediate 101 S via an analogous procedure.

Formula: C₄₄H₄₅Br₁NaO₇ Exact Mass; 764.23, Molecular Weight: 765.73.

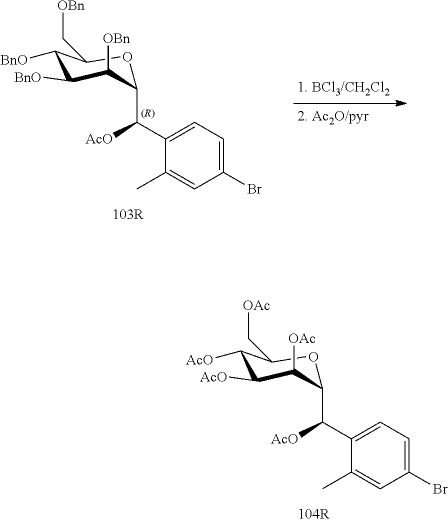

### (2R,3R,4S,5R,6R)-2-((R)-acetoxy(4-bromo-2-methylphenyl)methyl)-6-(acetoxymethyl)tetrahydro-2H-pyran-3,4,5-triyl triacetate (Intermediate 104R)

Intermediate 103R was dissolved in dry CH₂Cl₂ (15 mL) under N₂, and the reaction mixture was cooled to −78° C. Boron trichloride (356 mL, 1M in CH₂Cl₂, 3.56 mmol) was added dropwise, and the reaction mixture was stirred for 30 min. Upon completion, the reaction was quenched by the addition of methanol (2 mL). The reaction mixture was concentrated in vacuo and the residue (˜150 mg, contained de-Ac compound) was re-dissolved in dry pyridine (3 mL) under N₂, and the reaction was cooled to 0° C. Dimethylaminopyridine (3 mg, 0.019 mmol) was added, followed by acetic anhydride (230 mg, 2.3 mmol), and the reaction mixture was stirred for 5 min at 0° C. and then brought to rt After 1 h, the reaction was cooled again to 0° C., and quenched with MeOH (2 mL). Pyridine was removed in vacuo, and the residue was then re-dissolved in CH₂Cl₂ (25 mL) and washed successively with water (10 mL), 1 N aq. HCl (10 mL×2), water (10 mL), dried over Na₂SO₄ and filtered. The filtrate was concentrated in vacuo and the residue was purified by silica gel chromatography, eluting with EtOAc in PE to afford the desired the title compound (200 mg, 94% yield) as a white solid.

Formula: C₂₄H₂₉BrNaO₁₁ Exact Mass; 572.09, Molecular Weight: 573.38.

Analytical data for Intermediate 104R: ¹H NMR (300 MHz, CDCl₃) δ ppm 7.36-7.32 (m, 2H), 7.24-7.21 (2m, 1H), 6.19 (d, J=6.9 Hz, 1H), 5.54 (t, J=3.3 Hz, 1H), 5.37 (dd, J₁=9.0 Hz, J₂=3.6 Hz, 1H), 5.18 (t, J=8.5 Hz, 1H), 4.26-4.21 (2m, 2H), 4.02-3.91 (2m, 2H), 2.43 (s, 3H), 2.14 (s, 3H), 2.08 (s, 6H), 2.03 (s, 3H), 1.97 (s, 3H). ESI-MS [M+Na]⁺ calcd for (C₂₄H₂₉BrNaO₁₁Na⁺), 595.08, found 595.2 (100%), 597.3 (97.3%).

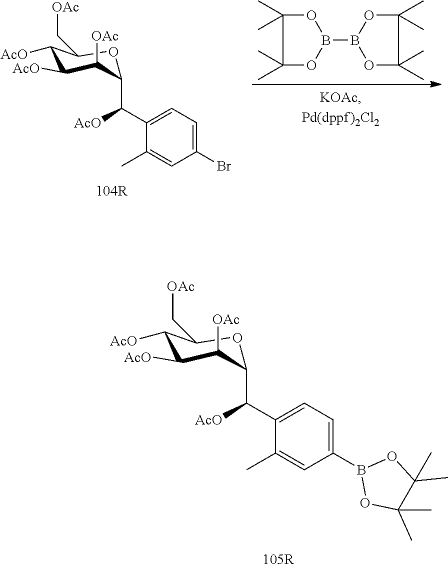

### (2R,3R,4S,5R,6R)-2-((R)-acetoxy(2-methyl-4-(4,4,5,5-tetramethyl-1,3,2-dioxaborolan-2-yl)phenyl)methyl)-6-(acetoxymethyl)tetrahydro-2H-pyran-3,4,5-triyl triacetate (Intermediate 105R)

Under N₂ atmosphere, a mixture of Intermediate 104R (500 mg, 0.87 mmol), bis(pinacolato)diboron (243 mg, 0.96 mmol), KOAc (256.1 mg, 2.61 mmol) and Pd (dppf)Cl₂ (71 mg, 0.09 mmol) in dioxane (10 mL) was heated at 90° C. with stirring for 3 h. Upon completion, the reaction was cooled to rt and concentrated under reduced pressure. The residue was purified by silica gel chromatography, eluting with EtOAc in PE (0˜30%) to afford the title compound (480 mg, 89% yield) as light yellow oil.

ESI-MS [M+H]⁺ calcd for (C₃₀H₄₁BO₁₃H) 621.26, found 621.0.

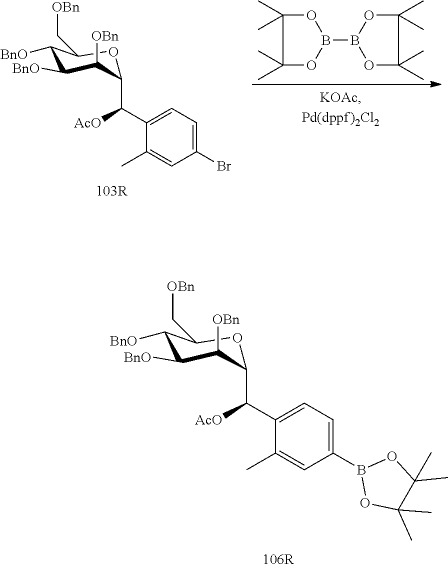

### (R)-(2-methyl-4-(4,4,5,5-tetramethyl-1,3,2-dioxaborolan-2-yl)phenyl)((2R,3S,4S,5R,6R)-3,4,5-tris(benzyloxy)-6-((benzyloxy)methyl)tetrahydro-2H-pyran-2-yl)methyl acetate (Intermediate 106R)

Under N₂ atmosphere, the mixture of Intermediate 103R (1.2 g, 1.57 mmol), bis(pinacolato)diboron (438 mg, 1.72 mmol), KOAc (462 mg, 4.71 mmol) and Pd (dppf)Cl₂ (131 mg, 0.16 mmol) in dioxane (10 mL) was stirred for 8 h at 80° C. Upon completion, the reaction was cooled to rt. The reaction mixture was concentrated under reduced pressure. The residue was purified by silica gel chromatography, eluting with EtOAc in PE (0-17%) to afford the desired boronate (920 mg, 72% yield) as yellow oil.

ESI-MS [M+NH₄]⁺ calcd for (C₅₀H₅₇BO₉NH₄⁺) 830.41, found 830.5.

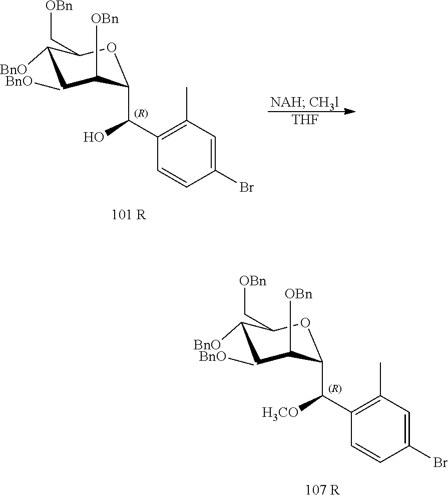

### (2R,3R,4S,5S,6R)-3,4,5-tris(benzyloxy)-2-((benzyloxy)methyl)-6-((R)-(4-bromo-2-methylphenyl)(methoxy)methyl)tetrahydro-2H-pyran (Intermediate 107R)

Intermediate 101R (65 mg, 0.090 mmol) was dissolved in dry THF (2 mL) under N₂. Mel (11.2 μL, 0.179 mmol) was added, and the reaction was cooled to 0° C. NaH (60% dispersion in mineral oil; 5.4 mg, 0.134 mmol) was added portionwise, and the reaction was stirred for 1 h at 0° C. Upon completion, the reaction was diluted with EtOAc (5 mL), and quenched by the addition of ice water (5 mL), and subsequently extracted with EtOAc (3×5 mL). The organic fractions were combined, dried over Na₂SO₄, and concentrated in vacuo. The residue was purified by column chromatography on silica gel (EtOAc-hexane gradient elution) to afford Intermediate 107R in 68% yield.

Formula: C₄₃H₄₅BrO₆ Exact Mass: 736.24 Molecular Weight: 737.72.

Analytical data for Intermediate 107R: ¹H NMR (400 MHz, CDCl₃) δ ppm 7.08-7.26 (m, 21H) 6.96-7.00 (m, 2H) 6.70 (d, J=8.2 Hz, 1H) 4.81 (2d, J=10.6 Hz, 1H) 4.55 (d, J=12.0 Hz, 1H) 4.36-4.53 (m, 6H) 4.31 (2d, J=12.0 Hz, 1H) 3.93-4.02 (m, 2H) 3.83-3.90 (m, 2H) 3.75 (br. s., 1H) 3.63 (d, J=4.3 Hz, 1H) 2.98 (s, 3H) 2.15 (s, 3H); ESI-MS [M+Na]⁺ calcd for C₄₃H₄₅BrO₆Na⁺759.23, found 759.5 (100%), 761.5 (97.3%).

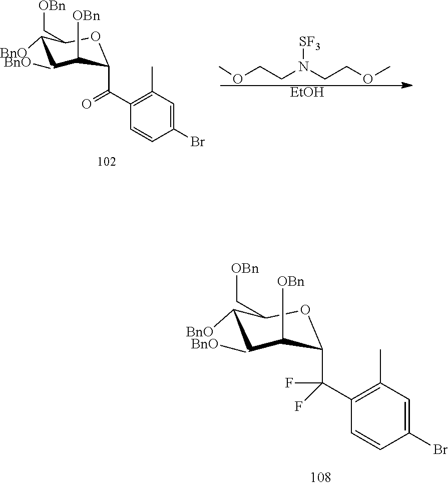

### (2R,3R,4S,5S,6S)-3,4,5-tris(benzyloxy)-2-((benzyloxy)methyl)-6-((4-bromo-2-methylphenyl)difluoromethyl)tetrahydro-2H-pyran (Intermediate 108)

Following a reported literature protocol (Link, J. O.; J. Med. Chem. 2014, 57 (5), 2033-2046), to a flask containing Intermediate 102 (86.0 mg, 0.119 mmol) under N₂, was added neat DEOXO-FLUOR® [bis (2-methoxyethyl)aminosulfur trifluoride] (1.2 mL, 0.96 mmol). One drop of EtOH was then added, and the reaction was warmed to 80° C. and allowed to stir for 1 to 2 days. The reaction was cooled to rt, and quenched by the addition of ice water (0.25 mL), and subsequently neutralized with saturated aq. NaHCO₃. The reaction was further diluted with H₂O (10 mL), and the reaction mixture was extracted with EtOAc (3×5 mL). The organic fractions were combined, dried over Na₂SO₄, and concentrated in vacuo. The residue was purified by column chromatography on silica gel (EtOAc-hexane gradient elution) to afford Intermediate 108 in 54% yield.

Formula: C₄₂H₄₁BrF₂O₅ Exact Mass: 742.21 Molecular Weight: 743.67.

Analytical data for Intermediate 108: ¹H NMR (400 MHz, CDCl₃) δ ppm 7.19-7.28 (m, 18H) 7.13-7.17 (m, 4H) 7.06 (d, J=8.6 Hz, 1H) 4.72 (d, J=11.0 Hz, 1H) 4.57 (d, J=2.0 Hz, 2H) 4.52 (d, J=3.9 Hz, 2H) 4.45 (t, J=11.9 Hz, 2H) 4.34 (d, J=12.0 Hz, 1H) 3.96 (t, J=2.9 Hz, 1H) 3.82-3.94 (m, 3H) 3.52-3.67 (m, 3H) 2.25 (s, 3H); ESI-MS [M+Na]⁺ calcd for C₄₂H₄₁BrF₂O₅Na⁺765.20, found 765.4 (100%), 767.5 (97.3%).

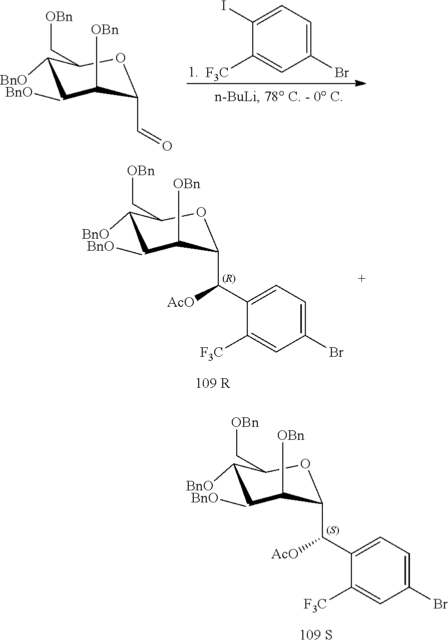

### (R)-(4-bromo-2-(trifluoromethyl)phenyl)((2R,3S,4S,5R,6R)-3,4,5-tris(benzyloxy)-6-(benzyloxymethyl)tetrahydro-2H-pyran-2-yl)methyl acetate (Intermediate 109R) and (S)-(4-bromo-2-(trifluoromethyl)phenyl)((2R,3S,4S,5R,6R)-3,4,5-tris(benzyloxy)-6-(benzyloxymethyl)tetrahydro-2H-pyran-2-yl)methyl acetate (Intermediate 109S)

Following the same procedure as described above for 101R and 101S, 2,3,4,6-tetra-O-benzyl-α-D-mannopyranosyl carbaldehyde (3.3 g, 5.3 mmol) was reacted with 4-bromo-1-iodo-2-(trifluoromethyl)benzene, followed by combiflash chromatography purification (Phase A: PE; phase B: CH₂Cl₂/EtOAc/PE=20:1:2, flow rate: 80 mL/min; gradient 30% B-70% B in 60 min. R-alcohol eluted at 30 min and S-alcohol eluted at 50 min) afforded R-alcohol (1.2 g, assumed, 26% for two steps) as light yellow oil and S-alcohol (1.2 g, assumed, 26% for two steps) as light yellow oil. Following the same procedure as described above, the alcohols were reacted with Ac₂O to afford the corresponding title compounds 109R and 109S (99% yield) as light yellow oil.

Formula: C₄₄H₄₂BrF₃O₇ Exact Mass: 818.21 Molecular Weight: 819.7.

Analytical data for Intermediate 109R: ¹H NMR (300 MHz, DMSO-d₆) δ 7.86 (d, J=1.8 Hz, 1H), 7.77-7.66 (m, 2H), 7.33-7.17 (m, 20H), 6.20 (d, J=6.3 Hz, 1H), 4.65 (d, J=11.4 Hz, 1H), 4.54-4.49 (m, 4H), 4.43-4.37 (m, 1H), 4.33-4.25 (m, 3H), 4.03-4.00 (m, 1H), 3.89-3.86 (m, 1H), 3.77-3.72 (m, 2H), 3.61-3.45 (m, 2H), 1.92 (s, 3H). ESI-MS [M+Na]⁺ calcd for (C₄₄H₄₂BrF₃O₇Na⁺) 841.20, found 841.40, 843.40.

Analytical data for Intermediate 109S: ¹H NMR (300 MHz, DMSO-d₆) δ 7.87 (d, J=1.8 Hz, 1H), 7.74-7.62 (m, 2H), 7.36-7.20 (m, 20H), 6.28 (d, J=6.0 Hz, 1H), 4.60-4.56 (m, 4H), 4.52 (s, 1H), 4.39 (d, J=12.0 Hz, 1H), 4.22-4.18 (m, 2H), 4.11-3.99 (m, 3H), 3.85-3.82 (m, 1H), 3.69-3.66 (m, 1H), 3.58-3.52 (m, 1H), 3.42-3.37 (m, 1H), 1.96 (s, 3H). ESI-MS [M+Na]⁺ calcd for (C₄₄H₄₂BrF₃O₇Na⁺) [M+Na]⁺841.20, found 841.0.

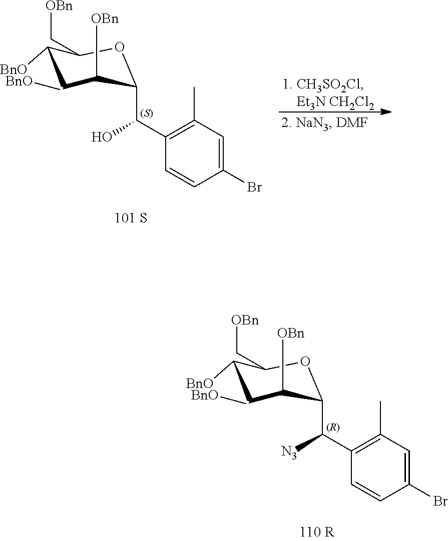

### (2R,3R,4R,5R,6R)-2-((R)-azido(4-bromo-2-methylphenyl)methyl)-3,4,5-tris(benzyloxy)-6-((benzyloxy)methyl)tetrahydro-2H-pyran (Intermediate 110R)

Following a modified procedure (J. Org. Chem. 2014, 79, 5636-5643), Intermediate 101 S (75.0 mg, 0.104 mmol) was dissolved into dry CH₂Cl₂ (1 mL) under N₂, and Et₃N (0.058 mL, 0.415 mmol) was added and the reaction was cooled to 0° C. Methanesulfonyl chloride (23.8 mg, 0.208 mmol) was diluted into dry CH₂Cl₂ (0.5 mL), and added dropwise over 10 min. After 30 min, the reaction mixture was poured into ice water (1 mL), then diluted with EtOAc (10 mL) and sequentially washed with chilled solutions of 1M aq. HCl (5 mL), H₂O (5 mL), saturated aq. NaHCO₃, and brine (5 mL). The reaction was dried over Na₂SO₄ and concentrated in vacuo at rt. After drying for 1 h, the residue was redissolved into dry DMF (1 mL), crushed NaN₃ (68.0 mg, 1.04 mmol) was added, and the reaction was heated to 60° C. for 16 h. The reaction was then cooled to rt, diluted with H₂O (10 mL) and extracted with 1:1 EtOAc:Et₂O (3×3 mL). The organic fractions were combined, dried over Na₂SO₄, and concentrated in vacuo. The residue was purified by column chromatography on silica gel (EtOAc-hexane gradient elution) to afford Intermediate 11 OR in 90% yield (with a minor impurity of elimination product).

Formula: C₄₂H₄₂BrN₃O₅ Exact Mass: 747.23 Molecular Weight: 748.70.

Analytical data for Intermediate 11 OR: ¹H NMR (400 MHz, CDCl₃) δ ppm 7.33 (d, J=8.6 Hz, 1H) 7.16-7.28 (m, 18H) 7.11-7.15 (m, 2H) 7.04 (dd, J=6.3, 2.7 Hz, 2H) 4.71 (2d, J=5.9 Hz, 1H) 4.35-4.57 (m, 6H) 4.13-4.31 (2m, 3H) 3.91-3.98 (m, 1H) 3.75-3.83 (m, 2H) 3.68-3.74 (m, 2H) 3.59 (dd, J=10.6, 4.3 Hz, 1H) 2.17 (s, 3H); ESI-MS [M+Na]⁺ calcd for C₄₂H₄₂BrN₃O₅Na⁺770.22, found 770.5 (100%), 772.5 (97.3%).

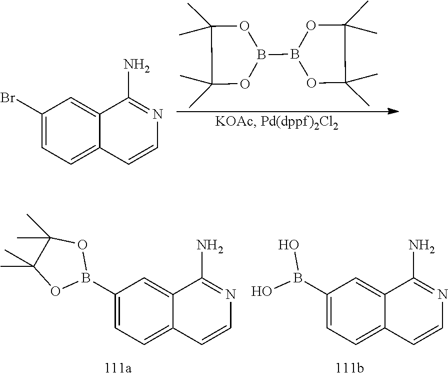

### 7-(4,4,5,5-tetramethyl-1,3,2-dioxaborolan-2-yl)isoquinolin-1-amine (Intermediate 111a) and isoquinolin-1-amine-7 boronic acid (Intermediate 111b)

KOAc (264 mg, 2.69 mmol) was activated by adding it to a round bottom flask, which was then heated to 250° C. under vacuum for 2 min, and then allowed to cool to rt under vacuum for an additional 10 min, after which time a N₂ atmosphere was continuously maintained. Dry DMSO (2 mL) was added, followed by the addition of commercially available 7-bromoisoquinolin-1-amine (150 mg, 0.67 mmol) and bis(pinacolato)diboron (256 mg, 1.0 mmol). Pd (dppf)Cl₂ (49.2 mg, 0.067 mmol) was added, and the reaction flask was evacuated under high vacuum and then repressurized with N₂ three times. The flask was then placed in an oil bath preheated to 80° C., and allowed to stir for 2.5 h. The reaction was cooled to rt, and solvents were evaporated under reduced pressure. The crude reaction residue was then redissolved into CH₂Cl₂, and allowed to sit for 5 min to allow for byproducts to precipitate. The precipitate was filtered off. Evaporation of the CH₂Cl₂ in vacuo resulted in more byproduct precipitation, and so the residue was redissolved in CH₂Cl₂ and the process was repeated until no further precipitation was observed. The crude brown residue was then diluted with H₂O (1 mL), and lyophilized to removed trace DMSO, resulting in a brown solid, which was comprised of a mix of Intermediate 111a and Intermediate 111b, as determined by LCMS. This crude mixture was used without further purification.

Intermediate 111a Formula: C₁₅H₁₉BN₂O₂ Exact Mass: 270.15 Molecular Weight: 270.13.

Analytical data for Intermediate 111a: ESI-MS [M+H]⁺ calcd for C₁₅H₁₉BN₂O₂H⁺271.16, found 271.3.

Intermediate 111b Formula: C₉H₉BN₂O₂ Exact Mass: 188.08 Molecular Weight: 187.99.

Analytical data for Intermediate 111b: ESI-MS [M+H]⁺ calcd for C₉H₉BN₂O₂H⁺189.08, found 189.2.

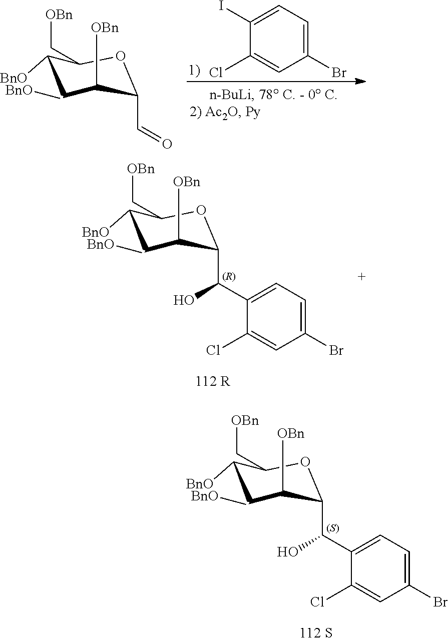

### (R)-(4-bromo-2-chlorophenyl)((2R,3S,4S,5R,6R)-3,4,5-tris(benzyloxy)-6-((benzyloxy)methyl)tetrahydro-2H-pyran-2-yl)methanol (Intermediate 112R) and (S)-(4-bromo-2-chlorophenyl)((2R,3S,4S,5R,6R)-3,4,5-tris(benzyloxy)-6-((benzyloxy)methyl)tetrahydro-2H-pyran-2-yl)methanol (Intermediate 112 S)

The condensation between the lithiated arene and carboxaldehyde as shown above followed the standard procedure previously disclosed for the synthesis of 101R/S. The mixture of diastereomeric alcohols was purified using column chromatography on silica gel, first using an EtOAc-hexanes gradient elution to separate and collect the top isomer, and then re-chromatographing the collected bottom isomer (EtOAc-DCM gradient elution) to further remove impurities. The impure top isomer was next acetylated, following the protocol disclosed for the synthesis of 103R, and purified by column chromatography on silica gel (EtOAc-hexanes gradient elution) to to give intermediate 112R in 3% yield.

Formula: C₄₃H₄₂BrClO₇ Exact Mass: 784.18 Molecular Weight: 784.16 ¹H NMR (400 MHz, chloroform-d₃) δ ppm 7.40 (d, J=2.0 Hz, 1H), 7.15-7.26 (m, 19H), 7.09-7.13 (m, 2H), 7.05 (d, J=8.6 Hz, 1H), 6.24 (d, J=7.0 Hz, 1H), 4.66 (d, J=11.3 Hz, 1H), 4.41-4.56 (m, 6H), 4.29-4.34 (m, 1H), 4.26 (dd, J=7.0, 3.9 Hz, 1H), 3.80-3.89 (m, 2H), 3.71-3.78 (m, 1H), 3.60-3.68 (m, 2H), 3.50 (dd, J=10.8, 2.9 Hz, 1H), 1.83 (s, 3H); ESI-MS [M+Na]⁺ calcd for C₄₃H₄₂BrClO₇Na⁺807.17, 809.17 found 807.4, 809.2.

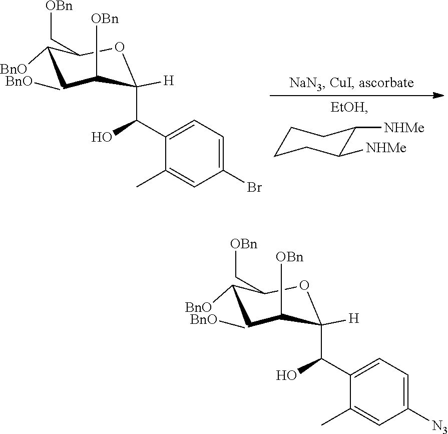

### (2R,3S,4R,5R,6R)-2-((R)-(4-azido-2-methylphenyl)(hydroxy)methyl)-4,5-bis(benzyloxy)-6-((benzyloxy)methyl)tetrahydro-2H-pyran-3-ol (Intermediate 113R)

Similar to a reported literature protocol (Synlett 2005, (No. 14), 2209), compound 101R (192 mg, 0.27 mmol) was dissolved in EtOH (8 mL) and H₂O (1 mL) under N₂. Trans-N,N′-dimethyl-1,2-cyclohexanediamine (11.4 mg, 0.080 mmol) was added, followed by NaN₃ (26.1 mg, 0.40 mmol), CuI (10.2 mg, 0.054 mmol), and sodium L-ascorbate (5.3 mg, 0.027 mmol). The reaction was refluxed for 30 min (monitoring by LCMS, as the starting material and product have the same R_{f} on TLC in all systems tried), then upon completion was cooled to rt, diluted with EtOAc: hexanes (5 mL), and quenched by the addition of saturated aq. NH₄Cl (3 mL). The biphasic mixture was stirred 1 h at rt. The solution was then filtered through a pad of CELITE®, and was subsequently washed with EtOAc (20 mL). The filtrate was transferred to a separatory funnel, the phases were separated, and the aqueous phase was extracted with EtOAc: hexanes (3×10 mL). The organic fractions were then combined and washed with saturated aq. NaHCO₃, (15 mL) and brine (2×15 mL), dried over Na₂SO₄, and concentrated in vacuo, to give intermediate 113R in 71% yield.

Formula: C₄₂H₄₃N₃O₆ Exact Mass: 685.32 Molecular Weight: 685.82

¹H NMR (400 MHz, chloroform-d₃) δ ppm 7.26-7.37 (m, 19H), 7.17 (d, J=2.3 Hz, 2H), 6.73-6.80 (m, 2H), 5.08 (d, J=5.1 Hz, 1H), 4.65-4.69 (m, 1H), 4.55-4.61 (m, 3H), 4.34-4.46 (m, 4H), 4.19 (d, J=4.7 Hz, 1H), 4.10 (d, J=3.5 Hz, 2H), 3.98 (d, J=2.7 Hz, 1H), 3.83-3.88 (m, 1H), 3.73-3.80 (m, 1H), 3.64-3.72 (m, 1H), 2.29 (s, 3H); ESI-MS [M+Na]⁺ calcd for C₄₂H₄₃N₃O₆Na⁺708.30 found 708.5.

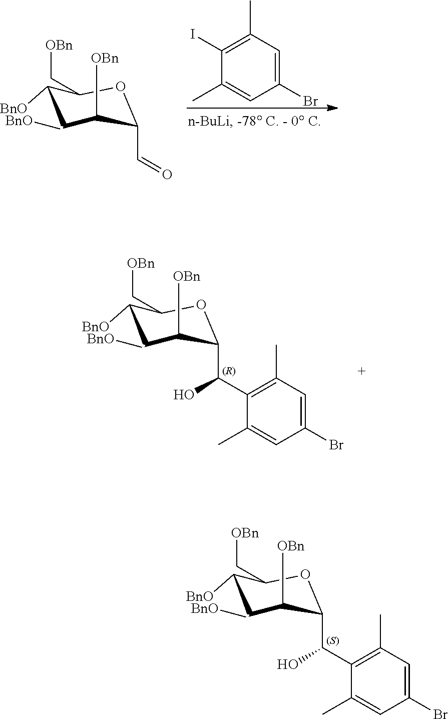

### (R)-(4-bromo-2,6-dimethylphenyl)((2R,3S,4S,5R,6R)-3,4,5-tris(benzyloxy)-6-((benzyloxy)methyl)tetrahydro-2H-pyran-2-yl)methanol and (S)-(4-bromo-2,6-dimethylphenyl)((2R,3S,4S,5R,6R)-3,4,5-tris(benzyloxy)-6-((benzyloxy)methyl)tetrahydro-2H-pyran-2-yl)methanol (Intermediate 114R/S)

Following the same procedure as described above for the synthesis of 101R and 101S, 2,3,4,6-tetra-O-benzyl-α-D-mannopyranosyl carbaldehyde (1.0 g, 1.6 mmol) was reacted with 5-bromo-2-iodo-1,3-dimethylbenzene, and then the crude product was purified by combiflash chromatography (Phase A: PE; phase B: CH₂Cl₂/EtOAc/PE=20:1:2, flow rate: 80 mL/min; gradient 70% B-100% B in 60 min. R isomer came out at 30 min and S-isomer came out at 44 min) afforded R-isomer (0.48 g, assumed, 36% for two steps) as light yellow oil and S-isomer (0.48 g, assumed, 36% for two steps) as light yellow oil.

Formula: C₄₃H₄₅BrO₆ Exact Mass: 736.24 Molecular Weight: 737.72.

Analytical data for R isomer: ¹H NMR (400 MHz, CDCl₃) δ 7.35-7.16 (m, 18H), 7.18-7.14 (m, 2H), 7.10-7.08 (m, 2H), 5.14 (d, J=8.4 Hz, 1H), 4.61-4.49 (m, 6H), 4.40-4.36 (m, 1H), 4.31 (2s, 2H), 4.10 (dd, J=6.4 Hz, 2.8 Hz, 1H), 3.96-3.90 (m, 2H), 3.81-3.79 (m, 1H), 3.65 (d, J=6.0 Hz, 2H), 2.35 (s, 6H). ESI-MS [M+Na]⁺ calcd for (C₄₃H₄₅BrO₆Na⁺⁾ 759.24, found 759.20.

Analytical data for S isomer: ¹H NMR (400 MHz, CDCl₃) δ 7.34-7.20 (m, 18H), 7.08 (s, 2H), 7.00-6.95 (m, 2H), 5.15 (d, J=9.2 Hz, 1H), 4.76 (d, J=11.2 Hz, 1H), 4.58-4.40 (m, 6H), 4.36-4.30 (m, 2H), 4.15-4.09 (m, 1H), 3.95-3.83 (m, 2H), 3.79-3.73 (m, 2H), 3.49 (t, J=3.5 Hz, 1H), 2.28 (s, 6H). ESI-MS [M+Na]⁺ calcd for (C₄₃H₄₅BrO₆Na+) 759.24, found 759.20.

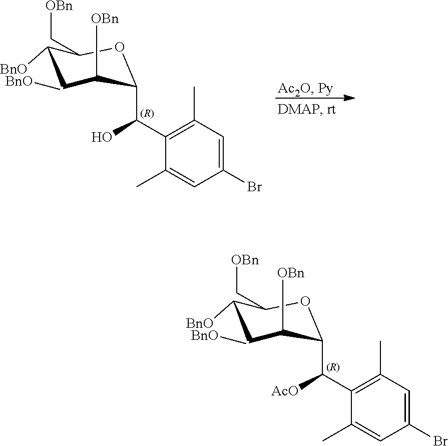

### (R)-(4-bromo-2,6-dimethylphenyl)((2R,3S,4S,5R,6R)-3,4,5-tris(benzyloxy)-6-((benzyloxy)methyl)tetrahydro-2H-pyran-2-yl)methyl acetate (Intermediate 115R)

Following the same procedure as described for the synthesis of Intermediate 104, reaction of Intermediate 114R with Ac₂O to afford the acetate in 80% yield as light yellow oil. ESI-MS [M+Na]⁺ calcd for (C₄₂H₄₃BrO₆Na⁺⁾ 801.25, found 801.25.

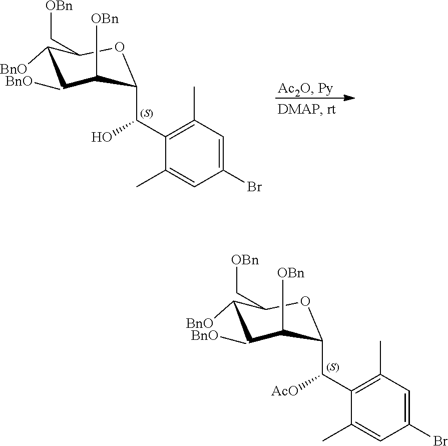

### (S)-(4-bromo-2,6-dimethylphenyl)((2R,3S,4S,5R,6R)-3,4,5-tris(benzyloxy)-6-((benzyloxy)methyl)tetrahydro-2H-pyran-2-yl)methyl acetate (Intermediate 115S)

Following the same procedure as described above, reaction of 101S with Ac₂O to afford the acetate in 98% yield as light yellow oil.

The invention is further illustrated by the following examples.

### EXAMPLE 1

### (2R,3S,4S,5S,6R)-2-((R)-hydroxy(4-(isoquinolin-5-yl)-2-methylphenyl)methyl)-6-(hydroxymethyl)tetrahydro-2H-pyran-3,4,5-triol

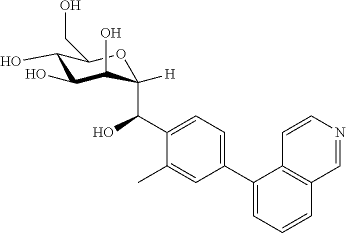

Following Scheme A, Intermediate 104R and commercially available 5-isoquinolinylboronic acid were reacted via the standard Suzuki coupling procedure (4 h at 80° C.), followed by deprotection protocol A (30 min at rt). The resulting residue was purified by HPLC (C18, 15*150 mm column; eluent: acetonitrile/water (0.05% TFA) to give 1 in 38% yield.

Formula: C₂₃H₂₅NO₆ Exact Mass: 411.17 Molecular Weight: 411.45

Analytical data: ¹H NMR (400 MHz, methanol-d₄) δ ppm 9.83 (s, 1H) 8.52 (d, J=7.4 Hz, 2H) 8.37 (d, J=6.7 Hz, 1H) 8.07-8.20 (m, 2H) 7.75 (d, J=8.2 Hz, 1H) 7.32-7.41 (m, 2H) 5.30 (d, J=7.0 Hz, 1H) 4.29 (t, J=2.9 Hz, 1H) 4.17 (dd, J=7.0, 2.3 Hz, 1H) 3.99-4.11 (m, 1H) 3.67-3.74 (m, 4H) 2.55 (s, 3H); ESI-MS [M+H]⁺ calcd for C₂₃H₂₅NO₆H⁺412.18 found 412.3.

### EXAMPLE 2

### 7-(4-((R)-hydroxy((2R,3S,4S,5S,6R)-3,4,5-trihydroxy-6-(hydroxymethyl)tetrahydro-2H-pyran-2-yl)methyl)-3-methylphenyl)isoquinolin-1 (2H)-one

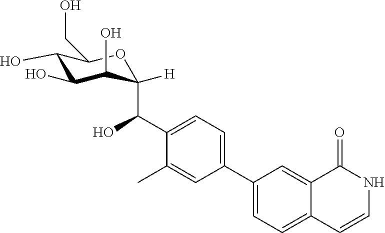

Following Scheme A, Intermediate 104R and commercially available 1-hydroxy-isoquinoline-7-boronate ester were reacted via the standard Suzuki coupling procedure (1.5 h at 80° C.), followed by deprotection protocol A (2 h at rt). The resulting residue was purified by HPLC (C18, 15*150 mm column; eluent: acetonitrile/water (0.05% TFA) to give 2 in 47% yield.

Formula: C₂₃H₂₅NO₇ Exact Mass: 427.16 Molecular Weight: 427.45

Analytical data: ¹H NMR (400 MHz, methanol-d₄) δ ppm 8.54 (s, 1H) 8.00 (d, J=8.6 Hz, 1H) 7.72 (d, J=8.2 Hz, 1H) 7.62-7.67 (m, 1H) 7.52-7.61 (m, 2H) 7.18 (d, J=7.0 Hz, 1H) 6.70 (d, J=7.0 Hz, 1H) 5.25 (d, J=6.7 Hz, 1H) 4.26 (br. s., 1H) 4.09-4.15 (m, 1H) 4.05 (br. s., 1H) 3.64-3.75 (m, 4H) 2.52 (s, 3H); ESI-MS [M+H]⁺ calcd for C₂₃H₂₅NO₇H⁺428.17 found 428.4, (410.3 M-18+H), (855.6 2M+H).

### EXAMPLE 3

### (2R,3S,4S,5S,6R)-2-((R)-(4-(1-aminoisoquinolin-7-yl)-2-methylphenyl)(hydroxy)methyl)-6-(hydroxymethyl)tetrahydro-2H-pyran-3,4,5-triol

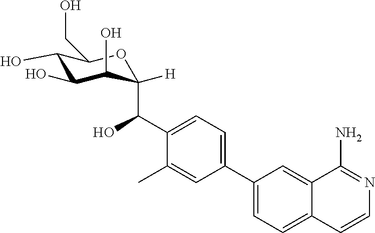

Following Scheme A, Intermediate 104R and isoquinolin-1-amine-7-boronate ester/acid (111a/b) were reacted via the standard Suzuki coupling procedure (1.5 h at 80° C.), followed by deprotection protocol A (30 min at rt). The resulting residue was purified by HPLC (C18, 15*150 mm column; eluent: acetonitrile/water (0.05% TFA) to give 3 in 26% yield.

Formula: C₂₃H₂₆N₂O₆ Exact Mass: 426.18 Molecular Weight: 426.46

Analytical data: ¹H NMR (400 MHz, methanol-d₄) δ ppm 8.71 (s, 1H) 8.26 (d, J=8.2 Hz, 1H) 7.97 (d, J=8.6 Hz, 1H) 7.62-7.72 (m, 3H) 7.54 (d, J=7.0 Hz, 1H) 7.24 (d, J=7.0 Hz, 1H) 5.27 (d, J=7.0 Hz, 1H) 4.27 (t, J=2.9 Hz, 1H) 4.13 (dd, J=6.8, 2.2 Hz, 1H) 4.01-4.07 (m, 1H) 3.63-3.73 (m, 4H) 2.55 (s, 3H); ESI-MS [M+H]⁺ calcd for C₂₃H₂₅NO₇H⁺427.19 found 427.4.

### EXAMPLE 4

### 7-(4-((R)-hydroxy((2R,3S,4S,5S,6R)-3,4,5-trihydroxy-6-(hydroxymethyl)tetrahydro-2H-pyran-2-yl)methyl)-3-methylphenyl)-3,4-dihydroisoquinolin-1 (2H)-one

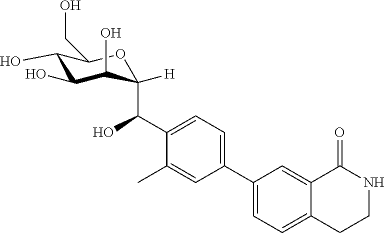

Following Scheme B, Intermediate 101R and commercially available 1-hydroxy-isoquinoline-7-boronate ester were reacted via the standard Suzuki coupling procedure (1.5 h at 80° C.), followed by deprotection protocol C (16 h at rt). The resulting residue was purified by HPLC (C18, 15*150 mm column; eluent: acetonitrile/water (0.05% TFA) to give 4 in 46% yield.

Formula: C₂₃H₂₇NO₇ Exact Mass: 429.18 Molecular Weight: 429.46

Analytical data: ¹H NMR (400 MHz, methanol-d₄) δ ppm 8.18 (d, J=1.6 Hz, 1H) 7.75 (dd, J=7.8, 2.0 Hz, 1H) 7.61 (d, J=7.8 Hz, 1H) 7.44-7.53 (m, 2H) 7.37 (d, J=7.8 Hz, 1H) 5.24 (d, J=7.0 Hz, 1H) 4.25 (t, J=2.5 Hz, 1H) 4.10 (dd, J=6.7, 2.0 Hz, 1H) 4.05 (br. s, 1H) 3.63-3.74 (m, 4H) 3.52 (t, J=6.7 Hz, 2H) 3.01 (t, J=6.7 Hz, 2H) 2.49 (s, 3H); ESI-MS [M+H]⁺ calcd for C₂₃H₂₇NO₇H⁺430.19 found 430.4, (412.4 M-18+H), (859.6 2M+H).

### EXAMPLE 5

### (2R,3S,4S,5S,6R)-2-((R)-hydroxy(2-methyl-4-(2-methyl-1,2,3,4-tetrahydroisoquinolin-5-yl)phenyl)methyl)-6-(hydroxymethyl)tetrahydro-2H-pyran-3,4,5-triol

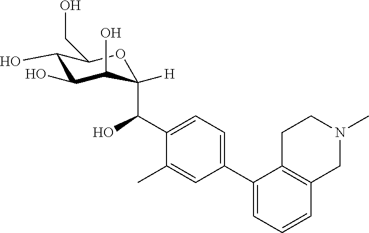

Following Scheme B, Intermediate 101R and commercially available 5-isoquinolinylboronic acid were reacted via the standard Suzuki coupling procedure (2.5 h at 80° C.), followed by deprotection protocol C (24 h at rt). The resulting residue was purified by HPLC (C18, 15*150 mm column; eluent: acetonitrile/water (0.05% TFA) to give 5 in 3% yield.

Formula: C₂₄H₃₁NO₆ Exact Mass: 429.22 Molecular Weight: 429.51

Analytical data: ¹H NMR (400 MHz, methanol-d₄) δ ppm 7.46 (d, J=8.2 Hz, 1H) 7.13 (t, J=8.0 Hz, 1H) 6.94-7.04 (m, 4H) 5.13 (d, J=7.0 Hz, 1H) 4.16 (t, J=2.7 Hz, 1H) 4.01 (dd, J=7.0, 2.3 Hz, 1H) 3.91-3.97 (m, 1H) 3.79 (s, 2H) 3.53-3.62 (m, 4H) 2.74 (dd, J=15.3, 4.3 Hz, 4H) 2.50 (s, 3H) 2.36 (s, 3H); ESI-MS [M+H]⁺ calcd for C₂₄H₃₁NO₆H⁻ 430.22 found 430.4.

### EXAMPLE 6

### 4′-((R)-amino((2R,3S,4R,5S,6R)-3,4,5-trihydroxy-6-(hydroxymethyl)tetrahydro-2H-pyran-2-yl)methyl)-N,3′-dimethylbiphenyl-3-carboxamide

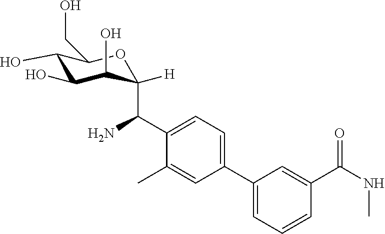

Following Scheme B, Intermediate 110R and commercially available N-methyl-3-(4,4,5,5-tetramethyl-[1,3,2]dioxaborolan-2-yl)-benzamide were reacted via the standard Suzuki coupling procedure (1.5 h at 80° C.). This was followed by deprotection protocol C (24 h at rt). However, under these conditions the benzyl ether protecting group remained intact; only reduction of the azide to the amine occurred. Thus, deprotection protocol B was subsequently employed (3 h at −78° C.). The resulting residue was purified by HPLC (C18, 15*150 mm column; eluent: acetonitrile/water (0.05% TFA) to give 110R in 3% yield.

Formula: C₂₂H₂₈N₂O₆ Exact Mass: 416.19 Molecular Weight: 416.47

Analytical data: ¹H NMR (400 MHz, methanol-d₄) δ ppm 8.09 (s, 1H) 7.91 (d, J=8.2 Hz, 1H) 7.77-7.82 (m, 2H) 7.50-7.61 (m, 3H) 4.98 (d, J=3.5 Hz, 1H) 4.30 (dd, J=10.0, 3.3 Hz, 1H) 4.14-4.22 (m, 1H) 3.96 (d, J=5.1 Hz, 1H) 3.77 (s, 2H) 3.67 (dd, J=11.9, 3.7 Hz, 1H) 3.41 (d, J=9.4 Hz, 1H) 2.95 (s, 3H) 2.59 (s, 3H); ESI-MS [M+H]⁺ calcd for C₂₂H₂₅N₂O₆H⁺417.20 found 417.4, (400.4 M-18+H), (833.7 2M+H).

### EXAMPLE 7

### 4′-((R)-methoxy((2S,3S,4S,5S,6R)-3,4,5-trihydroxy-6-(hydroxymethyl)tetrahydro-2H-pyran-2-yl)methyl)-N,3′-dimethylbiphenyl-3-carboxamide

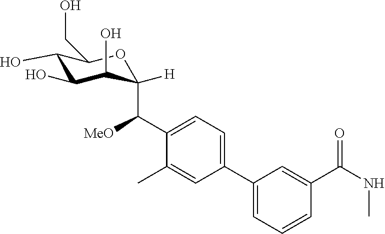

Following Scheme B, Intermediate 107R and commercially available N-methyl-3-(4,4,5,5-tetramethyl-[1,3,2]dioxaborolan-2-yl)-benzamide were reacted via the standard Suzuki coupling procedure (1.5 h at 80° C.), followed by deprotection protocol C (2 h at rt). The resulting residue was purified by HPLC (C18, 15*150 mm column; eluent: acetonitrile/water (0.05% TFA). to give 7 in 48% yield.

Formula: C₂₃H₂₉NO₇ Exact Mass: 431.19 Molecular Weight: 431.48

Analytical data: ¹H NMR (400 MHz, methanol-d₄) δ ppm 8.08 (s, 1H) 7.78 (d, J=7.8 Hz, 2H) 7.45-7.57 (m, 4H) 4.85 (d, J=5.5 Hz, 1H) 4.20 (br. s, 1H) 4.00-4.06 (m, 2H) 3.62-3.80 (m, 4H) 3.24 (s, 3H) 2.95 (s, 3H) 2.49 (s, 3H); ESI-MS [M+H]⁺ calcd for C₂₃H₂₉NO₇H⁻ 432.20 found 432.4, (400.4 M-32+H), (863.7 2M+H).

### EXAMPLE 8

### 4′-(methoxy((2S,3S,4S,5S,6R)-3,4,5-trihydroxy-6-(hydroxymethyl)tetrahydro-2H-pyran-2-yl)methyl)-N,2′-dimethylbiphenyl-3-carboxamide

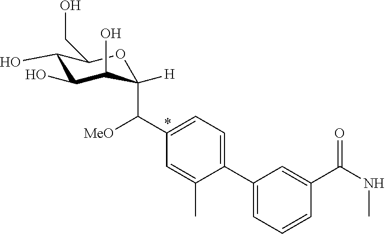

8 was synthesized as a byproduct of the synthesis of 7, wherein the meta-methyl regioisomer was traced back to an alternate procedure for synthesizing 101R/S. Investigating solvent effects, 101R/S was synthesized using the exact same protocol as detailed above, the only change being that anhydrous THF was used in place of Et₂O, v/v. This change gave rise to a large amount of byproduct, corresponding to (2′,3′,4′,6′-tetra-O-benzyl-α-D-mannopyranosyl)-(4″-iodo-3″-methylphenyl)-methan-1(R/S)-ol, which was not separable by column chromatography on silica gel. The mixture of the unwanted 4-iodo-3-methylphenyl mannoside byproduct and the desired 4-bromo-2methylphenyl mannoside (101R) was then carried forward through the methylation step, described in the synthesis of pure intermediate 107R. Thus, 107R and its 2-methyl regioisomer were coupled via the standard Suzuki coupling procedure with commercially available N-methyl-3-(4,4,5,5-tetramethyl-[1,3,2]dioxaborolan-2-yl)-benzamide (1.5 h at 80° C.), followed by deprotection protocol C (2 h at rt). Purification by HPLC (C18, 15*150 mm column; eluent: acetonitrile/water (0.05% TFA), was able to separate the regioisomers, and 8 was isolated 11% yield. *R-Stereochemistry not confirmed.

Formula: C₂₃H₂₉NO₇ Exact Mass: 431.19 Molecular Weight: 431.48

Analytical data for 8: ¹H NMR (400 MHz, methanol-d₄) δ ppm 7.75-7.83 (m, 2H) 7.47-7.55 (m, 2H) 7.19-7.33 (m, 3H) 4.57 (d, J=7.4 Hz, 1H) 4.22 (d, J=2.0 Hz, 1H) 3.89-3.96 (m, 2H) 3.63-3.71 (m, 4H) 3.26 (s, 3H) 2.93 (s, 3H) 2.27 (s, 3H); ESI-MS [M+H]⁺ calcd for C₂₃H₂₉NO₇H⁺432.20 found 432.4, (400.4 M-32+H), (863.7 2M+H).

### EXAMPLE 9

### 4′-(difluoro((2S,3S,4S,5S,6R)-3,4,5-trihydroxy-6-(hydroxymethyl)tetrahydro-2H-pyran-2-yl)methyl)-N,3′-dimethylbiphenyl-3-carboxamide

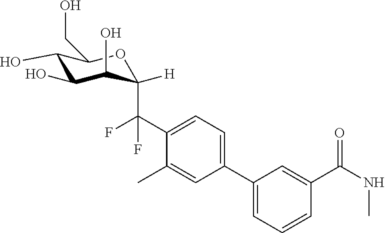

Following Scheme B, Intermediate 108 and commercially available N-methyl-3-(4,4,5,5-tetramethyl-[1,3,2]dioxaborolan-2-yl)-benzamide were reacted via the standard Suzuki coupling procedure (2 h at 80° C.), followed by deprotection protocol C (2 h at rt). The resulting residue was purified by HPLC (C18, 15*150 mm column; eluent: acetonitrile/water (0.05% TFA) to give 9 in 79% yield.

Formula: C₂₂H₂₅F₂NO₆ Exact Mass: 437.16 Molecular Weight: 437.43

Analytical data for 9: ¹H NMR (400 MHz, methanol-d₄) δ ppm 8.10 (s, 1H) 7.82 (dt, J=7.6, 0.9 Hz, 2H) 7.61 (d, J=2.7 Hz, 4H) 4.46 (dd, J=20.0, 12.0 Hz, 1H) 4.24 (br. s, 1H) 3.91 (br. s., 1H) 3.62-3.76 (m, 4H) 2.95 (s, 3H) 2.55 (s, 3H); ESI-MS [M+H]⁺ calcd for C₂₂H₂₅F₂NO₆H⁺438.17 found 438.4, (875.7 2M+H).

### EXAMPLE 10

### (2R,3S,4S,5S,6R)-2-((R)-Hydroxy (4-(isoquinolin-6-yl)-2-methylphenyl)methyl)-6-(hydroxymethyl)-tetrahydro-2H-pyran-3,4,5-triol

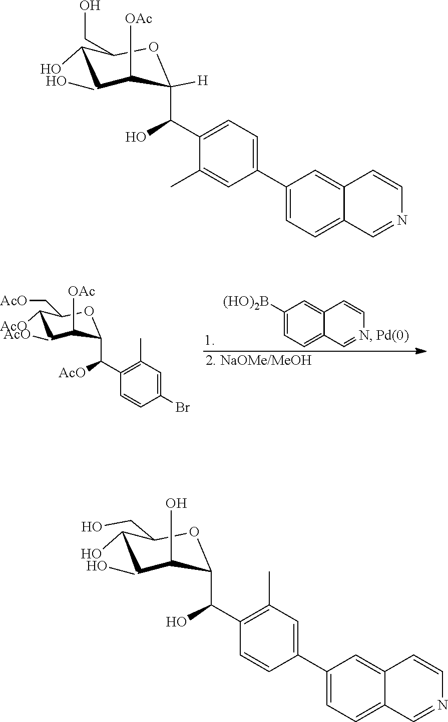

Following Scheme A, Intermediate 104R and commercially available isoquinolin-6-ylboronic acid were reacted via the standard Suzuki coupling procedure (40 min at 80° C.), followed first by deprotection protocol A (2 h at rt) then by purification using prep-HPLC with conditions: column: XBridge Prep C18 OBD Column 19×150 mm, 5 μm; mobile phase A: water with 0.05% NH₄HCO₃, mobile phase B: CH₃CN; flow rate: 20 mL/min; gradient: 2% B to 30% B in 15 min; 254 nm, RT 8 min to afford the title compound (38.8 mg, 36% yield) as a white solid.

Formula: C₂₃H₂₅NO₆ Exact Mass: 411.17 Molecular Weight: 411.45.

Analytical data: ¹H NMR (400 MHz, Methanol-d₄) 9.23 (s, 1H), 8.43 (d, J=5.6 Hz, 1H), 8.18-8.16 (m, 2H), 8.00 (d, J=8.0 Hz, 1H), 7.89 (d, J=5.6 Hz, 1H), 7.70-7.62 (m, 3H), 5.27 (d, J=6.8 Hz, 1H), 4.26 (t, J=2.8 Hz, 1H), 4.14 (dd, J=6.8 Hz, 2.0 Hz, 1H), 4.06 (dd, J=7.6 Hz, 3.2 Hz, 1H), 3.72-3.67 (m, 4H), 2.54 (s, 3H). ESI-MS [M+H]⁺ calcd for (C₂₃H₂₅NO₆H⁺) 412.2, found 412.2.

### EXAMPLE 11

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-methylisoquinolin-1 (2H)-one

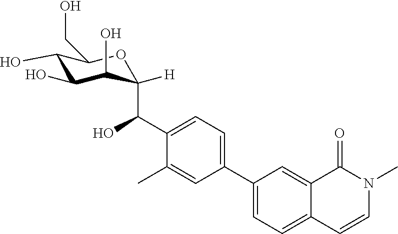

Step 1

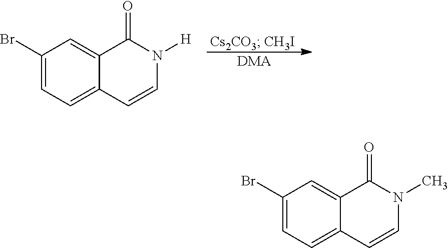

### 7-Bromo-2-methylisoquinolin-1 (2H)-one

To a solution of 7-bromoisoquinolin-1 (2H)-one (500 mg, 2.23 mmol, 1.0 equiv) and Cs₂CO₃ (1.1 g, 3.35 mmol, 1.5 equiv) in DMA (10 m L) was added CH₃I (475 mg, 3.35 mmol, 1.5 equiv) at rt. The mixture was stirred at 50° C. for 3 hours. Upon completion, the reaction was cooled to rt and diluted with water (50 mL). The mixture was extracted with EtOAc (3×10 mL). The combined organic layers were washed with water (2×10 mL) and saturated brine (10 mL), and dried over anhydrous Na₂SO₄. The filtrate was concentrated under reduced pressure. The residue was purified by silica gel chromatography, eluting with EtOAc in PE (0˜30%) to afford the title compound (470 mg, 88% yield) as a light yellow solid.

¹H NMR (300 MHz, CDCl₃) δ: 8.57 (d, J=2.1 Hz, 1H), 7.70 (dd, J=8.4 Hz, 2.1 Hz, 1H), 7.38 (d, J=8.4 Hz, 1H), 7.08 (d, J=7.2 Hz, 1H), 6.44 (d, J=7.5 Hz, 1H), 3.60 (s, 3H). ESI-MS [M+H]⁺ calcd for (C₁₀H₈BrNOH⁺) 238.1, found 237.8, 239.8.

Step 2

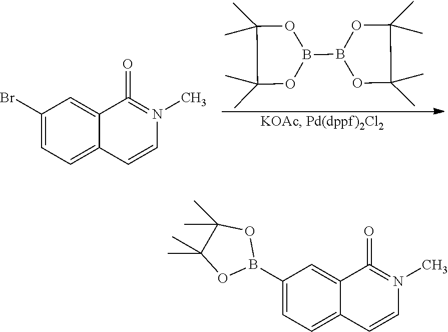

### 2-Methyl-7-(4,4,5,5-tetramethyl-1,3,2-dioxaborolan-2-yl)isoquinolin-1 (2H)-one

To a solution of the product from the previous step in dioxane (4 mL) was added bis(pinacolato)diboron (562 mg, 2.2 mmol, 1.1 equiv), Pd (dppf)Cl₂ (172 mg, 0.2 mmol, 0.1 equiv) and KOAc (592 mg, 6.0 mmol, 3.0 equiv) at rt. The resulting mixture was degassed and flushed with N₂ for three times and stirred for 1 h at 80° C. After completion, the reaction was cooled to rt and concentrated under reduced pressure. The residue was purified by silica gel chromatography, eluting with EtOAc in PE (0˜30%) to afford the title compound (480 mg, 85% yield) as a light brown solid.

MS (ESI+) calcd for (C₁₆H₂₀BNO₃H⁺) [M+H]⁺ 286.2, found 286.1.

Step 3

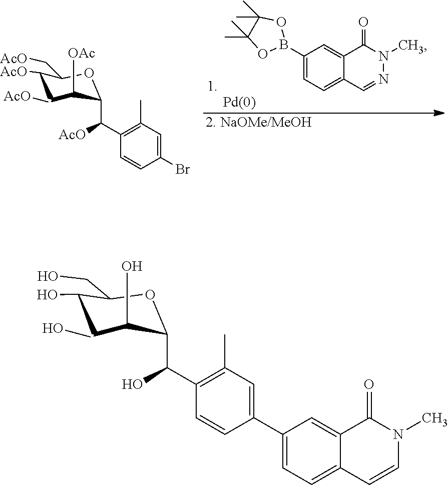

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-methylisoquinolin-1 (2H)-one

Following Scheme A, Intermediate 104R and the product from the previous step were reacted via the standard Suzuki coupling procedure (40 min at 80° C.), followed first by deprotection protocol A (2 h at rt) and then by purification using prep-HPLC with conditions: column: XBridge Prep C18 OBD Column 19×150 mm, 5 μm; mobile phase A: water with 0.05% NH₄HCO₃, mobile phase B: CH₃CN; flow rate: 20 mL/min; gradient: 2% B to 30% B in 15 min; 254 nm; Rt: 13.7 min to afford the title compound (43 mg, 37% yield for two steps) as a white solid.

Formula: C₂₄H₂₇NO₇ Exact Mass: 441.18 Molecular Weight: 441.47.

Analytical data: ¹H NMR (400 MHz, Methanol-d₄) 8.56 (d, J=1.6 Hz, 1H), 8.00 (dd, J=8.4, 2 Hz, 1H), 7.72 (d, J=8.4 Hz, 1H), 7.66-7.55 (m, 3H), 7.38 (d, J=7.2 Hz, 1H), 6.73 (d, J=7.2 Hz, 1H), 5.26 (d, J=6.8 Hz, 1H), 4.26 (t, J=3.2 Hz, 1H), 4.11 (2dd, J=4.8 Hz, 1.8 Hz, 1H), 4.05 (dd, J=8.0 Hz, 3.2 Hz, 1H), 3.73-3.62 (m, 7H), 2.52 (s, 3H). ESI-MS [M+H]⁺ calcd for (C₂₄H₂₇NO₇H⁺) 442.2, found 442.2.

### EXAMPLE 12

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-methyl-3,4-dihydroisoquinolin-1 (2H)-one

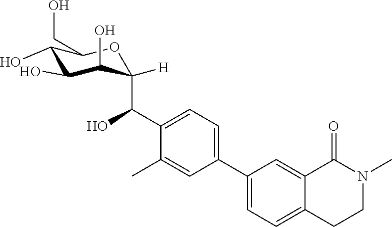

Step 1

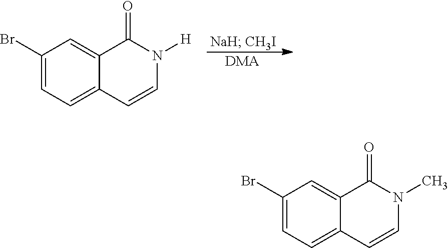

### 7-Bromo-2-methyl-3,4-dihydroisoquinolin-1 (2H)-one

To a solution of 7-bromo-3,4-dihydroisoquinolin-1 (2H)-one (300 mg, 1.33 mmol, 1.0 equiv) in DMA (5 mL) was added NaH (58.5 mg, 1.46 mmol, 1.1 equiv) at 0° C. The mixture was stirred at 0° C. for 30 min, then CH₃I (226 mg, 1.59 mmol, 1.2 equiv) was added to the mixture. The mixture was stirred at 0° C. for 2 hours. Upon completion, the reaction mixture was poured into water (20 mL) and was extracted with EtOAc (3×20 mL). The combined organic layers were washed with water (2×10 mL) and saturated brine (20 mL), and dried over anhydrous Na₂SO₄. The filtrate was concentrated under reduced pressure. The residue was purified by silica gel chromatography, eluting with EtOAc in PE (0˜30%) to afford the title compound (240 mg, 75% yield) as a light yellow solid.

ESI-MS [M+H]⁺ calcd for (C₁₀H₁₀BrNOH⁺) 240.0, found 239.9, 241.9.

Step 2

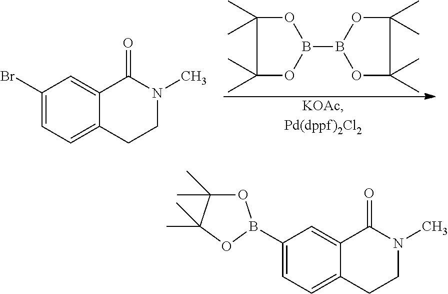

### 2-Methyl-7-(4,4,5,5-tetramethyl-1,3,2-dioxaborolan-2-yl)-3,4-dihydroisoquinolin-1 (2H)-one

To a solution of the product from the previous step (240 mg, 1.0 mmol, 1.0 equiv) in dioxane (4 mL) were added bis(pinacolato)diboron (279 mg, 1.1 mmol, 1.1 equiv), Pd(dppf)Cl₂ (81.6 mg, 0.1 mmol, 0.1 equiv) and KOAc (294 mg, 3.0 mmol, 3.0 equiv) at rt. The resulting mixture was degassed with N₂ for three times and stirred for 1 h at 80° C. After completion, the reaction was cooled to rt and concentrated under reduced pressure. The residue was purified by silica gel chromatography, eluting with EtOAc in PE (0˜30%) to afford the title compound (180 mg, 62% yield) as a light brown solid.

MS (ESI+) calcd for (C₁₆H₂₂BNO₃) [M+H]⁺ 288.2, found 288.2.

Step 3

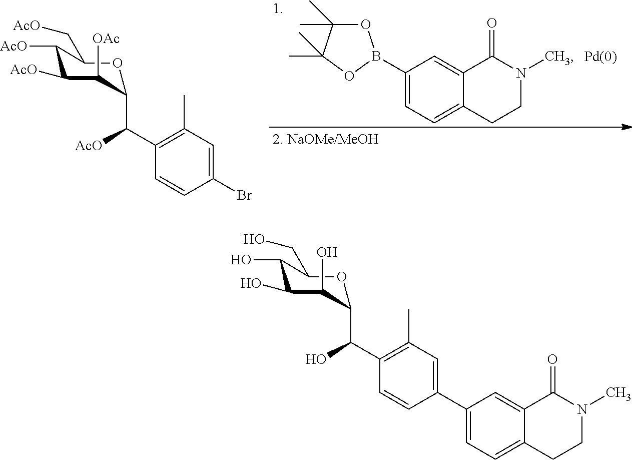

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-methyl-3,4-dihydroisoquinolin-1 (2H)-one

Following Scheme A, Intermediate 104R and the product from the previous step were reacted via the standard Suzuki coupling procedure (40 min at 80° C.), followed first by deprotection protocol A (2 h at rt), and then by purification using Prep-HPLC with conditions: column: XBridge Prep C18 OBD 100 Å, Column 19×250 mm, 10 am; mobile phase A: water with 0.05% NH₄HCO₃, mobile phase B: CH₃CN; flow rate: 20 mL/min; gradient: 20% B to 50% B in 15 min; 254/220 nm; Rt: 14.23 min to afford (50.7 mg, 44% for two steps) of the title compound as a white solid.

Formula: C₂₄H₂₉NO₇ Exact Mass: 443.19 Molecular Weight: 443.49.

Analytical data: ¹H NMR (400 MHz, Methanol-d₄) 8.19 (d, J=2.0 Hz, 1H), 7.74 (dd, J=7.6, 2.0 Hz, 1H), 7.61 (2d, J=8.0 Hz, 1H), 7.50 (dd, J=8.0, 2.0 Hz, 1H), 7.46 (d, J=2.0 Hz, 1H), 7.35 (d, J=8.0 Hz, 1H), 5.24 (d, J=6.8 Hz, 1H), 4.24 (t, J=3.2 Hz, 1H), 4.10 (dd, J=6.8 Hz, 2.4 Hz, 1H), 4.05 (dd, J=8.0 Hz, 3.2 Hz, 1H), 3.74-3.63 (m, 6H), 3.18 (s, 3H), 3.07 (t, J=6.4 Hz, 2H), 2.50 (s, 3H). ESI-MS [M+H]⁺ calcd for (C₂₄H₂₉NO₇H⁺) 444.2, found 444.2.

### EXAMPLE 13

### 4-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)isoindolin-1-one

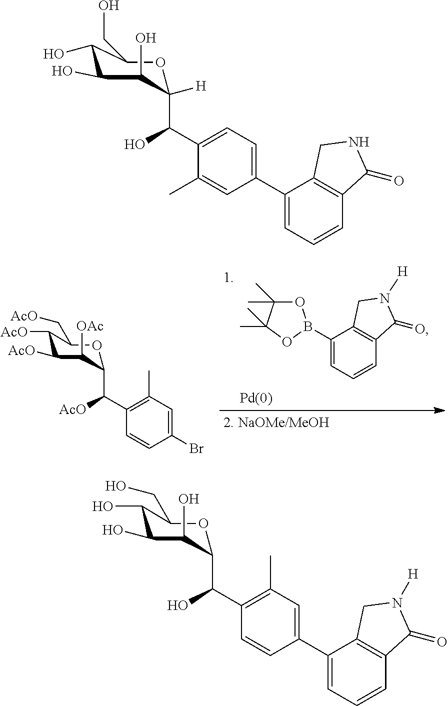

Following Scheme A, Intermediate 104R and commercially available 4-(4,4,5,5-tetramethyl-1,3,2-dioxaborolan-2-yl)isoindolin-1-one were reacted via the standard Suzuki coupling procedure (40 min at 80° C.), followed first by deprotection protocol A (2 h at rt), and then by purification using Prep-HPLC with conditions: column XBridge Prep C18 OBD Column, 100 Å, 19×250 mm, 5 μm; mobile phase A: water with 0.05% NH₄HCO₃, mobile phase B: CH₃CN; flow rate: 20 mL/min; gradient: 35% B to 55% B in 7 min; 254 nm; RT 6.45 min to afford the title compound (73.9 mg, 68% for two steps) as a white solid.

Formula: C₂₂H₂₅NO₇ Exact Mass: 415.16 Molecular Weight: 415.44.

Analytical data: ¹H NMR (400 MHz, Methanol-d₄) 7.79 (dd, J=7.2, 1.2 Hz, 1H), 7.66-7.58 (m, 3H), 7.40 (dd, J=8.0 Hz, 1.6 Hz, 1H), 7.35 (d, J=1.6 Hz, 1H), 5.25 (d, J=6.8 Hz, 1H), 4.55 (s, 2H), 4.25 (t, J=2.8 Hz, 1H), 4.11 (2dd, J=6.8 Hz, 2.8 Hz, 1H), 4.04 (dd, J=8.0, 2.8 Hz, 1H), 3.72-3.66 (m, 4H), 2.50 (s, 3H). ESI-MS [M+H]⁺ calcd for (C₂₂H₂₅NO₇H⁺) 416.2, found 416.2.

### EXAMPLE 14

### (2R,3S,4S,5S,6R)-2-((R)-Hydroxy (2-methyl-4-(quinolin-6-yl)phenyl)methyl)-6-(hydroxymethyl)-tetrahydro-2H-pyran-3,4,5-triol

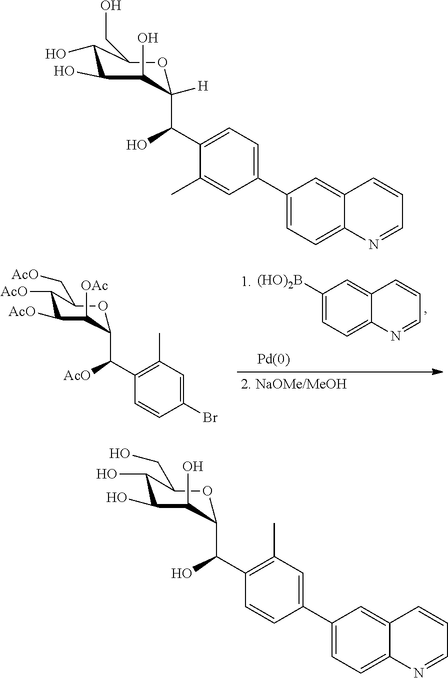

Following Scheme A, Intermediate 104R and commercially available quinolin-6-ylboronic acid were reacted via the standard Suzuki coupling procedure (30 min at 80° C.), followed first by deprotection protocol A (2 h at rt), and then by purification using Prep-HPLC with conditions: column: XBridge Prep OBD C18 Column 30×150 mm 5 um; mobile phase A: Water (10 mmol/L NH₄HCO₃), mobile phase B: CH₃CN; flow rate: 20 mL/min; gradient: 2% B to 25% B in 14 min; 254 nm; Rt: 13.5 min to give the title compound (26.3 mg, 29% yield for two steps) as a white solid.

Formula: C₂₃H₂₅NO₆ Exact Mass: 411.17 Molecular Weight: 411.45.

Analytical data: ¹H NMR (400 MHz, Methanol-d₄) 8.83 (dd, J=4.4 Hz, 1.6 Hz, 1H), 8.44 (dd, J=8.4, 1.6 Hz, 1H), 8.18 (m, 1H), 8.09 (d, J=1.2 Hz, 2H), 7.68-7.63 (m, 2H), 7.61 (2s, 1H), 7.56 (dd, J=8.0 Hz, 4.0 Hz, 1H), 5.26 (d, J=6.8 Hz, 1H), 4.26 (t, J=3.2 Hz, 1H), 4.12 (dd, J=6.8 Hz, 2.8 Hz, 1H), 4.07-4.04 (m, 1H), 3.72-3.70 (m, 2H), 3.68-3.64 (m, 2H), 2.54 (s, 3H). ESI-MS [M+H]⁺ calcd for (C₂₃H₂₅NO₆H⁺) 412.18, found 412.4.

### EXAMPLE 15

### (2R,3S,4S,5S,6R)-2-((R)-Hydroxy (2-methyl-4-(quinolin-7-yl)phenyl)methyl)-6-(hydroxymethyl)-tetrahydro-2H-pyran-3,4,5-triol

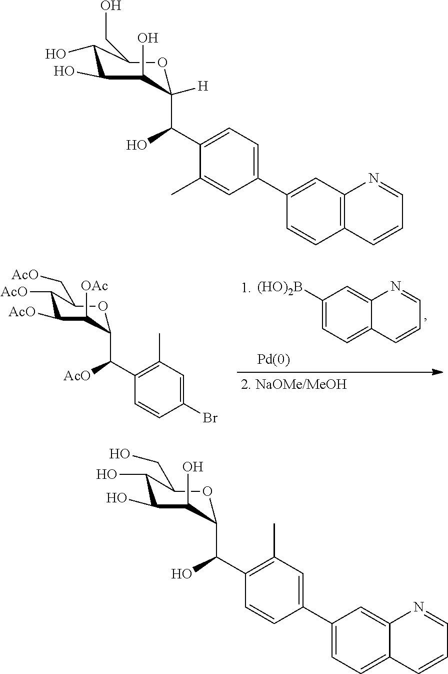

Following Scheme A, Intermediate 104R and commercially available 7-(4,4,5,5-tetramethyl-1,3,2-dioxaborolan-2-yl)quinoline were reacted via the standard Suzuki coupling procedure (30 min at 80° C.), followed first by deprotection protocol A (2 h at rt), and then by purification using Prep-HPLC with conditions: column: XBridge Prep OBD C18 Column 30×150 mm 5 um; mobile phase A: Water (10 mmol/L NH₄HCO₃), mobile phase B: CH₃CN; flow rate: 60 mL/min; gradient: 10% B to 25% B in 10 min; 254 nm; Rt: 10.27 min to give the title compound (19.6 mg, 30% yield for two steps) as a white solid.

Formula: C₂₃H₂₅NO₆ Exact Mass: 411.17 Molecular Weight: 411.45.

Analytical data: ¹H NMR (300 MHz, DMSO-d6+D₂O) δ ppm 8.88 (dd, J=4.2 Hz, 1.5 Hz, 1H), 8.37 (d, J=8.4 Hz, 1H), 8.22 (s, 1H), 8.05 (t, J=8.7 Hz, 1H), 7.93 (dd, J=8.7 Hz, 1.5 Hz, 1H), 7.65 (dd, J=8.1 Hz, 1.5 Hz, 1H), 7.60 (s, 1H), 7.55-7.50 (m, 2H), 5.03 (d, J=7.2 Hz, 1H), 4.04 (t, J=2.7 Hz, 1H), 3.88 (dd, J=7.2 Hz, 2.4 Hz, 1H), 3.47-3.43 (m, 4H), 2.42 (s, 3H) ESI-MS [M+H]⁺ calcd for (C₂₃H₂₅NO₆H⁺) 412.18, found 412.2.

### EXAMPLE 16

### (2R,3S,4S,5S,6R)-2-((R)-Hydroxy (2-methyl-4-(3-methyl-3H-benzo[d]imidazol-5-yl)phenyl)methyl)-6-(hydroxymethyl)-tetrahydro-2H-pyran-3,4,5-triol

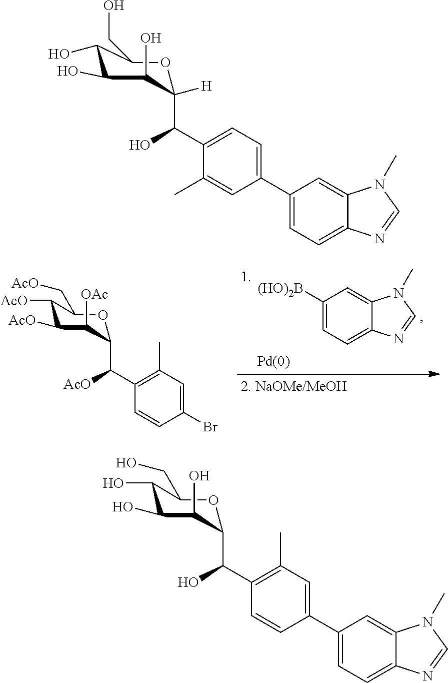

Following Scheme A, Intermediate 104R and commercially available 3-methyl-3H-benzo[d]imidazol-5-ylboronic acid were reacted via the standard Suzuki coupling procedure (1.5 h at 80° C.), followed first by deprotection protocol A (2 h at rt), and then by purification using Prep-HPLC with conditions: column: Atlantis Prep T3 OBD Column, 19*250 mm 10 um; mobile phase A: Water (0.05% TFA), mobile phase B: CH₃CN; flow rate: 20 mL/min; gradient: 10% B to 30% B in 11 min; 254/220 nm; Rt: 7.83 min to afford the title compound (33 mg, 23% for two steps) as a white solid.

Formula: C₂₂H₂₆N₂O₆ Exact Mass: 414.18 Molecular Weight: 414.45.

Analytical data: ¹H NMR (300 MHz, Methanol-d₄) δ 9.33 (s, 1H), 8.14 (s, 1H), 8.00-7.87 (m, 2H), 7.68-7.57 (m, 3H), 5.26 (d, J=6.9 Hz, 1H), 4.25 (t, J=2.7 Hz, 1H), 4.19 (s, 3H), 4.12 (dd, J=6.9 Hz, 2.7 Hz, 1H), 4.05-4.02 (m, 1H), 3.71-3.65 (m, 4H), 2.54 (s, 3H). ESI-MS [M+H]⁺ calcd for (C₂₂H₂₆N₂O₆H⁺) 415.19, found 415.05.

### EXAMPLE 17

### (2R,3S,4S,5S,6R)-2-((R)-(4-(3H-Benzo[d]imidazol-5-yl)-2-methylphenyl) (hydroxy)methyl)-6-(hydroxymethyl)-tetrahydro-2H-pyran-3,4,5-triol

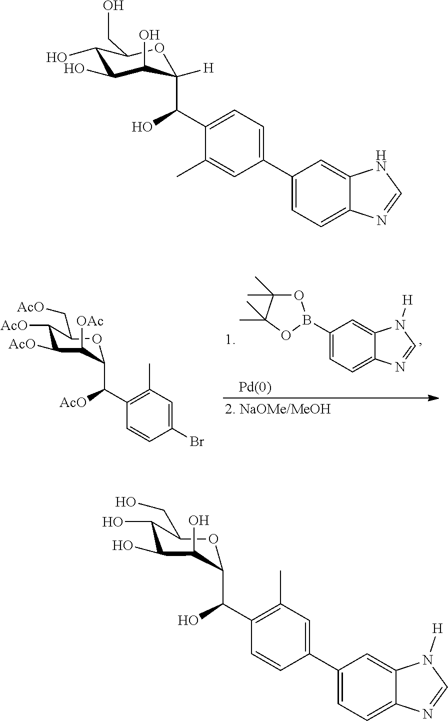

Following Scheme A, Intermediate 104R and commercially available 6-(4,4,5,5-tetramethyl-1,3,2-dioxaborolan-2-yl)-1H-benzo[d]imidazole were reacted via the standard Suzuki coupling procedure (1.5 h at 80° C.), followed first by deprotection protocol A (2 h at rt), and then by purification using Prep-HPLC with conditions: column: Atlantis Prep T3 OBD Column, 19*250 mm, 10 u; mobile phase A: Water (0.05% TFA), mobile phase B: CH₃CN; flow rate: 20 mL/min; gradient: 5% B to 25% B in 11 min; 254/220 nm; Rt: 9.58 min to afford the title compound (16 mg, 10% yield for two steps) as a white solid.

Formula: C₂₁H₂₄N₂O₆ Exact Mass: 400.16 Molecular Weight: 400.43.

Analytical data: ¹H NMR (300 MHz, Methanol-d₄) δ 9.21-9.15 (m, 1H), 7.98 (s, 1H), 7.85 (d, J=3.3 Hz, 2H), 7.64 (d, J=8.1 Hz, 1H), 7.56-7.51 (2m, 2H), 5.24 (d, J=6.9 Hz, 1H), 4.25 (t, J=3.3 Hz, 1H), 4.11 (2dd, J=6.9 Hz, 2.4 Hz, 1H), 4.05-4.01 (2m, 1H), 3.72-3.59 (m, 4H), 2.52 (s, 3H). ESI-MS [M+H]⁺ calcd for (C₂₁H₂₄N₂O₆H⁺) 401.17, found 401.15.

### EXAMPLE 18

### (2R,3S,4S,5S,6R)-2-((R)-(4-(3-Amino-1H-indazol-7-yl)-2-methylphenyl) (hydroxy)methyl)-6-(hydroxymethyl)-tetrahydro-2H-pyran-3,4,5-triol

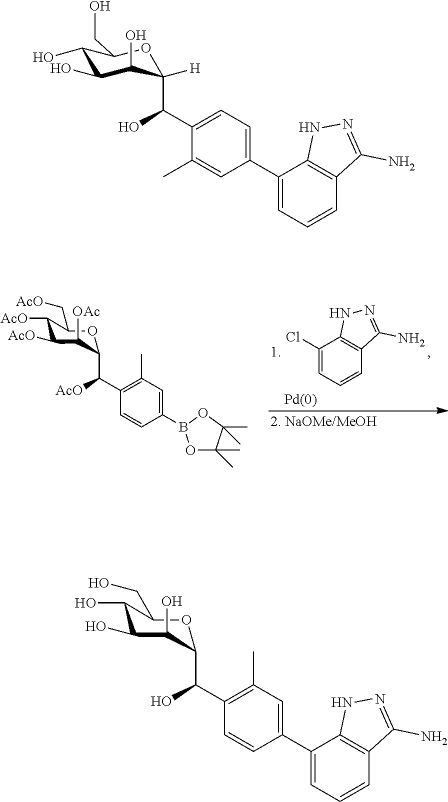

Following Scheme C, to a solution of Intermediate 105R (200 mg, 0.32 mmol) in dioxane/water (10 mL/2 mL) were added 7-chloro-1H-indazol-3-amine (59 mg, 0.35 mmol), K₃PO₄ (136 mg, 0.78 mmol) and 2^{nd} Generation SPhos precatalyst (11.5 mg, 0.016 mmol) at rt. The resulting mixture was degassed three times with N₂ and stirred at 100° C. for 1 hour. Upon completion, the reaction was cooled to rt and concentrated under reduced pressure. The residue was purified by silica gel chromatography, eluting with EtOAc in PE (0-100%) to afford the coupled product (180 mg, crude, contained some deacetylated compound) as a light yellow solid, The product was then subjected to deprotection protocol A (2 h at rt), followed by purification with Prep-HPLC with conditions: column: XBridge CSH Prep C18 OBD Column, 5 am, 19×150 mm, 5; mobile phase A: water with 0.05% TFA, mobile phase B: CH₃CN; flow rate: 20 mL/min; gradient: 5% B to 22% B in 7 min; 254 nm; Rt: 6.02 min to afford the title compound (75.4 mg, 37% for two steps) as an off-white solid.

Formula: C₂₁H₂₅N₃O₆ Exact Mass: 415.17 Molecular Weight: 415.44.

Analytical data: ¹H NMR (400 MHz, CD₃OD) δ: 7.88 (dd, J=8.4 Hz, 0.8 Hz, 1H), 7.68-7.64 (m, 2H), 7.51 (2dd, J=8.0 Hz, 1.6 Hz, 1H), 7.47 (s, 1H), 7.33 (dd, J=8.0, 7.6 Hz, 1H), 5.26 (d, J=6.8 Hz, 1H), 4.25 (t, J=3.2 Hz, 1H), 4.13 (dd, J=7.2 Hz, 2.8 Hz, 1H), 4.04-4.01 (2m, 1H), 3.70-3.67 (m, 4H), 2.52 (s, 3H). ESI-MS [M+H]⁺ calcd for r (C₂₁H₂₅N₃O₆H⁺) 416.18, found 416.15.

### EXAMPLE 19

### 6-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)isoindolin-1-one

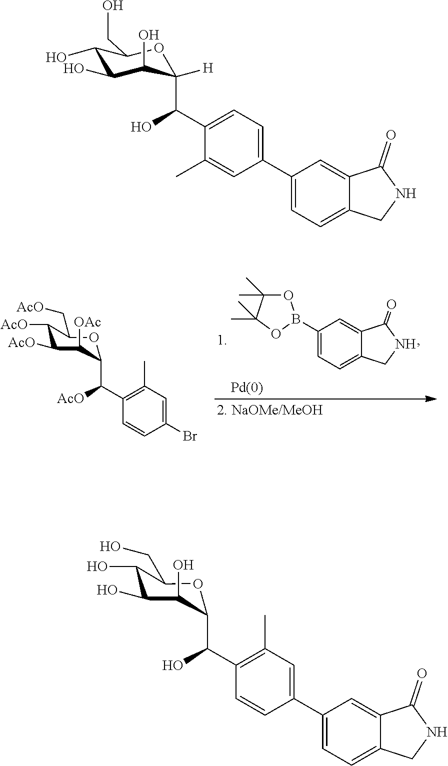

Following Scheme A, Intermediate 104R and commercially available 6-(4,4,5,5-tetramethyl-1,3,2-dioxaborolan-2-yl)isoindolin-1-one were reacted via the standard Suzuki coupling procedure (40 min at 80° C.), followed first by deprotection protocol A (2 h at rt), and then by purification using Prep-HPLC with conditions: column: XBridge Prep C18 OBD Column 19×150 mm, 5 μm; mobile phase A: water with 0.05% NH₄HCO₃, mobile phase B: CH₃CN; flow rate: 20 mL/min; gradient: 5% B to 35% B in 7 min; 254 nm; Rt 5.08 min to afford the title compound (48.5 mg, 48% for two steps) as a white solid.

Formula: C₂₂H₂₅NO₇ Exact Mass: 415.16 Molecular Weight: 415.44.

Analytical data: ¹H NMR (400 MHz, CD₃OD) δ: 8.01 (2s, 1H), 7.89 (dd, J=8.0 Hz, 1.6 Hz, 1H), 7.66-7.62 (m, 2H), 7.52 (d, J=8.0 Hz, 1.6 Hz, 1H), 7.48 (s, 1H), 5.25 (d, J=6.8 Hz, 1H), 4.51 (2s, 2H), 4.25 (t, J=3.2 Hz, 1H), 4.10 (dd, J=6.8 Hz, 2.4 Hz, 1H), 4.05 (dd, J=8.0 Hz, 3.2 Hz, 1H), 3.74-3.66 (m, 4H), 2.51 (2s, 3H). ESI-MS [M+Na]⁺ calcd for (C₂₂H₂₅NO₇Na⁺⁾ 438.17, found 438.20.

### EXAMPLE 20

### (2R,3S,4S,5S,6R)-2-((R)-Hydroxy (2-methyl-4-(3-methylbenzo[d]isoxazol-5-yl)phenyl)methyl)-6-(hydroxymethyl)-tetrahydro-2H-pyran-3,4,5-triol

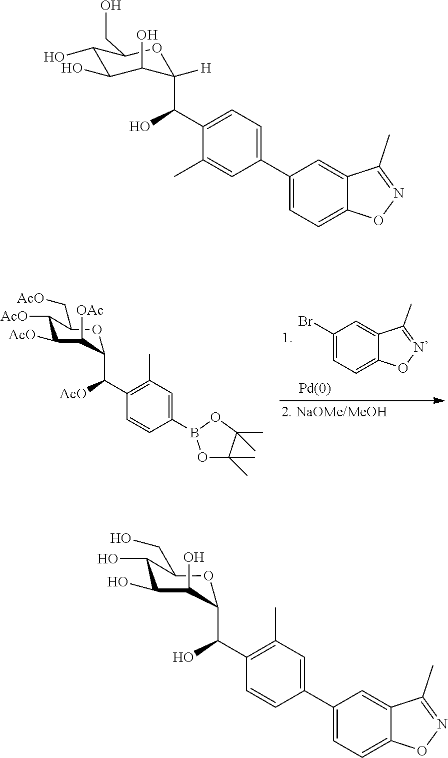

Following Scheme C, Intermediate 105R and commercially available 5-bromo-3-methylbenzo[d]isoxazole were reacted via the standard Suzuki coupling procedure (40 min at 80° C.), followed first by deprotection protocol A (2 h at rt), and then by purification using Prep-HPLC with conditions: column: XBridge CSH Prep C18 OBD Column, 5 μm, 19×150 mm; mobile phase A: water with 0.05% TFA, mobile phase B: ethyl alcohol; flow rate: 20 mL/min; gradient: 20% B to 55% B in 7 min; 254 nm; Rt: 4.85 min to afford the title compound (13.8 mg, 14% for two steps) as a white solid.

Formula: C₂₂H₂₅NO₇ Exact Mass: 415.16 Molecular Weight: 415.44.

Analytical data: ¹H NMR (400 MHz, CD₃OD) δ: 7.92 (d, J=2.4 Hz, 1H), 7.54 (d, J=8.0 Hz, 1H), 7.40 (dd, J=8.0 Hz, 1.6 Hz, 1H), 7.36 (s, 1H), 7.27 (dd, J=8.4 Hz, 2.0 Hz, 1H), 6.92 (d, J=8.4 Hz, 1H), 5.22 (d, J=6.8 Hz, 1H), 4.24 (t, J=2.8 Hz, 1H), 4.09 (dd, J=6.8 Hz, 2.4 Hz, 1H), 4.05-4.02 (m, 1H), 3.70-3.65 (m, 4H), 2.47 (s, 3H), 2.20 (s, 3H). ESI-MS [M+H]⁺ calcd for (C₂₂H₂₅NO₇H⁺) 416.17, found 416.10.

### EXAMPLE 21

### (2R,3S,4S,5S,6R)-2-((R)-Hydroxy (4-(isoquinolin-7-yl)-2-methylphenyl)methyl)-6-(hydroxymethyl)-tetrahydro-2H-pyran-3,4,5-triol

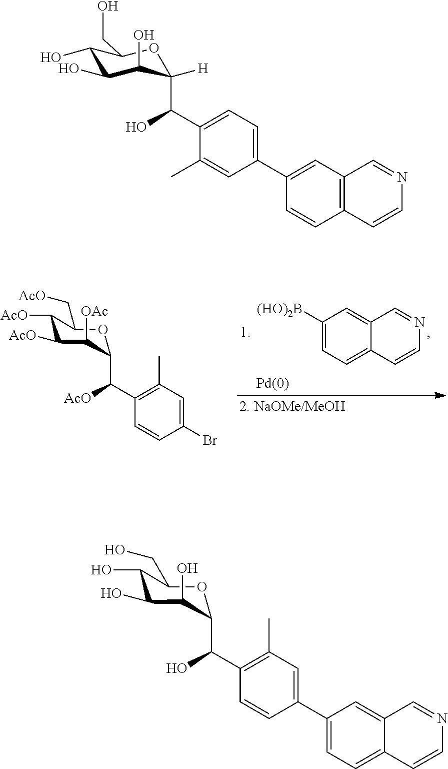

Following Scheme A, Intermediate 104R and commercially available isoquinolin-7-ylboronic acid were reacted via the standard Suzuki coupling procedure (40 min at 80° C.), followed first by deprotection protocol A (2 h at rt), and then by purification using Prep-HPLC with conditions: column: XBridge Prep C18 OBD Column, 100 Å, 19×250 mm, 5 m; mobile phase A: water with 0.05% NH₄HCO₃, mobile phase B: CH₃CN; flow rate: 20 mL/min; gradient: 20% B to 45% B in 15 min; 254 nm; Rt: 12.35 min to afford the title compound (33.4 mg, 31% for two steps) as a white solid.

Formula: C₂₃H₂₅NO₆ Exact Mass: 411.17 Molecular Weight: 411.45.

Analytical data: ¹H NMR (400 MHz, Methanol-d₄) 9.30 (s, 1H), 8.42 (d, J=6.0 Hz, 1H), 8.34 (s, 1H), 8.11 (2dd, J=8.8, 1.6 Hz, 1H), 8.02 (d, J=8.4 Hz, 1H), 7.84 (d, J=6.0 Hz, 1H), 7.69-7.61 (2m, 3H), 5.27 (d, J=6.8 Hz, 1H), 4.26 (t, J=2.8 Hz, 1H), 4.13 (dd, J=6.4 Hz, 2.8 Hz, 1H), 4.07-4.04 (m, 1H), 3.72-3.67 (m, 4H), 2.54 (s, 3H). ESI-MS [M+H]⁺ calcd for (C₂₃H₂₅NO₆H⁻) 412.18, found 412.10.

### EXAMPLE 22

### (2R,3S,4S,5S,6R)-2-((R)-(4-(2-Chloropyridin-4-yl)-2-methylphenyl) (hydroxy)methyl)-6-(hydroxymethyl)-tetrahydro-2H-pyran-3,4,5-triol

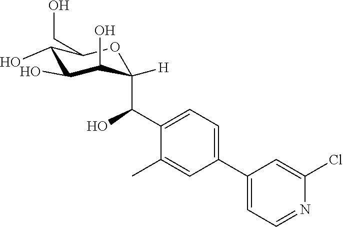

### EXAMPLE 23

### (2R,3S,4S,5S,6R)-2-((R)-(4-(2′-Chloro-[2,4′-bipyridin]-4-yl)-2-methylphenyl) (hydroxy)methyl)-6-(hydroxymethyl)tetrahydro-2H-pyran-3,4,5-triol

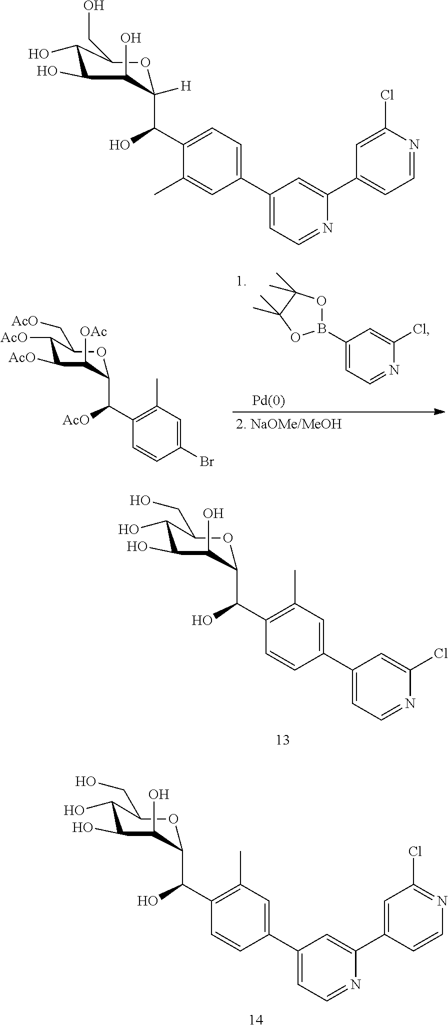

Following Scheme A, Intermediate 104R and commercially available 2-chloro-4-(4,4,5,5-tetramethyl-1,3,2-dioxaborolan-2-yl)pyridine were reacted via the standard Suzuki coupling procedure (1.5 h at 80° C.), followed first by deprotection protocol A (2 h at rt), and then by purification and separation of Example 22 and Example 23 using Prep-HPLC with conditions: column: Atlantis Prep T3 OBD Column, 19*250 mm 10 um; mobile phase A: Water (0.05% TFA), mobile phase B: CH₃CN; flow rate: 20 mL/min; gradient: 15% B to 15% B in 36 min; 254/220 nm; Rt: 32.57 min to afford Example 22 (retention time: 36 min, 16 mg, 13% for two steps) TFA salt as a white salt and Example 23 (retention time 32.57 min, 8 mg, 13% for two steps) TFA salt as a white salt.

Example 22 Formula: C₁₉H₂₂C₁NO₆ Exact Mass: 395.11 Molecular Weight: 395.83.

Example 22 Analytical data: ¹H NMR (300 MHz, Methanol-d₄) δ 8.38 (d, J=5.1 Hz, 1H), 7.75 (d, J=0.9 Hz, 1H), 7.69-7.57 (m, 4H), 5.24 (d, J=6.9 Hz, 1H), 4.21 (2t, J=3.0 Hz, 1H), 4.09 (dd, J=6.9 Hz, 2.7 Hz, 1H), 4.05-4.01 (2m, 1H), 3.70-3.65 (m, 4H), 2.52 (s, 3H).) ESI-MS [M+H]⁺ calcd for (C₁₉H₂₂C₁NO₆H) 396.12, found 396.10.

Example 23 Formula: C₂₄H₂₅C₁N₂O₆ Exact Mass: 472.14 Molecular Weight: 472.92.

Example 23 Analytical data: ¹H NMR (400 MHz, Methanol-d₄) δ 8.75 (d, J=5.2 Hz, 1H), 8.51 (2d, J=5.2 Hz, 1H), 8.30 (s, 1H), 8.20 (s, 1H), 8.08 (dd, J=5.2 Hz, 1.2 Hz, 1H), 7.80 (dd, J=5.2 Hz, 1.6 Hz, 1H), 7.72-7.70 (m, 3H), 5.27 (d, J=6.4 Hz, 1H), 4.23 (t, J=2.8 Hz, 1H), 4.11 (2dd, J=6.4 Hz, 2.8 Hz, 1H), 4.05-4.02 (m, 1H), 3.71-3.66 (m, 4H), 2.55 (s, 3H). ESI-MS [M+H]⁺ calcd for (C₂₄H₂₅C₁N₂O₆H⁺) 473.15, found 473.00.

### EXAMPLE 24

### (2R,3S,4S,5S,6R)-2-((R)-Hydroxy (2-methyl-4-(2-(methylamino)pyridin-4-yl)phenyl)methyl)-6-(hydroxymethyl)-tetrahydro-2H-pyran-3,4,5-triol

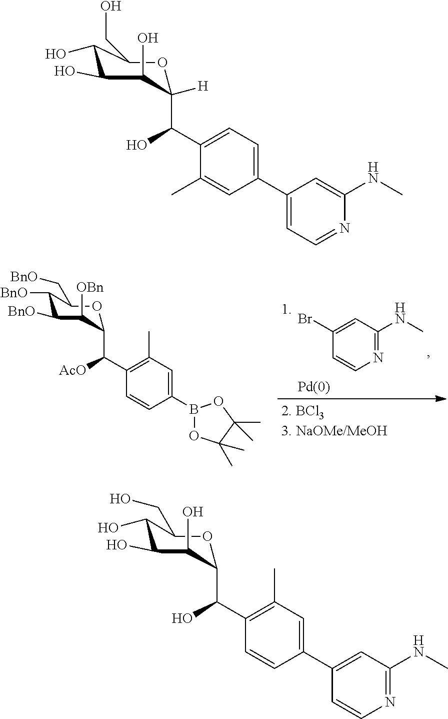

Following Scheme D, Intermediate 106R and commercially purchased 4-bromo-N-methylpyridin-2-amine were reacted via the standard Suzuki coupling procedure (1.5 h at 80° C.), followed first by deprotection protocol B (30 min at −78° C.), then by deprotection protocol A (2 h at rt), and then by purification using Prep-HPLC with conditions: column: Atlantis Prep T3 OBD Column, 19*250 mm 10 u; mobile phase A: Water (0.05% TFA), mobile phase B: CH₃CN; flow rate: 20 mL/min; gradient: 15% B to 43.6% B in 5 min; 254 nm; Rt: 3.93 min to afford the title compound (40 mg, 41% yield) as a white solid.

Formula: C₂₀H₂₆N₂O₆ Exact Mass: 390.18 Molecular Weight: 390.43.

Analytical data: ¹H NMR (300 MHz, Methanol-d₄) δ 7.87-7.85 (m, 1H), 7.71 (2d, J=8.1 Hz, 1H), 7.64-7.559 (m, 2H), 7.22-7.20 (m, 2H), 5.24 (d, J=6.6 Hz, 1H), 4.19 (t, J=3.3 Hz, 1H), 4.08 (dd, J=6.9 Hz, 3.0 Hz, 1H), 4.02-3.98 (m, 1H), 3.69-3.64 (m, 4H), 3.07 (s, 3H), 2.52 (s, 3H). ESI-MS [M+H]⁺ calcd for (C₂₀H₂₆N₂O₆H⁺) 391.19, found 391.15.

### EXAMPLE 25

### (2R,3S,4S,5S,6R)-2-((R)-Hydroxy (2-methyl-4-(2-morpholinopyridin-4-yl)phenyl)methyl)-6-(hydroxymethyl)-tetrahydro-2H-pyran-3,4,5-triol

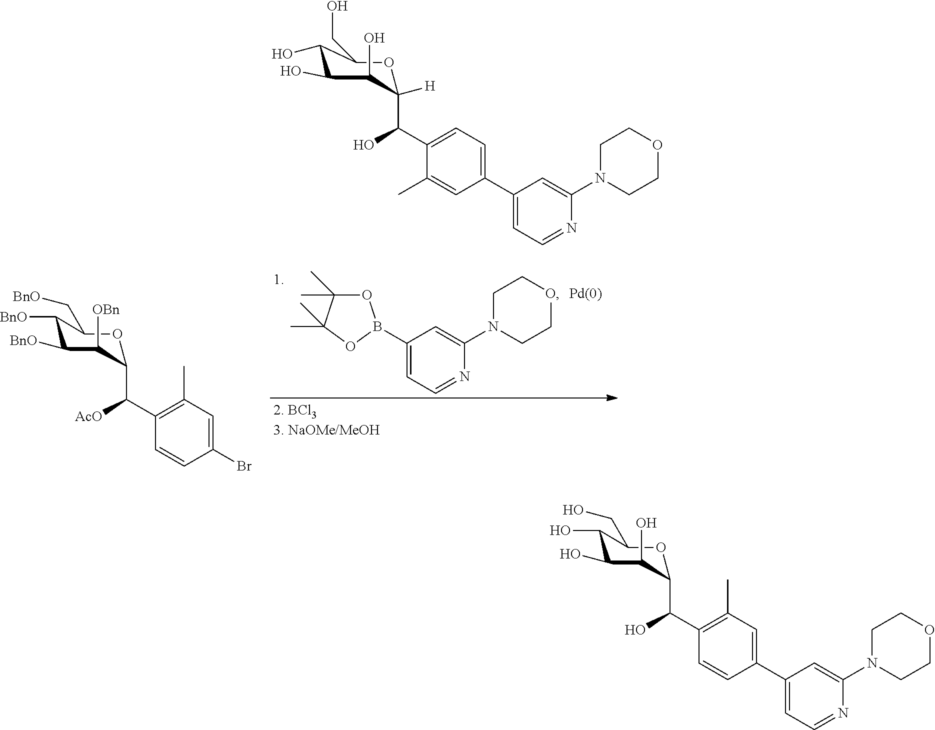

Following Scheme B, Intermediate 103R and commercially available 4-(4-(4,4,5,5-tetramethyl-1,3,2-dioxaborolan-2-yl)pyridin-2-yl)morpholine were reacted via the standard Suzuki coupling procedure (1.5 h at 80° C.), followed first by deprotection protocol B (30 min at −78° C.), then by deprotection protocol A (1 h at rt), and then by purification using Prep-HPLC with conditions: column XBridge Prep C18 OBD Column 19×150 mm 5 um; mobile phase A: Water (0.05% TFA), mobile phase B: CH₃CN; flow rate: 20 mL/min; gradient: 3% B to 30% B in 5 min; 254 nm; Rt: 4.12 min to afford the title compound (40 mg, 34% yield) as a light pink solid.

Formula: C₂₃H₃₀N₂O₇ Exact Mass: 446.21 Molecular Weight: 446.49.

Analytical data: ¹H NMR (300 MHz, Methanol-d₄) δ 8.14 (d, J=5.1, Hz, 1H), 7.64-7.50 (m, 3H), 7.01-6.97 (m, 2H), 5.23 (d, J=6.9 Hz, 1H), 4.23 (t, J=3.0, 1H), 4.09 (dd, J=6.6 Hz, 2.7 Hz, 1H), 4.05-4.01 (2m, 1H), 3.82 (t, J=4.8 Hz, 4H), 3.77-3.64 (m, 4H), 3.53 (t, J=4.5 Hz, 4H), 2.50 (s, 3H). ESI-MS [M+H]⁺ calcd for (C₂₃H₃₀N₂O₇H⁺⁾447.21, found 447.05.

### EXAMPLE 26

### (2R,3S,4S,5S,6R)-2-((R)-(4-(2-Aminopyridin-4-yl)-2-methylphenyl) (hydroxy)methyl)-6-(hydroxymethyl)-tetrahydro-2H-pyran-3,4,5-triol

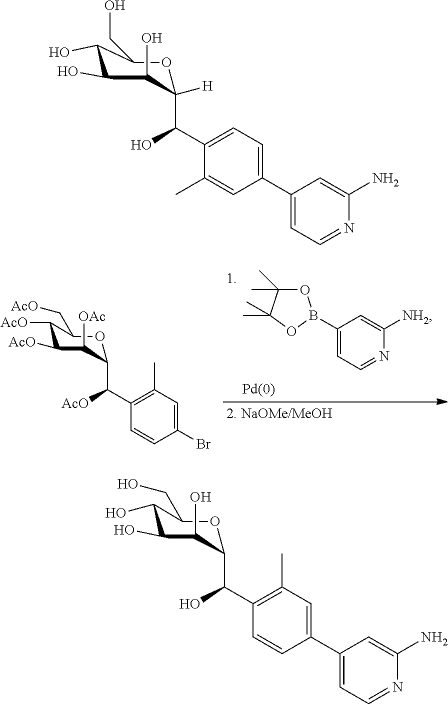

Following Scheme A, Intermediate 104R and commercially available 4-(4,4,5,5-tetramethyl-1,3,2-dioxaborolan-2-yl)pyridin-2-amine were reacted via the standard Suzuki coupling procedure (1.5 h at 80° C.), followed first by deprotection protocol A (2 h at rt) and then by purification using Prep-HPLC with conditions: column: XSelect CSH Prep C18 OBD Column, 5 um, 19*150 mm; mobile phase A: Water (0.05% TFA), column XSelect CSH Prep C18 OBD Column, 5 um, 19*150 mm; mobile phase A: Water (0.05% TFA), mobile phase B: CH₃CN; flow rate: 20 mL/min; gradient: 1% B to 12.1% B in 7 min; 254/220 nm; Rt: 6.67 min to afford the title compound (40.9 mg, 29% for two steps) as a white solid.

Formula: C₁₉H₂₄N₂O₆ Exact Mass: 376.16 Molecular Weight: 376.40.

Analytical data: ¹H NMR (400 MHz, Methanol-d₄) δ 7.87 (d, J=6.8 Hz, 1H), 7.71 (2d, J=8.0 Hz, 1H), 7.62 (dd, J=8.0 Hz, 1.6 Hz, 1H), 7.58 (d, J=1.6 Hz, 1H), 7.23-7.22 (m, 2H), 5.25 (d, J=6.8 Hz, 1H), 4.20 (t, J=3.2 Hz, 1H), 4.09 (dd, J=6.8 Hz, 3.2 Hz, 1H), 4.02-3.99 (m, 1H), 3.69-3.65 (m, 4H), 2.55 (s, 3H). ESI-MS [M+H]⁺ calcd for (C₁₉H₂₄N₂O₆H⁺), 377.17, found 377.15.

### EXAMPLE 27

### (2R,3S,4S,5S,6R)-2-((R)-(4-(2-(Dimethylamino)pyridin-4-yl)-2-methylphenyl) (hydroxy)methyl)-6-(hydroxymethyl)-tetrahydro-2H-pyran-3,4,5-triol

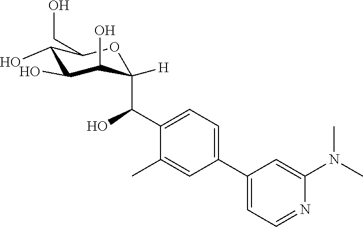

Step 1

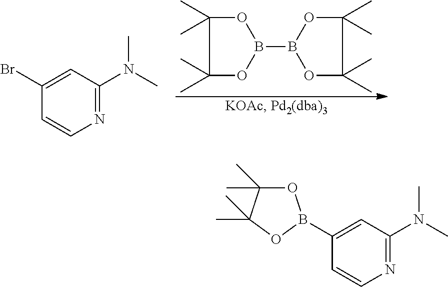

### N,N-Dimethyl-4-(4,4,5,5-tetramethyl-1,3,2-dioxaborolan-2-yl)pyridin-2-amine

To a solution of 3-bromo-N,N-dimethylaniline (320 mg, 1.60 mmol), bis(pinacolato)diboron (445 mg, 1.75 mmol) and KOAc (235 mg, 2.36 mmol) in dioxane (10.0 mL) was added Pd₂(dba)₃ (83 mg, 0.08 mmol) and PCy₃ (47 mg 0.128 mmol) under N₂, The resulting reaction mixture was heated to 85° C. for 3 h. After the completion, the reaction was cooled to rt and concentration in vacuo. The residue was purified by silica gel chromatography, eluting with EtOAc in PE (0-60%) to give crude title compound (˜90 mg) as a light brown solid.

ESI-MS calcd for (C₁₃H₂₁BN₂O₂H⁺) [M+H]⁺ 249.17, found 249.20.

Step 2

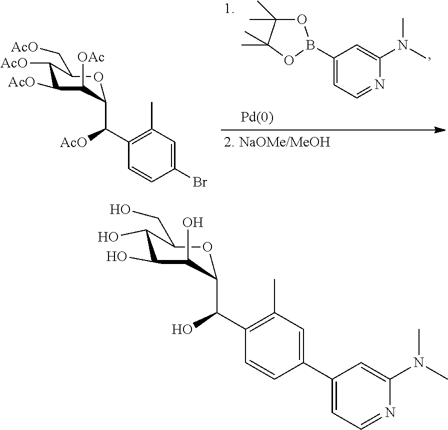

### (2R,3S,4S,5S,6R)-2-((R)-(4-(2-(Dimethylamino)pyridin-4-yl)-2-methylphenyl) (hydroxy)methyl)-6-(hydroxymethyl)-tetrahydro-2H-pyran-3,4,5-triol

Following scheme A, Intermediate 104R and the product from the previous step were reacted via the standard Suzuki coupling procedure (1.5 h at 80° C.), followed first by deprotection protocol A (2 h at rt), then by purification using Prep-HPLC with conditions: column XBridge Prep OBD C18 Column 30×150 mm 5 um; mobile phase A: Water (10 mmol/L NH₄HCO₃), mobile phase B: CH₃CN; flow rate: 60 mL/min; gradient: 5% B to 45% B in 7 min; 220 nm; Rt: 6 min to afford the title compound (16 mg, 11% yield for two steps) as a white solid.

Formula: C₂₁H₂₈N₂O₆ Exact Mass: 404.19 Molecular Weight: 404.46.

Analytical data: ¹H NMR (300 MHz, Methanol-d₄) δ 8.06 (d, J=6.8 Hz, 1H), 7.63-7.48 (m, 3H), 6.86-6.82 (m, 2H), 5.23 (d, J=6.6 Hz, 1H), 4.23 (t, J=3.3 Hz, 1H), 4.09 (dd, J=6.6 Hz, 2.7 Hz, 1H), 4.05-4.01 (2m, 1H), 3.70-3.58 (m, 4H), 3.13 (s, 6H), 2.50 (s, 3H). ESI-MS [M+H]⁺ calcd for (C₂₁H₂₈N₂O₆H⁺) 405.20, found 405.10.

### EXAMPLE 28

### (2R,3S,4S,5S,6R)-2-((R)-(4-(3-Aminobenzo[d]isoxazol-5-yl)-2-methylphenyl) (hydroxy)methyl)-6-(hydroxymethyl)-tetrahydro-2H-pyran-3,4,5-triol

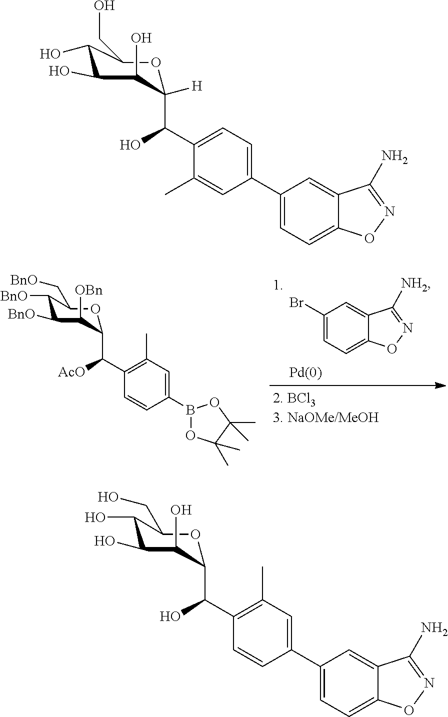

Following Scheme D, Intermediate 106R and commercially purchased 5-bromobenzo[d]isoxazol-3-amine and 2^{nd} Generation Precatalyst-Xphos were reacted via the standard Suzuki coupling procedure (1.5 h at 80° C.), followed first by deprotection protocol B (30 min at −78° C.), then by deprotection protocol A (1 h at rt), and then then by purification using Prep-HPLC with conditions: column Atlantis Prep T3 OBD Column, 19*250 mm 10 um; mobile phase A: Water (0.05% TFA), mobile phase B: CH₃CN; flow rate: 20 mL/min; gradient: 20% B to 44% B in 7 min; 254/220 nm; Rt: 6.32 min to afford the title compound (11 mg, 8% yield for two steps) as a yellow solid.

Formula: C₂₁H₂₄N₂O₇ Exact Mass: 416.16 Molecular Weight: 416.42.

Analytical data: ¹H NMR (300 MHz, Methanol-d₄) δ 8.00 (d, J=1.2 Hz, 1H), 7.81 (2dd, J=8.7 Hz, 1.8 Hz, 1H), 7.61 (2d, J=8.1 Hz, 1H), 7.52-7.43 (m, 3H), 5.23 (d, J=6.6 Hz, 1H), 4.25 (t, J=3.3 Hz, 1H), 4.11 (2dd, J=6.9 Hz, 2.7 Hz, 1H), 4.06-4.02 (m, 1H), 3.71-3.65 (m, 4H), 2.50 (s, 3H). ESI-MS [M+H]⁺ calcd for (C₂₁H₂₄N₂O₇H⁺) 417.17, found 417.3.

### EXAMPLE 29

### 7-(4-((R)-Hydroxy ((2R,3S,4S,5S,6R)-3,4,5-trihydroxy-6-(hydroxymethyl)-tetrahydro-2H-pyran-2-yl)methyl)-3,5-dimethylphenyl)isoquinolin-1 (2H)-one

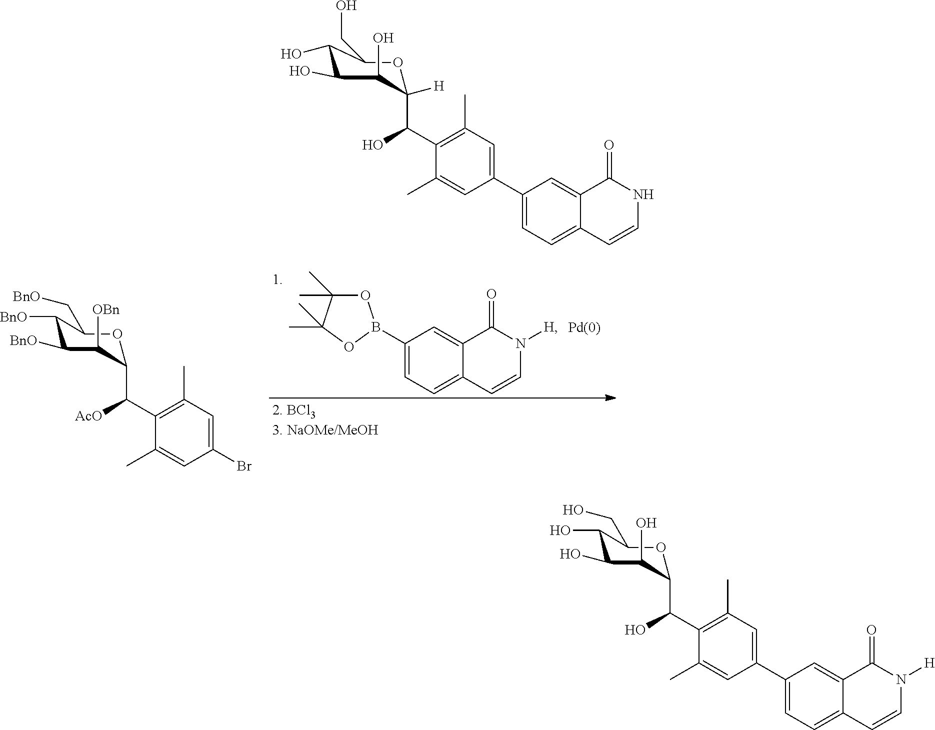

Following Scheme B, Intermediate 115R and commercially available 7-(4,4,5,5-tetramethyl-1,3,2-dioxaborolan-2-yl)isoquinolin-1 (2H)-one were reacted via the standard Suzuki coupling procedure (0.5 h at 80° C.), followed first by deprotection protocol B (30 min at −78° C.), then by deprotection protocol A (1 h at rt), then by purification using Prep-HPLC with conditions: XBridge Prep OBD C18 Column 30×150 mm 5 um; mobile phase A: Water (0.05% TFA), mobile phase B: CH₃CN; flow rate: 20 mL/min; gradient: 5% B to 45% B in 7 min; 254 nm; Rt: 5.68 min to afford the title compound (R isomer, assumed, 26.6 mg, 24% yield for two steps) as a white solid

Formula: C₂₄H₂₇NO₇ Exact Mass: 441.18 Molecular Weight: 441.47.

Analytical data: ¹H NMR (300 MHz, Methanol-d₄) δ 8.54 (s, 1H), 8.04 (d, J=8.1 Hz, 1H), 7.72 (d, J=8.1 Hz, 1H), 7.38 (s, 2H), 7.18 (d, J=6.9 Hz, 1H), 6.71 (2d, J=6.9 Hz, 1H), 5.35 (d, J=8.4 Hz, 1H), 4.44 (d, J=8.4 Hz, 1H), 4.37 (m, 1H), 3.91-3.88 (m, 1H), 3.73 (t, J=9.0 Hz, 1H), 3.62-3.52 (m, 2H), 3.41-3.35 (m, 1H), 2.59 (s, 6H). ESI-MS [M+H]⁺ calcd for (C₂₄H₂₇NO₇H⁺), 442.19, found 442.2.

### EXAMPLE 30

### 7-(4-((S)-hydroxy ((2R,3S,4S,5S,6R)-3,4,5-trihydroxy-6-(hydroxymethyl)tetrahydro-2H-pyran-2-yl)methyl)-3,5-dimethylphenyl)isoquinolin-1 (2H)-one

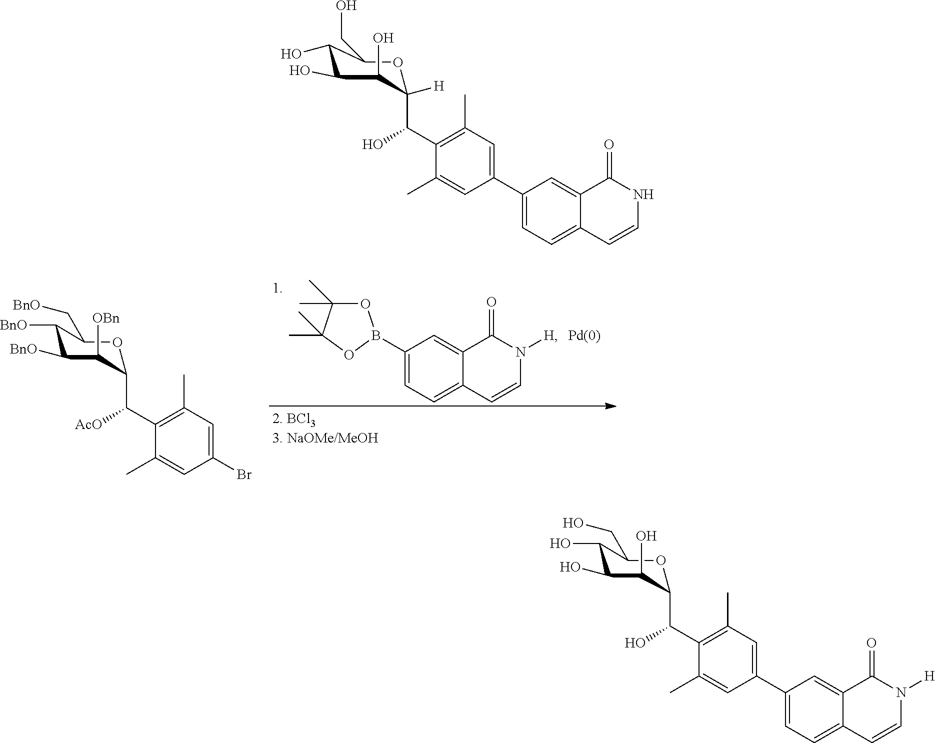

Following Scheme B, Intermediate 115S and commercially available 7-(4,4,5,5-tetramethyl-1,3,2-dioxaborolan-2-yl)isoquinolin-1 (2H)-one were reacted via the standard Suzuki coupling procedure (0.5 h at 80° C.), followed first by deprotection protocol B (30 min at −78° C.), then by deprotection protocol A (1 h at rt), then by purification using Prep-HPLC with conditions: column XSelect CSH Prep C18 OBD Column, 5 um, 19*150 mm; mobile phase A: Water (0.05% TFA), mobile phase B: CH₃CN; flow rate: 20 mL/min; gradient: 5% B to 30% B in 10 min; 254 nm; Rt: 8.82 min to afford the title compound (S isomer assumed, 25 mg, 23% yield for two steps) as a white solid.

Formula: C₂₄H₂₇NO₇ Exact Mass: 441.18 Molecular Weight: 441.47.

Analytical data: ¹H NMR (400 MHz, Methanol-d₄) δ 8.54 (s, 1H), 8.01 (2dt, J=8.0 Hz, 1.6 Hz, 1H), 7.73 (d, J=8.4 Hz, 1H), 7.43 (s, 2H), 7.19 (d, J=7.2 Hz, 1H), 6.72 (d, J=7.2 Hz, 1H), 5.50 (d, J=10.0 Hz, 1H), 4.56 (dd, J=10.0 Hz, 1.6 Hz, 1H), 3.95 (dd, J=11.2 Hz, 1.6 Hz, 1H), 3.78-3.69 (m, 2H), 3.66-3.63 (m, 2H), 3.53-3.47 (m, 1H), 2.61 (2s, 6H). ESI-MS [M+H]⁺ calcd for (C₂₄H₂₇NO₇H⁺), 442.19, found 442.15.

### EXAMPLE 31

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-isopropylisoquinolin-1 (2H)-one

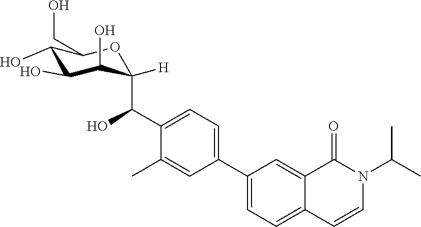

Step 1

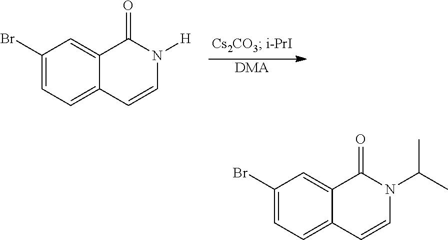

### 7-Bromo-2-isopropylisoquinolin-1 (2H)-one

To a solution of 7-bromoisoquinolin-1 (2H)-one (0.9 g, 4.0 mmol, 1 equiv), 2-iodopropane (0.75 g, 4.4 mmol, 1.1 equiv), Cs₂CO₃ (1.43 g, 4.4 mmol) and DMA (18 mL). The resulting mixture was stirred at 50° C. for 3 hours. Upon completion, the reaction was cooled to rt and poured into ice-water (50 mL). The mixture was extracted with EtOAc (3×50 mL). The combined organic layers were washed with brine (50 mL), dried over anhydrous Na₂SO₄ and filtered. The filtrate was concentrated under vacuum and the residue was purified by silica gel chromatography, eluting with EtOAc in PE (0˜50%) to afford the title compound (0.7 g, 66% yield) as a light yellow solid.

¹H NMR (300 MHz, CDCl₃) δ 8.59 (d, J=2.1 Hz, 1H), 7.70 (dd, J=8.4 Hz, 2.1 Hz, 1H), 7.37 (d, J=8.4 Hz, 1H), 7.16 (d, J=7.5 Hz, 1H), 6.50 (d, J=7.5 Hz, 1H), 5.42-5.33 (m, 1H), 1.39 (d, J=6.9 Hz, 6H). ESI-MS calcd for (C₁₂H₁₂BrNO) [M+H]⁺ 266.01, found 266.0, 268.0.

Step 2

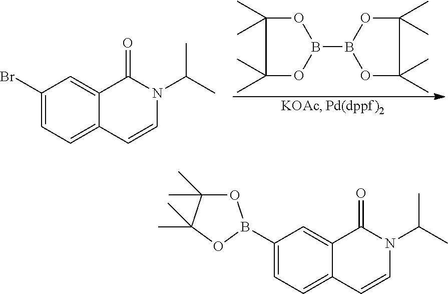

### 2-Isopropyl-7-(4,4,5,5-tetramethyl-1,3,2-dioxaborolan-2-yl)isoquinolin-1 (2H)-one

A solution of the product from the previous step (0.65 g, 2.44 mmol, 1 equiv), bis(pinacolato)diboron (0.68 g, 2.7 mmol, 1.1 equiv), KOAc (0.72 g, 7.32 mmol, 0.1 equiv) and Pd(dppf)Cl₂ (0.2 g, 0.24 mmol, 3 equiv) in dioxane (10 mL) was heated at 80° C. with stirring for 3 h. Upon completion, the reaction was cooled to rt and concentrated under vacuum, and the residue was purified by silica gel chromatography, eluting with EtOAc in PE (0-50%) to afford the title compound (0.75 g, 85% yield) as a light yellow solid.

ESI-MS calcd for (C18H₂₄BNO₃) [M+H]⁺ 314.2, found 314.0.

Step 3

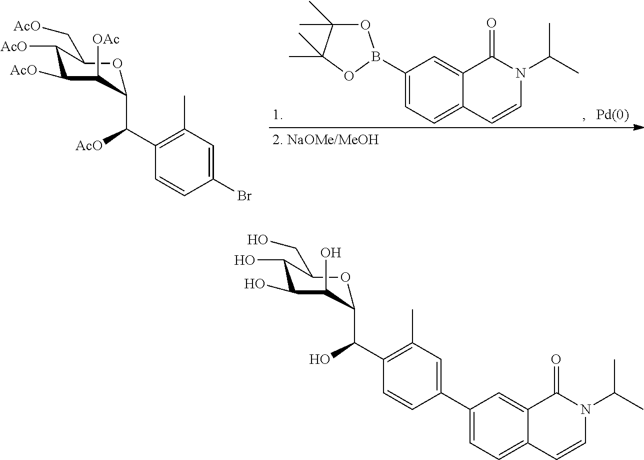

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-isopropylisoquinolin-1 (2H)-one

Following Scheme A, Intermediate 104R and the product from the previous step were reacted via the standard Suzuki coupling procedure (40 min at 80° C.), followed first by deprotection protocol A (2 h at rt), then by purification using Prep-HPLC with conditions: column XBridge Shield Prep C18 OBD Column, 19×150 mm, 5 μm; mobile phase A: water with 0.05% NH₄HCO₃, mobile phase B: CH₃CN; flow rate: 20 mL/min; gradient: 5% B to 32% B in 20 min; 254 nm, Rt: 20.03 min to afford the title compound (33.4 mg, 30% yield for two steps) as a white solid.

Formula: C₂₆H₃₁NO₇ Exact Mass: 469.21 Molecular Weight: 469.53.

Analytical data: ¹H NMR (300 MHz, CD₃OD) δ: 8.58 (d, J=1.5 Hz, 1H), 8.00 (dd, J=8.4 Hz, 2.1 Hz, 1H), 7.71-7.55 (m, 4H), 7.47 (d, J=7.5 Hz, 1H), 6.79 (d, J=7.5 Hz, 1H), 5.42-5.31 (2m, 1H), 5.26 (d, J=6.6 Hz, 1H), 4.26 (t, J=3.0 Hz, 1H), 4.12 (dd, J=6.9 Hz, 2.4 Hz, 1H), 4.08-4.04 (m, 1H), 3.76-3.63 (m, 4H), 2.52 (s, 3H), 1.44 (d, J=6.9 Hz, 6H). ESI-MS [M+H]⁺ calcd for (C₂₆H₃₁NO₇H⁺) 470.22, found 470.15.

### EXAMPLE 32

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-isopropyl-3,4-dihydroisoquinolin-1 (2H)-one

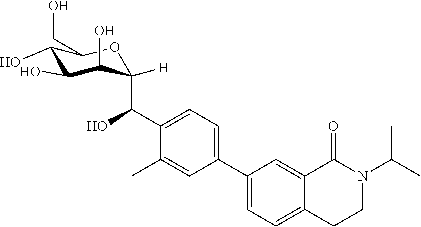

Step 1

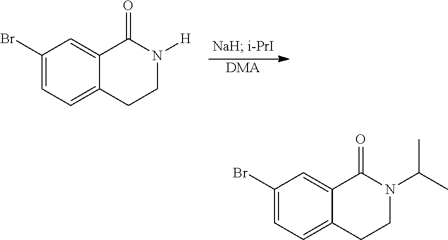

### 7-Bromo-2-isopropyl-3,4-dihydroisoquinolin-1 (2H)-one

To a solution of 7-bromo-3,4-dihydroisoquinolin-1 (2H)-one (1.0 g, 4.45 mmol) in DMA (10 mL) was added NaH (128 mg, 5.34 mmol) under N₂. The reaction was stirred for 1 h, at which time 2-iodopropane (910 mg, 534 mmol) was added. The reaction was stirred for overnight, then quenched by H₂O, and extracted with EtOAc (3×30 mL). The combined organic layers were washed with brine (3×30 mL) and concentrated under reduced pressure. The residue was purified by silica gel chromatography, eluting with EtOAc in PE (0-25%) to give the title compound (560 mg, 46% yield) as a white solid.

¹H NMR (400 MHz, CDCl₃) δ 8.24 (d, J=2.0 Hz, 1H), 7.53 (dd, J=8.0 Hz, 2.0 Hz, 1H), 7.07 (d, J=8.0, 1H), 5.12-5.05 (m, 1H), 3.44 (t, J=6.8 Hz, 2H), 2.91 (2t, J=6.4 Hz, 2H), 1.22 (d, J=6.8 Hz, 6H). ESI-MS calcd for (C₁₂H₁₄BrNO) [M+H]⁺ 268.03, found 268.0, 270.0.

Step 2

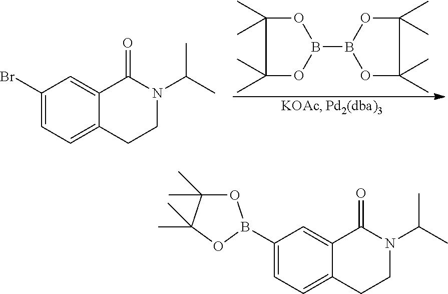

### 2-Isopropyl-7-(4,4,5,5-tetramethyl-1,3,2-dioxaborolan-2-yl)-3,4-dihydroisoquinolin-1 (2H)-one

To a solution of the product from the previous step (430 mg, 1.60 mmol), bis(pinacolato)diboron (445 mg, 1.75 mmol) and KOAc (235 mg, 2.36 mmol) in dioxane (10.0 mL) was added Pd₂(dba)₃ (83 g, 0.08 mmol) under N₂. The resulting mixture was heated to 85° C. for 3 h. After completion, the reaction mixture was concentrated in vacuo. The residue was purified by silica gel chromatography, eluting with EtOAc in PE (0-15%) to give the title compound (120 mg, 34% yield).

ESI-MS Calc'd for (C₁₈H₂₆BNO₃) [M+H]⁺ 316.2, found 316.15.

Step 3

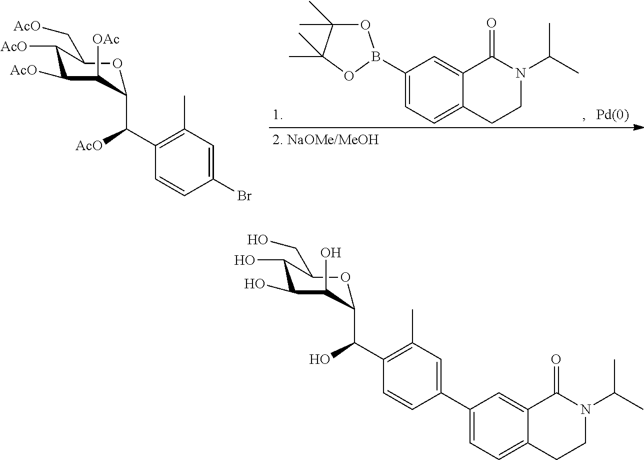

### 7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-isopropyl-3,4-dihydroisoquinolin-1 (2H)-one

Following Scheme A, Intermediate 104R and the product from the previous step were reacted via the standard Suzuki coupling procedure (40 min at 80° C.), followed first by deprotection protocol A (2 h at rt), then by purification using Prep-HPLC with conditions: column XSelect CSH Prep C18 OBD Column, 5 um, 19*150 mm; mobile phase A: Water (0.05% TFA), mobile phase B: CH₃CN; flow rate: 20 mL/min; gradient: 5% B to 15% B in 3 min; 254 nm; Rt: 9.85 min to afford the title compound (16 mg, 13% yield for two steps) as a white solid.

Formula: C₂₆H₃₃NO₇ Exact Mass: 471.23 Molecular Weight: 471.54.

Analytical data: ¹H NMR (300 MHz, Methanol-d₄) δ 8.19 (d, J=2.1 Hz, 1H), 7.73 (dd, J=7.8 Hz, 1.8 Hz, 1H), 7.60 (d, J=8.1 Hz, 1H), 7.51-7.46 (m, 2H), 7.34 (d, J=7.8 Hz, 1H), 5.23 (d, J=6.6 Hz, 1H), 5.05-4.96 (m, 1H), 4.25-4.23 (m, 1H), 4.10 (dd, J=6.6 Hz, 2.7 Hz, 1H), 4.06-4.02 (m, 1H), 3.71-3.65 (m, 4H), 3.54 (t, J=6.6 Hz, 2H), 3.00 (t, J=6.6 Hz, 2H), 2.49 (s, 3H), 1.25 (d, J=6.9 Hz, 6H). ESI-MS [M+H]⁺ calcd for (C₂₆H₃₃NO₇H) 472.23, found 472.4.

### EXAMPLE 33

### 7-(4-((R)-Hydroxy ((2R,3S,4S,5S,6R)-3,4,5-trihydroxy-6-(hydroxymethyl)-tetrahydro-2H-pyran-2-yl)methyl)-3-(trifluoromethyl)phenyl)-3,4-dihydroisoquinolin-1 (2H)-one

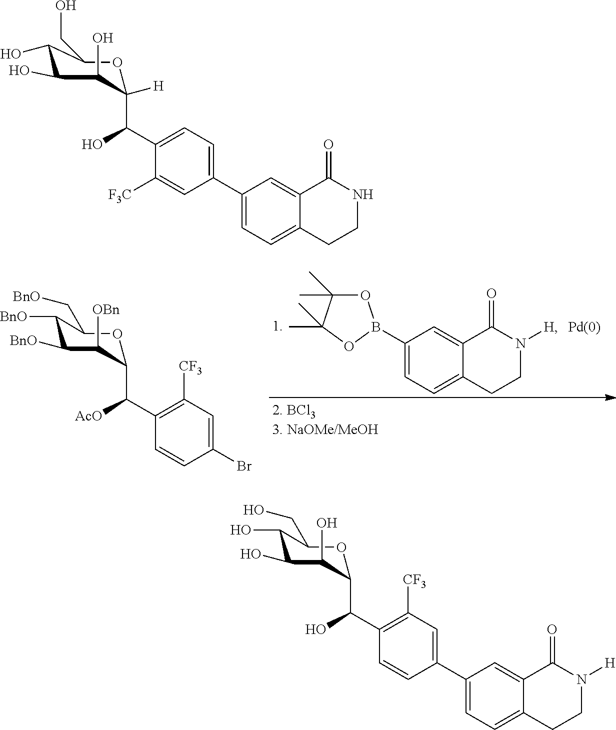

Following Scheme B, Intermediate 109R and commercially available 7-(4,4,5,5-tetramethyl-1,3,2-dioxaborolan-2-yl)-3,4-dihydroisoquinolin-1 (2H)-one were reacted via the standard Suzuki coupling procedure (30 min at 80° C.), followed first by deprotection protocol B (BCl₃, 30 min at −78° C.) then by deprotection protocol A (1 h at rt), then by purification using Prep-HPLC with conditions: column Atlantis Prep T3 OBD C18 Column 19×250 mm 10 um; mobile phase A: Water (0.05% TFA), mobile phase B: CH₃CN; flow rate: 20 mL/min; gradient: 20% B to 38.8% B in 8.5 min; 254 nm/220 nm; Rt: 7.73 min to give the title compound (110 mg, 50% yield for two steps) as a white solid.

Formula: C₂₃H₂₄F₃NO₇ Exact Mass: 483.15 Molecular Weight: 483.43.

Analytical data: ¹H NMR (300 MHz, methanol-d₄) δ ppm 8.24 (s, 1H), 7.96-7.94 (m, 3H), 7.83 (dd, J₁=8.1 Hz, J₂=2.1 Hz, 1H), 7.44 (d, J=8.1 Hz, 1H), 5.36 (d, J=6.9 Hz, 1H), 4.31-4.29 (m, 1H), 4.18 (dd, J=6.9 Hz, J₂=1.2 Hz, 1H), 4.01 (2dd, J=8.7 Hz, J₂=3.3 Hz, 1H), 3.73 (t, J=8.7 Hz, 1H), 3.67-3.59 (m, 3H), 3.55 (t, J=6.9 Hz, 2H), 3.05 (t, J=6.6 Hz, 2H). ESI-MS [M+Na]⁺ calcd for (C₂₃H₂₄F₃NO₇N⁺) 506.14, found 507.15.

### EXAMPLE 34

### 7-(4-((S)-hydroxy ((2R,3S,4S,5S,6R)-3,4,5-trihydroxy-6-(hydroxymethyl)tetrahydro-2H-pyran-2-yl)methyl)-3-(trifluoromethyl)phenyl)-3,4-dihydroisoquinolin-1 (2H)-one

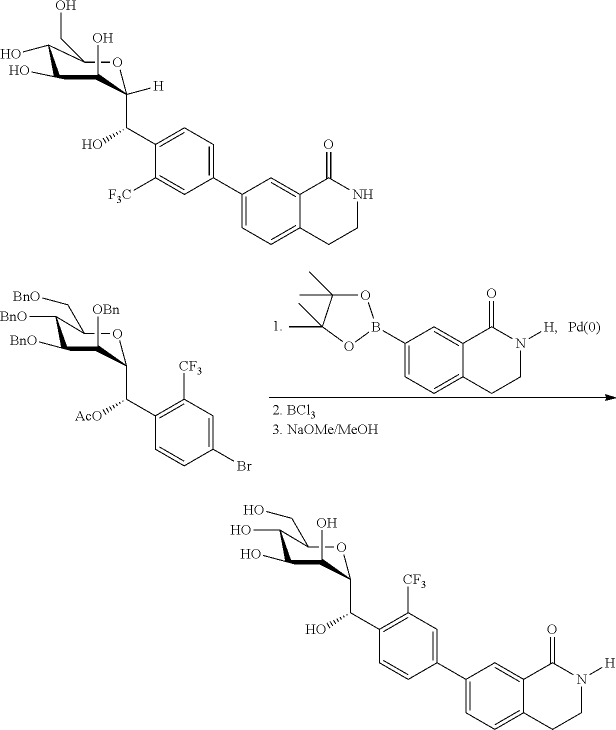

Following Scheme B, Intermediate 109S and commercially available 7-(4,4,5,5-tetramethyl-1,3,2-dioxaborolan-2-yl)-3,4-dihydroisoquinolin-1 (2H)-one were reacted via the standard Suzuki coupling procedure (30 min at 80° C.), followed first by deprotection protocol B (BCl₃, 30 min at −78° C.) then by deprotection protocol A (1 h at rt), then by purification using Prep-HPLC with conditions: column XSelect CSH Prep C18 OBD Column, 5 um, 19*150 mm; mobile phase A: Water (0.05% TFA), mobile phase B: CH₃CN; flow rate: 20 mL/min; gradient: 30% B to 37.1% B in 3.5 min; 254/220 nm; Rt: 3.13 min to give the title compound (80 mg, 42% yield for two steps) as a white solid.

Formula: C₂₃H₂₄F₃NO₇ Exact Mass: 483.15 Molecular Weight: 483.43.

Analytical data: ¹H NMR (300 MHz, methanol-d₄) δ ppm 8.24 (d, J=1.8 Hz, 1H), 8.01-7.95 (m, 3H), 7.83 (dd, J₁=7.8 Hz, J₂=2.1 Hz, 1H), 7.45 (d, J=7.8 Hz, 1H), 5.36 (d, J₁=6.6 Hz, 1H), 4.07 (dd, J₁=6.6 Hz, J₂=3.0 Hz, 1H), 3.90-3.83 (m, 3H), 3.75-3.64 (m, 3H), 3.55 (t, J₁=6.9 Hz, 2H), 3.05 (t, J=6.6 Hz, 2H). ESI-MS [M+H]⁺ calcd for (C₂₃H₂₄F₃NO₇H⁺) 484.16, found 484.15.

### EXAMPLE 35

### 5-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-N-methylnicotinamide

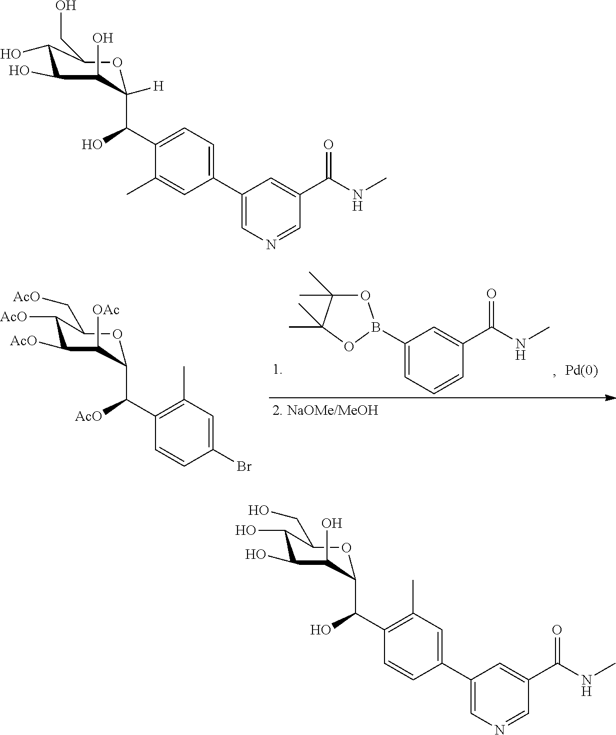

Following Scheme A, Intermediate 104R and commercially available N-methyl-5-(4,4,5,5-tetramethyl-1,3,2-dioxaborolan-2-yl)nicotinamide were reacted via the standard Suzuki coupling procedure (1.5 h at 80° C.), followed first by deprotection protocol A (1 h at rt), then by purification using Prep-HPLC with conditions: column XSelect CSH Prep C18 OBD Column, 5 um, 19*150 mm; mobile phase A: Water (0.05% TFA), mobile phase B: CH₃CN; flow rate: 20 mL/min; gradient: 10% B to 60% B in 9 min; 254/220 nm; Rt: 8.65 min to afford the title compound (40 mg, 34% yield for two steps) as a white solid.

Formula: C₂₁H₂₆N₂O₇ Exact Mass: 418.17 Molecular Weight: 418.44.

Analytical data: ¹H NMR (400 MHz, Methanol-d₄) δ 9.05 (s, 1H), 8.99 (s, 1H), 8.71-8.69 (m, 1H), 7.70 (d, J=8.0 Hz, 1H), 7.63-7.59 (m, 2H), 5.25 (d, J=6.8 Hz, 1H), 4.22 (t, J=3.2 Hz, 1H), 4.10 (dd, J=6.8 Hz, 2.4 Hz, 1H), 4.04-4.01 (2m, 1H), 3.70-3.65 (m, 4H), 2.98 (s, 3H), 2.53 (s, 3H). ESI-MS [M+H]⁺ calcd for (C₂₁H₂₆N₂O₇H⁺) 419.18, found 419.4.

### EXAMPLE 36

### (2R,3S,4S,5S,6R)-2-((R)-Hydroxy (4-(imidazo[1,2-a]pyridin-2-yl)-2-methylphenyl)methyl)-6-(hydroxymethyl)-tetrahydro-2H-pyran-3,4,5-triol

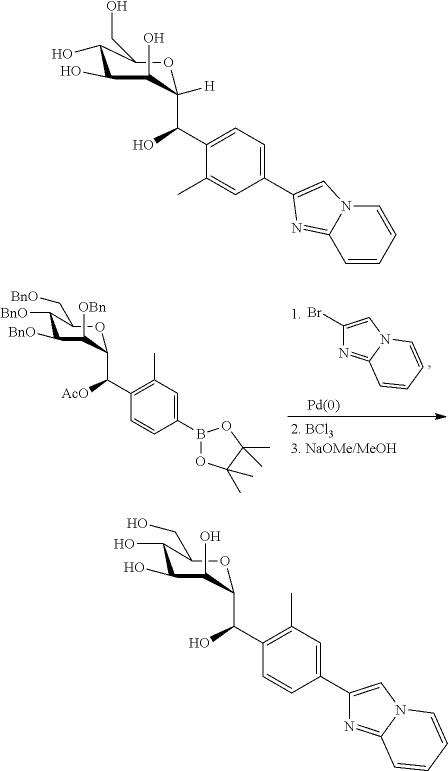

Following Scheme D, Intermediate 106R and commercially available 2-bromo-imidazo[1,2-α]pyridine were reacted via the standard Suzuki coupling procedure (1.5 h at 80° C.), followed first by deprotection protocol B (BCl₃, 30 min at −78° C.), then by deprotection protocol A (1 h at rt), then by purification using Prep-HPLC with conditions: column XBridge Shield RP18 OBD Column, 5 um, 19*150 mm; mobile phase A: Water (10 mmol/L NH₄HCO₃), mobile phase B: CH₃CN; flow rate: 20 mL/min; gradient: 5% B to 55% B in 7 min; 254 nm; Rt: 5.5 min to afford the title compound (30.0 mg, 17% yield for two steps) as a white solid.

Formula: C₂₁H₂₄N₂O₆ Exact Mass: 400.16 Molecular Weight: 400.43.

Analytical data: 1H NMR (400 MHz, Methanol-d₄) δ 8.41 (2dt, J=6.8 Hz, 1.2 Hz, 1H), 8.18 (s, 1H), 7.78-7.75 (m, 2H), 7.59 (d, J=8.0 Hz, 1H), 7.54 (d, J=9.2 Hz, 1H), 7.30 (ddd, J=9.2 Hz, 6.8 Hz, 1.2 Hz, 1H), 6.91 (2td, J=6.8 Hz, 1.2 Hz, 1H), 5.23 (d, J=6.8 Hz, 1H), 4.25 (t, J=3.2 Hz, 1H), 4.11 (2dd, J=6.8 Hz, 2.8 Hz, 1H), 4.06-4.03 (m, 1H), 3.71-3.65 (m, 4H), 2.50 (s, 3H). ESI-MS [M+H]⁺ calcd for (C₂₁H₂₄N₂O₆H⁺) 401.17, found 401.05.

### EXAMPLE 37

### 4′-((R)-Hydroxy ((2R,3S,4S,5S,6R)-3,4,5-trihydroxy-6-(hydroxymethyl)-tetrahydro-2H-pyran-2-yl)methyl)-5′-methyl-N-(pyridin-2-yl)biphenyl-3-carboxamide

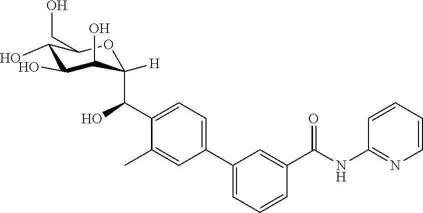

Step 1

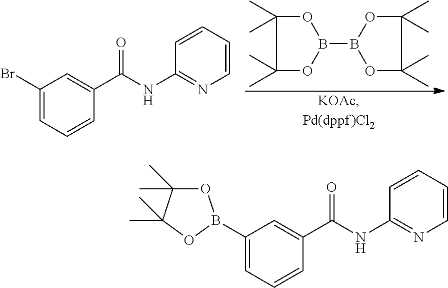

### N-(Pyridin-2-yl)-3-(4,4,5,5-tetramethyl-1,3,2-dioxaborolan-2-yl)benzamide

To a solution of 3-bromo-N-(pyridin-2-yl)benzamide (200 mg, 0.7273 mmol, Bioorg. Med. Chem. Lett. 2005, 15, 1197) in dioxane (3.0 mL) were added bis(pinacolato)diboron (214 mg, 2.1819 mmol), KOAc (214 mg, 2.1819 mmol) and Pd(dppf)Cl₂ (60 mg, 0.073 mmol) under N₂. The resulting mixture was heated to 85° C. for 3 h. After completion, the reaction mixture was cooled to rt and was concentrated under vacuo. The residue was purified by silica gel chromatography, eluting with EtOAc in PE (0-60%) to give the title compound (235 mg, 93% yield).

ESI-MS calcd for (C₁₈H₂₁BN₂O₃) [M+H]⁺ 325.16, found 325.05.

Step 2

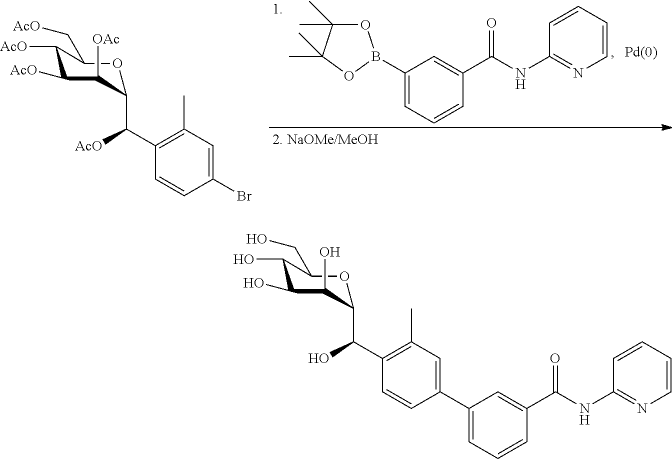

### 4′-((R)-Hydroxy ((2R,3S,4S,5S,6R)-3,4,5-trihydroxy-6-(hydroxymethyl)-tetrahydro-2H-pyran-2-yl)methyl)-5′-methyl-N-(pyridin-2-yl)biphenyl-3-carboxamide

Following Scheme A, Intermediate 104R and the product from the previous step were reacted via the standard Suzuki coupling procedure (40 min at 80° C.), followed first by deprotection protocol A (2 h at rt), then by purification using Prep-HPLC with conditions: column XBridge C18 OBD Prep Column 100 Å, 10 μm, 19 mm×250 mm; mobile phase A: Water (0.05% TFA, mobile phase B: CH₃CN; flow rate: 20 mL/min; gradient: 5% B to 30% B in 15 min; 254/220 nm; Rt: 12.35 min to afford the title compound (45 mg, 34% yield) as a white solid.

Formula: C₂₆H₂₈N₂O₇ Exact Mass: 480.19 Molecular Weight: 480.50.

Analytical data: ¹H NMR (400 MHz, Methanol-d₄) δ 8.36-8.35 (m, 1H), 8.26-8.22 (m, 2H), 7.94-7.82 (m, 3H), 7.65-7.53 (m, 4H), 7.19-7.15 (m, 1H), 5.25 (d, J=6.8 Hz, 1H), 4.25 (t, J=3.2 Hz, 1H), 4.11 (2dd, J=6.8 Hz, 2.8 Hz, 1H), 4.07-4.03 (m, 1H), 3.74-3.62 (m, 4H), 2.51 (2s, 3H). ESI-MS [M+H]⁺ calcd for (C₂₆H₂₈N₂O₇H⁺) 481.2, found 481.4.

### EXAMPLE 38

### 4′-((R)-Hydroxy ((2R,3S,4S,5S,6R)-3,4,5-trihydroxy-6-(hydroxymethyl)-tetrahydro-2H-pyran-2-yl)methyl)-N³,N⁵,3′-trimethylbiphenyl-3,5-dicarboxamide

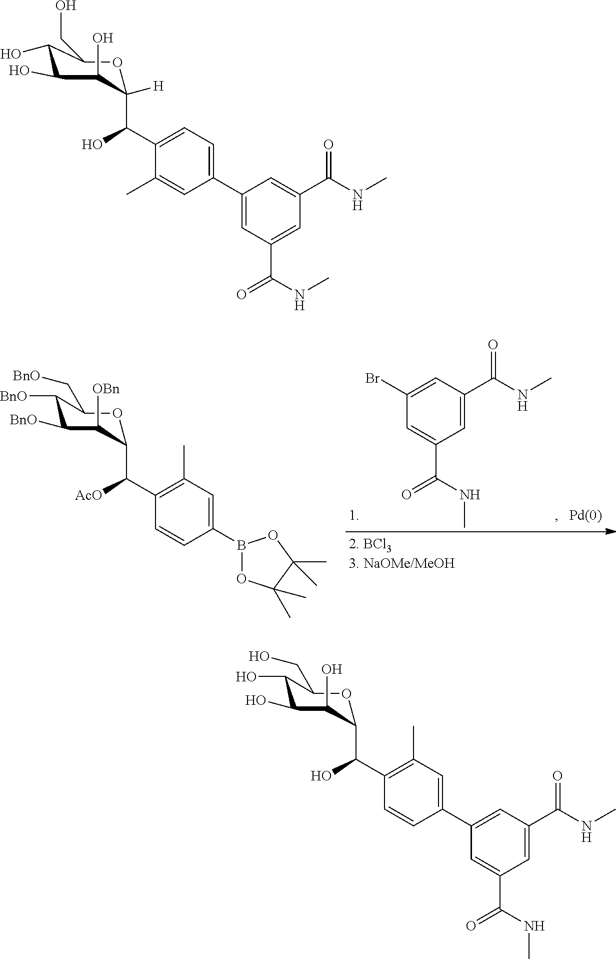

Following Scheme D, Intermediate 106R and 5-bromo-N¹,N³-dimethyliso-phthalamide (J. Med. Chem. 2012, 55, 3945) were reacted via the standard Suzuki coupling procedure (40 min at 80° C.), followed first by deprotection protocol B (BCl₃, 30 min at −78° C.), then by deprotection protocol A (2 h at rt), then by purification using Prep-HPLC with conditions: column XBridge Shield RP18 OBD Column, 5 um, 19*150 mm; mobile phase A: Water (10 mmol/L NH₄HCO₃), mobile phase B: CH₃CN; flow rate: 20 mL/min; gradient: 25% B to 75% B in 7 min; 254 nm; Rt: 6.32 min to afford the title compound (34 mg, 42% yield for two steps) as a white solid.

Formula: C₂₄H₃₀N₂O₈ Exact Mass: 474.20 Molecular Weight: 474.50.

Analytical data: ¹H NMR (400 MHz, Methanol-d₄) δ 8.22-8.21 (2m, 3H), 7.68-7.52 (m, 3H), 5.24 (d, J=6.8 Hz, 1H), 4.24 (t, J=3.2 Hz, 1H), 4.10 (dd, J=6.8 Hz, 2.4 Hz, 1H), 4.05-4.02 (m, 1H), 3.75-3.59 (m, 4H), 2.96 (s, 6H), 2.52 (s, 3H). ESI-MS [M+H]⁺ calcd for (C₂₄H₃₀N₂O₈NH₄⁺) 475.21, found 475.05.

### EXAMPLE 39

### 4′-((R)-Hydroxy ((2R,3S,4S,5S,6R)-3,4,5-trihydroxy-6-(hydroxymethyl)-tetrahydro-2H-pyran-2-yl)methyl)-N-methyl-5′-(trifluoromethyl)biphenyl-3-carboxamide

Step 1

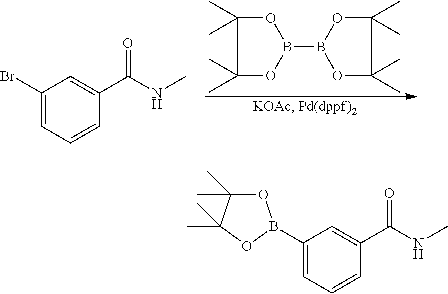

### N-Methyl-3-(4,4,5,5-tetramethyl-1,3,2-dioxaborolan-2-yl)benzamide

To a solution of commercially available 3-bromo-N-methylbenzamide (2.0 g, 9.35 mmol) in dioxane (20 mL) were added bis(pinacolato)diboron (2.65 g, 10.28 mmol), KOAc (2.75 g, 28.05 mmol) and Pd (dppf)Cl₂ (763 mg, 0.935 mmol) at rt under N₂ atmosphere. The resulting mixture was stirred for 1 h at 80° C. After completion, the reaction was cooled to rt. Water (2 mL) was added to the reaction. The resulting mixture was extracted with EtOAc (3×5 mL). The combined organic layers were washed with brine (5 mL) and dried over anhydrous Na₂SO₄. The filtrate was concentrated under reduced pressure. The residue was purified by silica gel chromatography, eluting with EtOAc in PE (0-20%) to afford the title compound (2.35 g, 96% yield) as a white solid.

ESI-MS calcd for (C₁₀H₈BrNO) [M+Na]⁺284.1, found 284.14.

Step 2

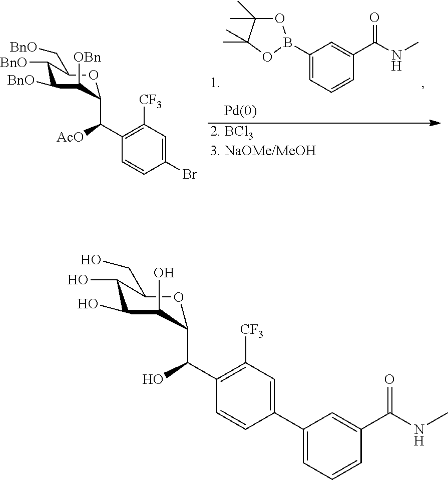

### 4′-((R)-Hydroxy ((2R,3S,4S,5S,6R)-3,4,5-trihydroxy-6-(hydroxymethyl)-tetrahydro-2H-pyran-2-yl)methyl)-N-methyl-5′-(trifluoromethyl)biphenyl-3-carboxamide

Following Scheme B, Intermediate 109R and the product from the previous step were reacted via the standard Suzuki coupling procedure (30 min at 80° C.), followed first by deprotection protocol B (BCl₃, 30 min at −78° C.), then by deprotection protocol A (1 h at rt), then by purification using Prep-HPLC with conditions: Xbridge Shield RP C18 OBD Column 19×150 mm 5 um; mobile phase A: Water (0.05% TFA), mobile phase B: CH₃CN; flow rate: 20 mL/min; gradient: 5% B to 45% B in 7 min; 254 nm; Rt: 5.8 min) to give the title compound (110 mg, 57% yield for two steps) as a white solid.

Formula: C₂₂H₂₄F₃NO₇ Exact Mass: 471.15 Molecular Weight: 471.42.

Analytical data: ¹H NMR (300 MHz, methanol-d₄) δ ppm 8.12 (t, J=1.5 Hz, 1H), 7.97 (s, 3H), 7.84 (dd, J₁=7.8 Hz, J₂=1.2 Hz, 2H), 7.58 (t, J=7.8 Hz, 1H), 5.37 (d, J₁=6.9 Hz, 1H), 4.32-4.30 (m, 1H), 4.18 (dd, J=6.9 Hz, J₂=1.5 Hz, 1H), 4.01 (2dd, J₁=8.7 Hz, J₂=3.3 Hz, 1H), 3.73 (t, J=8.4 Hz, 1H), 3.66-3.57 (m, 3H), 2.95 (s, 3H). ESI-MS [M+Na]⁺ calcd for (C₂₂H₂₄F₃NO₇Na⁺) 494.14, found 494.05.

### EXAMPLE 40

### 4′-((S)-hydroxy ((2R,3S,4S,5S,6R)-3,4,5-trihydroxy-6-(hydroxymethyl)tetrahydro-2H-pyran-2-yl)methyl)-N-methyl-3′-(trifluoromethyl)-[1,1′-biphenyl]-3-carboxamide

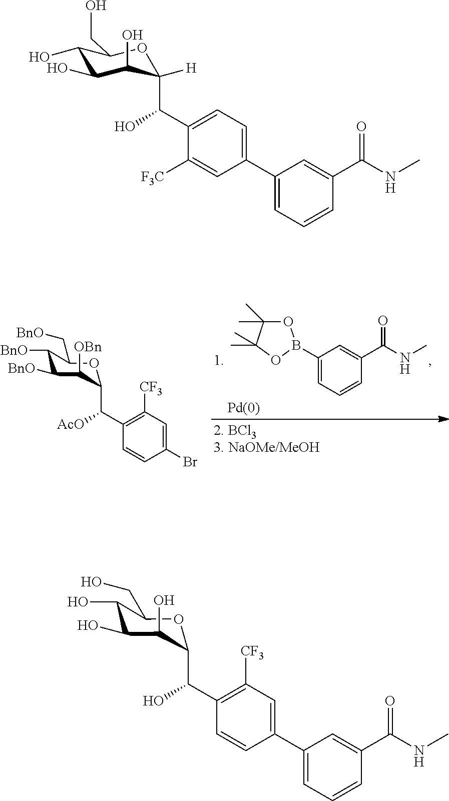

Following Scheme B, Intermediate 109S and N-methyl-3-(4,4,5,5-tetramethyl-1,3,2-dioxaborolan-2-yl)benzamide were reacted via the standard Suzuki coupling procedure (30 min at 80° C.), followed first by deprotection protocol B (BCl₃, 30 min at −78° C.), then by deprotection protocol A (1 h at rt), then by purification using Prep-HPLC with conditions: column: Atlantis Prep T3 OBD Column, 19*250 mm 10 um; mobile phase A: Water (0.05% TFA), mobile phase B: CH₃CN; flow rate: 20 mL/min; gradient: 15% B to 45% B in 8 min; 254/220 nm; Rt: 5.92 min to give the title compound (S isomer assumed, 98 mg, 54% yield for two steps) as a white solid.

Formula: C₂₂H₂₄F₃NO₇ Exact Mass: 471.15 Molecular Weight: 471.42.

Analytical data: ¹H NMR (300 MHz, methanol-d₄) δ ppm 8.12 (t, J=1.5 Hz, 1H), 7.99 (s, 3H), 7.85 (dd, J₁=7.8 Hz, J₂=1.8 Hz, 2H), 7.59 (t, J=7.5 Hz, 1H), 5.37 (d, J₁=6.6 Hz, 1H), 4.07 (dd, J₁=6.3 Hz, J₂=3.3 Hz, 1H), 3.91-3.83 (m, 3H), 3.76-3.65 (m, 3H), 2.95 (s, 3H). ESI-MS [M+Na]⁺ calcd for (C₂₂H₂₄F₃NO₇Na⁺⁾ 494.15, found 494.10.

### EXAMPLE 41

### 4′-((R)-Hydroxy ((2R,3S,4S,5S,6R)-3,4,5-trihydroxy-6-(hydroxymethyl)-tetrahydro-2H-pyran-2-yl)methyl)-N,N,5′-trimethylbiphenyl-3-carboxamide

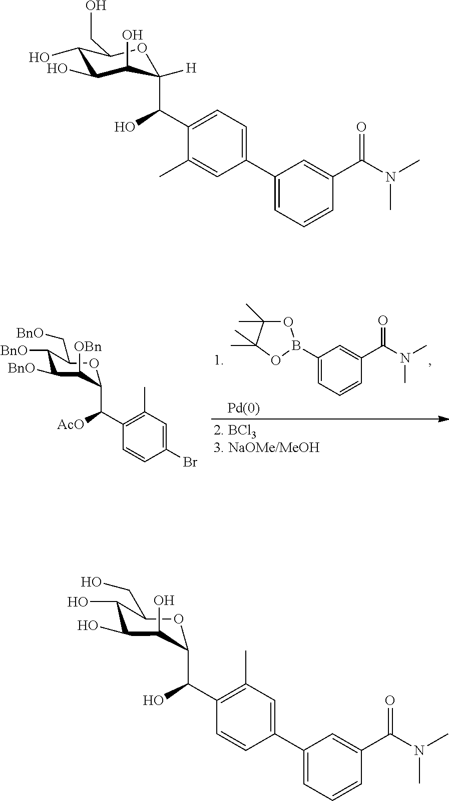

Following Scheme B, Intermediate 103R and N,N-dimethyl-3-(4,4,5,5-tetramethyl-1,3,2-dioxaborolan-2-yl)benzamide (Eur. J. Med. Chem. 2015, 96, 382) were reacted via the standard Suzuki coupling procedure (3 h at 80° C.), followed first by deprotection protocol B (BCl₃, 30 min at −78° C.), then by deprotection protocol A (1 h at rt), then by purification using Prep-HPLC with conditions: column XBridge Prep OBD C18 Column, 30×150 mm 5 um; mobile phase A: Water (10 mmol/L NH₄HCO₃), mobile phase B: CH₃CN; flow rate: 60 mL/min; gradient: 3% B to 40% B in 7 min; 220 nm; RT1: 5.32 min to afford the title compound (35 mg, 34% yield) as a white solid.

Formula: C₂₃H₂₉NO₇ Exact Mass: 431.19 Molecular Weight: 431.48.

Analytical data: ¹H NMR (300 MHz, Methanol-d₄) δ 7.74-7.70 (m, 1H), 7.65-7.60 (m, 2H), 7.54-7.45 (m, 3H), 7.39-7.36 (m, 1H), 5.23 (d, J=6.9 Hz, 1H), 4.24 (t, J=3.3 Hz, 1H), 4.09 (dd, J=6.9 Hz, 2.4 Hz, 1H), 4.05-4.02 (m, 1H), 3.70-3.64 (m, 4H), 3.13 (s, 3H), 3.04 (s, 3H), 2.49 (s, 3H). ESI-MS [M+H]⁺ calcd for (C₂₃H₂₉NO₇H⁺) 432.20, found 432.05.

### EXAMPLE 42

### 5-Cyano-4′-((R)-hydroxy ((2R,3S,4S,5S,6R)-3,4,5-trihydroxy-6-(hydroxymethyl)-tetrahydro-2H-pyran-2-yl)methyl)-N,5′-dimethylbiphenyl-3-carboxamide

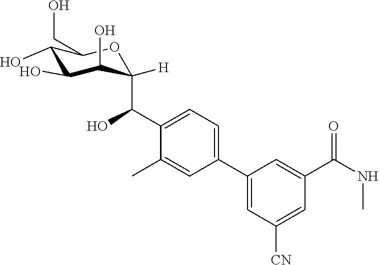

Step 1

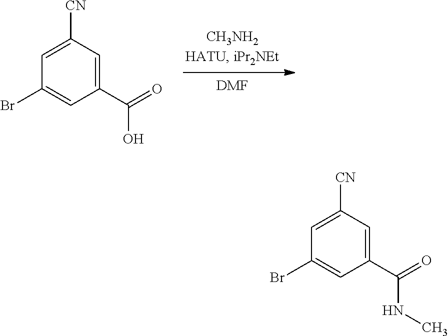

### 3-Bromo-5-cyano-N-methylbenzamide

To a solution of 3-bromo-5-cyanobenzoic acid (500 mg, 2.2 mmol) in DMF (5 mL) were added HATU (1.67 g, 4.4 mmol) and iPr₂NEt (851 mg, 6.6 mmol). The mixture was stirred for 15 min at rt. MeNH₂ (2M solution in THF, 5 mL, 10 mmol) was then added dropwise and the resulting mixture was stirred for 2 h at rt. After completion, water (20 mL) was added to the reaction. The resulting mixture was extracted with EtOAc (3×50 mL). The combined organic layers were washed with brine (50 mL), dried over anhydrous Na₂SO₄ and filtered. The filtrate was concentrated under reduced pressure. The residue was purified by silica gel chromatography, eluting with EtOAc in PE (0-70%) to afford the title compound (450 mg, 86% yield) as a yellow solid.

MS (ESI+) [M+H]⁺ calcd for (C₉H₇BrN₂OH⁺⁾ 238.98, found 238.85, 240.85.

Step 2

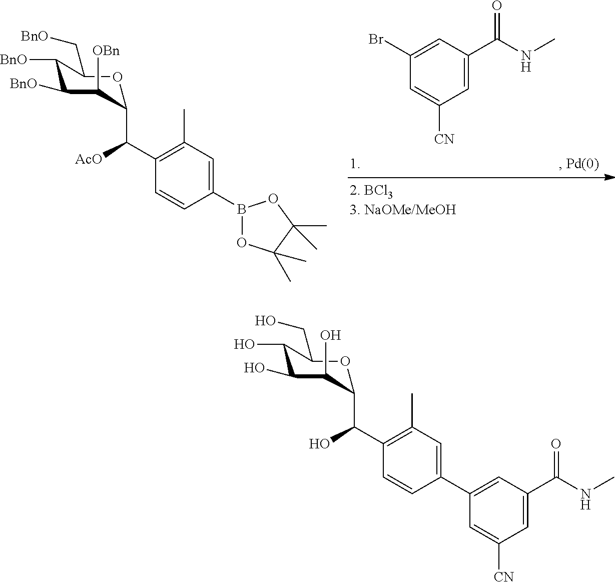

### 5-Cyano-4′-((R)-hydroxy ((2R,3S,4S,5S,6R)-3,4,5-trihydroxy-6-(hydroxymethyl)-tetrahydro-2H-pyran-2-yl)methyl)-N,5′-dimethylbiphenyl-3-carboxamide

Following Scheme D, Intermediate 106R and 3-bromo-5-cyano-N-methylbenzamide were reacted via the standard Suzuki coupling procedure (3 h at 80° C.), followed first by deprotection protocol B (BCl₃, 30 min at −78° C.) then by deprotection protocol A (1 h at rt), then by purification using Prep-HPLC with conditions: column XBridge Prep OBD C18 Column 19×150 mm 5 um C-0013; mobile phase A: Water (10 mmol/L NH₄HCO₃), mobile phase B: CH₃CN; flow rate: 20 mL/min; gradient: 3% B to 27% B in 11 min; 254 nm; Rt: 9.83 min to afford the title compound (50 mg, 48% yield for two steps) as a white solid.

Formula: C₂₃H₂₆N₂O₇ Exact Mass: 442.17 Molecular Weight: 442.46.

Analytical data: ¹H NMR (400 MHz, Methanol-d₄) δ 8.35 (t, J=1.2 Hz, 1H), 8.14 (t, J=1.2 Hz, 1H), 8.10 (t, J=1.2 Hz, 1H), 7.66 (d, J=8.0 Hz, 1H), 7.57 (dd, J=8.0 Hz, 1.6 Hz, 1H), 7.54 (s, 1H), 5.24 (d, J=6.8 Hz, 1H), 4.23 (t, J=2.8 Hz, 1H), 4.10 (dd, J=6.8 Hz, 2.8 Hz, 1H), 4.06-4.01 (2m, 1H), 3.70-3.62 (m, 4H), 2.96 (s, 3H), 2.52 (s, 3H). ESI-MS [M+Na]⁺ calcd for (C₂₃H₂₆N₂O₇Na⁺⁾ 465.16, found 465.10.

### EXAMPLE 43

### 4-Cyano-4′-((R)-hydroxy ((2R,3S,4S,5S,6R)-3,4,5-trihydroxy-6-(hydroxymethyl)-tetrahydro-2H-pyran-2-yl)methyl)-N,5′-dimethylbiphenyl-3-carboxamide

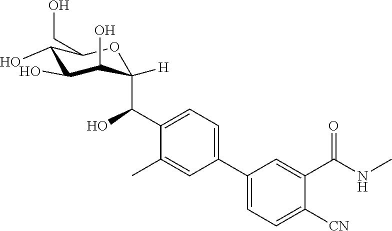

Step 1

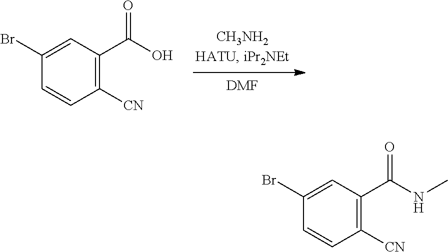

### 5-Bromo-2-cyano-N-methylbenzamide

To a solution of 5-bromo-2-cyanobenzoic acid (500 mg, 2.2 mmol) in DMF (5 mL) were added HATU (1.672 g, 4.4 mmol) and iPr₂NEt (851 mg, 6.6 mmol). The mixture was stirred for 15 min at rt. MeNH₂ (2M solution in THF, 5 ml, 10 mmol) was then added dropwise and the resulting mixture was stirred for 2 h at rt. After completion, water (20 mL) was added to the reaction. The resulting mixture was extracted with EtOAc (3×50 mL). The combined organic layers were washed with brine (50 mL) and dried over anhydrous Na₂SO₄. The filtrate was concentrated under reduced pressure. The residue was purified by silica gel chromatography, eluting with EtOAc in PE (0-70%) to afford the title compound (400 mg, 76% yield) as a light yellow solid. MS (ESI+) [M+H]⁺ calcd for (C₉H₇BrN₂OH⁺⁾ 238.98, found 239.05, 241.05.

Step 2

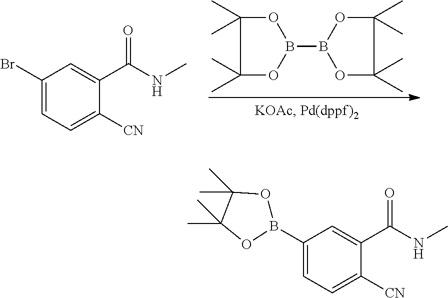

### 2-Cyano-N-methyl-5-(4,4,5,5-tetramethyl-1,3,2-dioxaborolan-2-yl)benzamide

To a solution of the product from the previous step (200 mg, 0.84 mmol) in dioxane (5 mL) were added bis(pinacolato)diboron (234 mg, 0.92 mmol), KOAc (247 mg, 2.52 mmol) and Pd(dppf)Cl₂ (68 mg, 0.084 mmol) at rt under N₂ atmosphere. The resulting mixture was stirred for 1 h at 80° C. After completion, the reaction was cooled to rt. Water (2 mL) was added to the reaction. The resulting mixture was extracted with EtOAc (3×5 mL). The combined organic layers were washed with brine (5 mL) and dried over anhydrous Na₂SO₄. The filtrate was concentrated under reduced pressure. The residue was purified by silica gel chromatography, eluting with EtOAc in PE (0-75%) to afford the title compound (120 mg, 50% yield) as an off-white solid.

MS (ESI+) [M+H]⁺ calcd for (C₁₅H₁₉BN₂O₃H⁺) 287.14, found 287.05.

Step 3

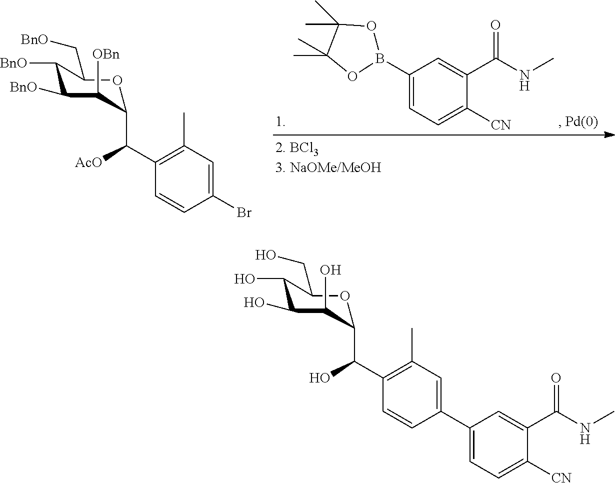

### 4-Cyano-4′-((R)-hydroxy ((2R,3S,4S,5S,6R)-3,4,5-trihydroxy-6-(hydroxymethyl)-tetrahydro-2H-pyran-2-yl)methyl)-N,5′-dimethylbiphenyl-3-carboxamide

Following Scheme B, Intermediate 103R and the product from the previous step were reacted via the standard Suzuki coupling procedure (3 h at 80° C.), followed first by deprotection protocol B (BCl₃, 30 min at −78° C.), then by deprotection protocol A (2 h at rt), then by purification using Prep-HPLC with conditions: column XBridge Prep OBD C18 Column 19×150 mm 5 um C-0013; mobile phase A: Water (10 mmol/L NH₄HCO₃), mobile phase B: CH₃CN; flow rate: 20 mL/min; gradient: 10% B to 30% B in 7 min; 254 nm; Rt: 5.42 min to afford the title compound (18.0 mg, 20% yield for two steps) as a white solid.

Formula: C₂₃H₂₆N₂O₇ Exact Mass: 442.17 Molecular Weight: 442.46.

Analytical data: ¹H NMR (400 MHz, Methanol-d₄) δ 8.05-7.97 (m, 3H), 7.66 (d, J=8.1 Hz, 1H), 7.57-7.51 (2m, 2H), 5.25 (d, J=6.6 Hz, 1H), 4.24 (t, J=3.0 Hz, 1H), 4.10 (dd, J=6.6, 3.0 Hz, 1H), 4.06-4.02 (m, 1H), 3.72-3.65 (m, 4H), 3.26 (s, 3H), 2.51 (2s, 3H). ESI-MS [M+H]⁺ calcd for (C₂₃H₂₆N₂O₇H⁺) 443.18, found 443.3.

### EXAMPLE 44

### 4′-((R)-azido((2R,3S,4R,5S,6R)-3,4,5-trihydroxy-6-(hydroxymethyl)tetrahydro-2H-pyran-2-yl)methyl)-N,3′-dimethylbiphenyl-3-carboxamide

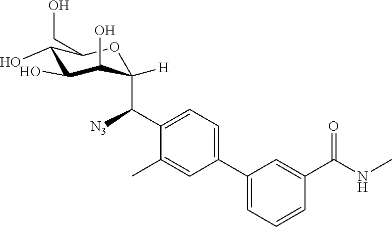

Following Scheme B, Intermediate 11 OR and commercially available N-methyl-3-(4,4,5,5-tetramethyl-[1,3,2]dioxaborolan-2-yl)-benzamide were reacted via the standard Suzuki coupling procedure (2 h at 80° C.) followed by benzyl deprotection protocol B (2 h at −78° C.). The resulting residue was purified by HPLC (C18, 15*150 mm column; eluent: acetonitrile/water (0.05% TFA) to give the title compound in 24% yield.

Formula: C₂₂H₂₆N₄O₆ Exact Mass: 442.19 Molecular Weight: 442.47

¹H NMR (400 MHz, methanol-d₄) δ ppm 8.09 (d, J=3.9 Hz, 1H), 7.80 (d, J=4.7 Hz, 2H), 7.46-7.66 (m, 4H), 5.19 (dd, J=7.8, 5.5 Hz, 1H), 4.20-4.29 (m, 1H), 4.11-4.20 (m, 1H), 3.85-3.96 (m, 1H), 3.70-3.79 (m, 1H), 3.61-3.69 (m, 1H), 3.53-3.61 (m, 1H), 3.45 (d, J=3.1 Hz, 1H), 2.92-2.97 (m, 3H), 2.52-2.57 (m, 3H); ESI-MS [M+H]⁺ calcd for C₂₂H₂₆N₄O₆H⁺443.19 found 443.3.

### EXAMPLE 45

### 3′-chloro-4′-((R)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide

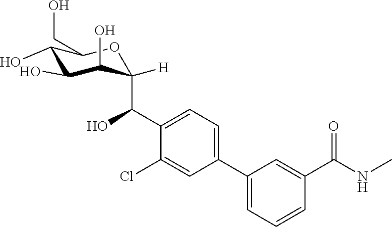

Following Scheme B, intermediate 112R and commercially available N-methyl-3-(4,4,5,5-tetramethyl-[1,3,2]dioxaborolan-2-yl)-benzamide were reacted via the standard Suzuki coupling procedure (2 h at 80° C.), followed by benzyl deprotection protocol D, which requires acetylation, followed by benzyl removal by protocol B (2 h at −78° C.), and acetate deprotection protocol E (2 h, at rt). The resulting residue was purified by HPLC (C18, 15*150 mm column; eluent: acetonitrile/water (0.05% TFA) to give the title compound in 51% yield.

Formula: C₂₁H₂₄C₁NO₇ Exact Mass: 437.12 Molecular Weight: 437.87

¹H NMR (400 MHz, methanol-d₄) δ ppm 7.97 (s, 1H), 7.70 (t, J=9.2 Hz, 2H), 7.53-7.65 (m, 3H), 7.41-7.48 (m, 1H), 5.36 (d, J=7.0 Hz, 1H), 4.13-4.17 (m, 1H), 4.03 (dd, J=7.2, 1.8 Hz, 1H), 3.92 (dd, J=8.8, 3.3 Hz, 1H), 3.69-3.76 (m, 1H), 3.50-3.67 (m, 3H), 2.85 (s, 3H); ESI-MS [M+Na]⁺ calcd for C₂₁H₂₄ClNO₇N⁺460.11 found 460.2.

### EXAMPLE 46

### 4′-((R)-hydroxy((2R,3S,4S,5S,6R)-3,4,5-trihydroxy-6-(hydroxymethyl)tetrahydro-2H-pyran-2-yl)methyl)-3′-methylbiphenyl-4-carbonitrile

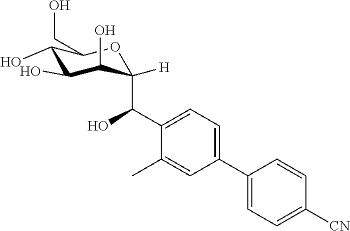

Steps 1-2

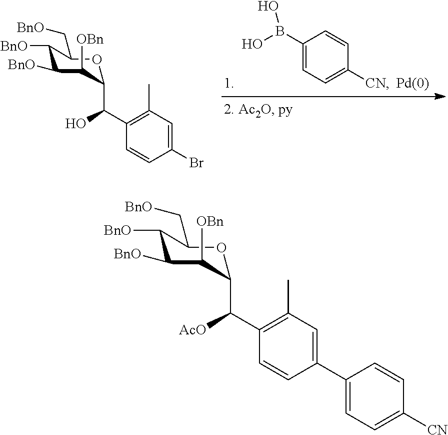

Following Scheme B, intermediate 101R and commercially available 4-cyanophenylboronic acid were reacted via the standard Suzuki coupling procedure (1.75 h at 80° C.), followed by protection of the benzylic alcohol as its acetate (3 h at rt). Purification by column chromatography on silica gel, using (EtOAc-hexanes gradient elution) gave the intermediate shown above in 41% yield (2 steps).

Formula: C₅₁H₄₉NO₇ Exact Mass: 787.35 Molecular Weight: 787.95

¹H NMR (400 MHz, chloroform-d₃) δ ppm 7.60 (d, J=8.2 Hz, 2H), 7.48-7.53 (m, 2H), 7.11-7.25 (m, 23H), 6.11 (d, J=7.0 Hz, 1H), 4.69 (d, J=11.3 Hz, 1H), 4.42-4.57 (m, 5H), 4.33-4.38 (m, 1H), 4.21-4.31 (m, 2H), 3.87-3.92 (m, 1H), 3.82 (t, J=7.6 Hz, 1H), 3.76 (br. s., 1H), 3.72 (br. s., 1H), 3.51-3.63 (m, 2H), 2.38 (s, 3H), 1.81 (s, 3H); ESI-MS [M+Na]⁺ calcd for C₅₁H₄₉NO₇Na⁻ 810.34 found 810.5.

Steps 3-4

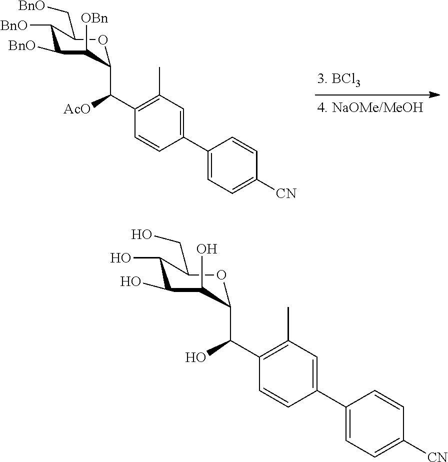

Next, the benzyl groups were removed following deprotection protocol B (30 min at −78° C.), followed by acetate deprotection protocol A (2 h at rt). The resulting residue was purified by HPLC (C18, 15*150 mm column; eluent: acetonitrile/water (0.05% TFA) to give the title compound in 99% yield (40% yield over 4 steps).

Formula: C₂₁H₂₃NO₆ Exact Mass: 385.15 Molecular Weight: 385.42

¹H NMR (400 MHz, methanol-d₄) δ ppm 7.64-7.72 (m, 4H), 7.54 (d, J=8.2 Hz, 1H), 7.35-7.46 (m, 2H), 5.14 (d, J=7.0 Hz, 1H), 4.13 (d, J=2.3 Hz, 1H), 3.90-4.04 (m, 2H), 3.49-3.63 (m, 4H), 2.40 (s, 3H); ESI-MS [M+Na]⁺ calcd for C₂₁H₂₃NO₆Na⁻ 408.14 found 408.3.

### EXAMPLE 47

### (2R,3S,4S,5S,6R)-2-((R)-(4-azido-2-methylphenyl)(hydroxy)methyl)-6-(hydroxymethyl)tetrahydro-2H-pyran-3,4,5-triol

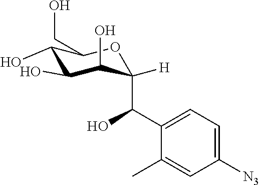

Step 1

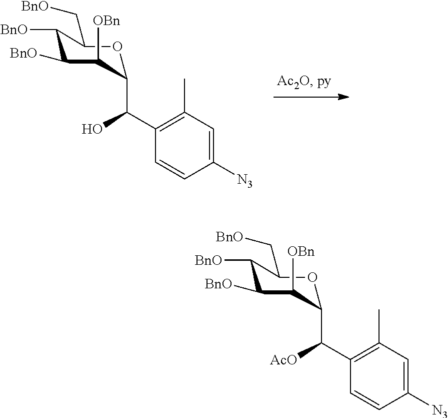

In the first step, the benzylic functionality of Intermediate 113R was protected as its acetate (3 h at rt). Purification by column chromatography on silica gel, using (EtOAc-hexanes gradient elution) gave the acetylated intermediate shown above in 94% yield.

Formula: C₄₄H₄₅N₃O₇ Exact Mass: 727.33 Molecular Weight: 727.86

¹H NMR (400 MHz, chloroform-d₃) δ ppm 7.11-7.29 (m, 20H), 6.60-6.72 (m, 2H), 6.04 (d, J=7.4 Hz, 1H), 4.68 (d, J=11.0 Hz, 1H), 4.32-4.57 (m, 7H), 4.19-4.31 (m, 2H), 3.77-3.89 (m, 2H), 3.62-3.72 (m, 2H), 3.55-3.62 (m, 1H), 3.48-3.54 (m, 1H), 2.27 (s, 3H), 1.78 (d, J=2.0 Hz, 3H); ESI-MS [M+Na]⁺ calcd for C₄₄H₄₅N₃O₇Na⁺750.32 found 750.5.

Steps 2-3

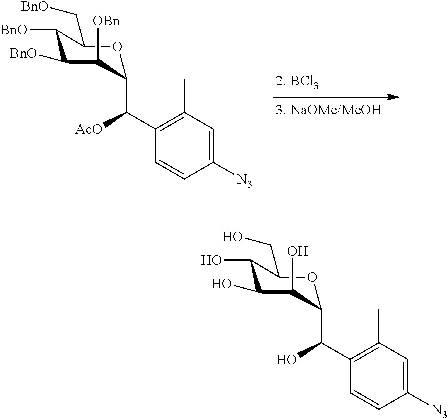

In a second step, the benzyl groups were removed with BCl₃ (1 h at −78° C.), followed in a third step by acetate deprotection protocol A (2 h at rt). The resulting residue was purified by HPLC (C18, 15*150 mm column; eluent: acetonitrile/water (0.05% TFA) to give the title compound in 56% yield (53% yield over 4 steps).

Formula: C₁₄H₁₉N₃O₆ Exact Mass: 325.13 Molecular Weight: 325.32

¹H NMR (400 MHz, methanol-d₄) δ ppm 7.42 (d, J=8.2 Hz, 1H), 6.81 (d, J=8.2 Hz, 1H), 6.75 (s, 1H), 5.02-5.08 (m, 1H), 4.09 (d, J=2.7 Hz, 1H), 3.92-3.98 (m, 1H), 3.88 (dd, J=8.4, 2.9 Hz, 1H), 3.46-3.63 (m, 4H), 2.31 (s, 3H); ESI-MS [M+Na]⁺ calcd for C₁₄H₁₉N₃₀₆Na⁺348.12 found 348.3.

### EXAMPLE 48

### (2R,3S,4S,5S,6R)-2-((R)-hydroxy(2-methyl-4-(4-phenyl-1H-1,2,3-triazol-1-yl)phenyl)methyl)-6-(hydroxymethyl)tetrahydro-2H-pyran-3,4,5-triol

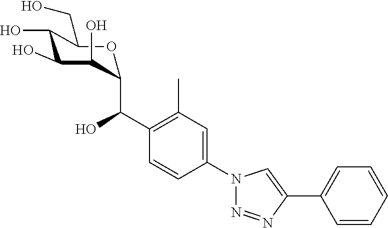

Step 1

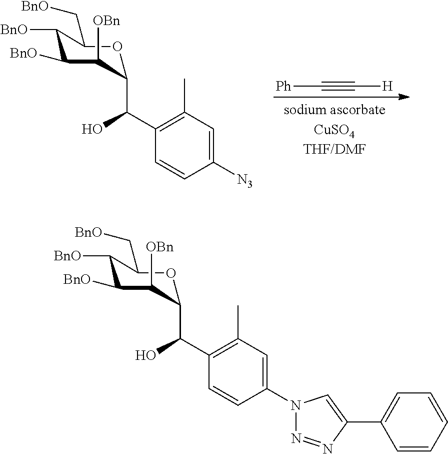

In the first step, intermediate 113R (51 mg, 0.074 mmol) was dissolved into into (2:1, v/v) THF/DMF (3 mL) under nitrogen atmosphere, and phenylacetylene (32 μL, 0.30 mmol) was added dropwise. Sodium ascorbate (5.9 mg, 0.030 mmol) dissolved into H₂O (0.5 mL) was added dropwise, followed by the dropwise addition of CuSO₄-5H₂O (3.7 mg, 0.015 mmol) dissolved into H₂O (0.5 mL) and the reaction was stirred for 20 h at 50° C. Upon completion, the reaction mixture was diluted with 1N HCl (5 mL), and extracted with (1:1, v/v) Et₂O:EtOAc (3×5 mL). The organic layers were combined, and washed again with NH₄Cl (1×5 mL) then H₂O (1×5 mL). The organic layer was evaporated under reduced pressure, and the residue was purified by column chromatography on silica gel (EtOAc-hexanes gradient elution) to give the benzylated intermediate product in 81% yield.

Formula: C₅₀H₄₉N₃O₆ Exact Mass: 787.36 Molecular Weight: 787.96 ¹H NMR (400 MHz, chloroform-d₃) δ ppm 7.99 (s, 1H), 7.89 (d, J=7.4 Hz, 2H), 7.58 (d, J=8.2 Hz, 1H), 7.12-7.51 (m, 25H), 5.12 (d, J=6.3 Hz, 1H), 4.51-4.58 (m, 4H), 4.40-4.46 (m, 1H), 4.28-4.37 (m, 3H), 4.12 (t, J=5.9 Hz, 2H), 4.01 (t, J=4.7 Hz, 2H), 3.68-3.81 (m, 2H), 3.63 (dd, J=10.6, 4.3 Hz, 1H), 2.38 (s, 3H); ESI-MS [M+H]⁺ calcd for C₅₀H₄₉N₃O₆H⁺ 788.37 found 788.6.

Step 2

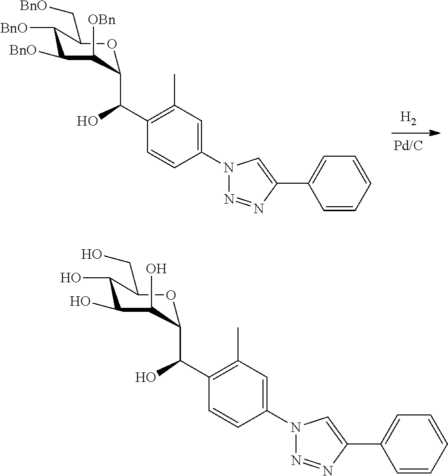

In the second step, deprotection of the benzyl ether was accomplished via deprotection protocol C (20 h at rt). The resulting residue was purified by HPLC (C18, 15*150 mm column; eluent: acetonitrile/water (0.05% TFA) to give the title compound in 52% yield (42% yield over 2 steps).

Formula: C₂₂H₂₅N₃O₆ Exact Mass: 427.17 Molecular Weight: 427.46 ¹H NMR (400 MHz, methanol-d₄) δ ppm 8.77 (s, 1H), 7.82 (d, J=8.2 Hz, 1H), 7.59-7.69 (m, 2H), 7.34-7.42 (m, 1H), 7.23-7.32 (m, 1H), 5.16 (d, J=6.7 Hz, 1H), 4.10-4.15 (m, 1H), 4.02 (dd, J=6.5, 2.2 Hz, 1H), 3.89-3.96 (m, 1H), 3.51-3.65 (m, 3H), 2.45 (s, 2H); ESI-MS [M+H]⁺ calcd for C₂₂H₂₅N₃O₆H⁺428.18 found 428.3.

### EXAMPLE 49

### (2R,3S,4S,5S,6R)-2-((R)-hydroxy(2-methyl-4-(4-(pyridin-3-yl)-1H-1,2,3-triazol-1-yl)-phenyl)methyl)-6-(hydroxymethyl)tetrahydro-2H-pyran-3,4,5-triol

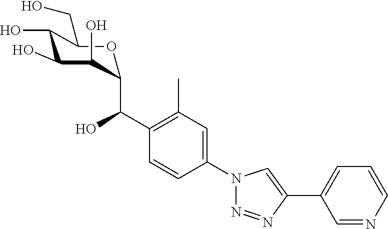

Steps 1-2

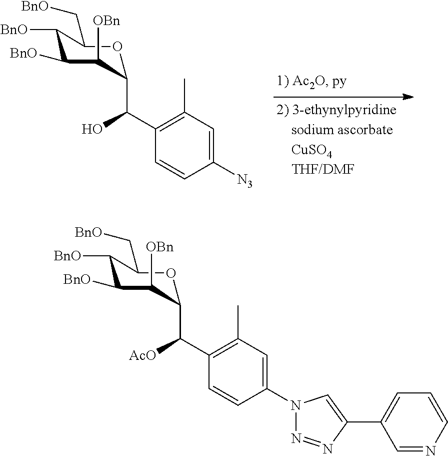

Intermediate 113R was first acetylated, yielding the same intermediate as reported in the first step in the synthesis of Example 47. This acetylated azide intermediate was then reacted in a second step with 3-ethynylpyridine, following the click procedure specified in the synthesis of Example 48. Purification by column chromatography on silica gel, using (EtOAc-hexanes gradient elution) gave to give the protected triazole intermediate in 58% yield.

Formula: C₅₁H₅₀N₄O₇ Exact Mass: 830.37 Molecular Weight: 830.98 ¹H NMR (400 MHz, chloroform-d₃) δ ppm 9.07 (br. s., 1H), 8.61 (d, J=2.7 Hz, 1H), 8.30 (d, J=7.8 Hz, 1H), 8.12 (s, 1H), 7.47-7.52 (m, 2H), 7.43 (d, J=7.8 Hz, 2H), 7.18-7.35 (m, 20H), 6.19 (d, J=7.0 Hz, 1H), 4.74 (d, J=11.3 Hz, 1H), 4.51-4.65 (m, 5H), 4.41-4.47 (m, 1H), 4.33-4.39 (m, 2H), 3.94-4.01 (m, 1H), 3.87 (t, J=7.0 Hz, 1H), 3.81 (br. s., 2H), 3.60-3.73 (m, 2H), 2.48 (s, 3H), 1.92 (s, 3H); ESI-MS [M+H]⁺ calcd for C₅₁H₅₀N₄O₇H⁺ 831.38 found 831.6.

Steps 3-4

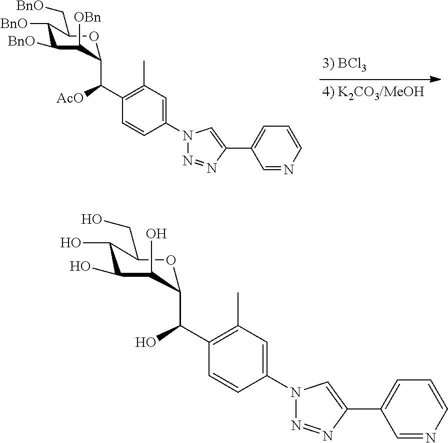

In the third step, benzyl ethers were removed with deprotection protocol B (80 min, −78° C.). In the fourth step, the acetate groups were removed with deprotection protocol E (4.5 h at rt). The resulting residue was purified by HPLC (C18, 15*150 mm column; eluent: acetonitrile/water (0.05% TFA) to give the title compound in 51% yield (30% yield from acetylated azide).

Formula: C₂₁H₂₄N₄O₆ Exact Mass: 428.17 Molecular Weight: 428.45

¹H NMR (400 MHz, methanol-d₄) δ ppm 9.17 (s, 1H), 9.07 (s, 1H), 8.58-8.74 (m, 2H), 7.79-7.88 (m, 1H), 7.64 (d, J=8.2 Hz, 3H), 5.17 (d, J=6.7 Hz, 1H), 4.13 (br. s., 1H), 4.02 (d, J=6.7 Hz, 1H), 3.92 (br. s., 1H), 3.59 (d, J=1.6 Hz, 4H), 2.46 (s, 3H); ESI-MS [M+H]⁺ calcd for C₂₁H₂₄N₄O₆H⁺429.18 found 429.3.

### EXAMPLE 50

### (2R,3S,4S,5S,6R)-2-((1R)-hydroxy(2-methyl-4-(4-(piperidin-3-yl)-1H-1,2,3-triazol-1-yl)-phenyl)methyl)-6-(hydroxymethyl)tetrahydro-2H-pyran-3,4,5-triol

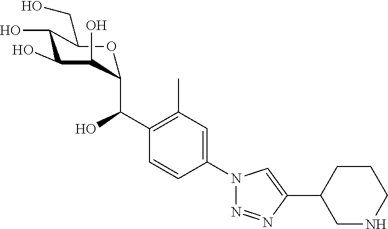

The procedure of Example 48 was followed, employing 3-ethynylpiperidine, followed by deprotection protocol C (16 h at rt). The resulting residue was purified by HPLC (C18, 15*150 mm column; eluent: acetonitrile/water (0.05% TFA) to afford 42% yield of the title compound.

Formula: C₂₁H₃₀N₄O₆ Exact Mass: 434.22 Molecular Weight: 434.49 ¹H NMR (400 MHz, methanol-d₄) δ ppm 8.46 (s, 1H), 7.69-7.77 (m, 1H), 7.61-7.69 (m, 2H), 5.24 (d, J=7.0 Hz, 1H), 4.17-4.24 (m, 1H), 4.10 (dd, J=6.8, 2.5 Hz, 1H), 4.01 (dd, J=7.4, 3.1 Hz, 1H), 3.60-3.74 (m, 5H), 3.43 (d, J=11.3 Hz, 1H), 3.32-3.36 (m, 1H), 3.20-3.28 (m, 1H), 3.03-3.14 (m, 1H), 2.54 (s, 3H), 2.26 (d, J=7.8 Hz, 1H), 2.00-2.14 (m, 1H), 1.82-1.97 (m, 2H); ESI-MS [M+H]⁺ calcd for C₂₁H₃₀N₄O₆H⁺ 435.22 found 435.4.

### EXAMPLE 51

### 4′-((R)-hydroxy((2R,3S,4S,5S,6R)-3,4,5-trihydroxy-6-(hydroxymethyl)tetrahydro-2H-pyran-2-yl)methyl)-3′-methylbiphenyl-3-carbonitrile

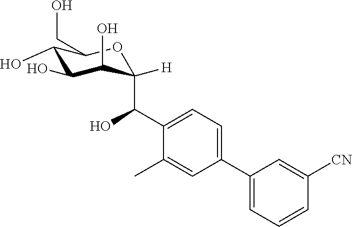

Steps 1-2

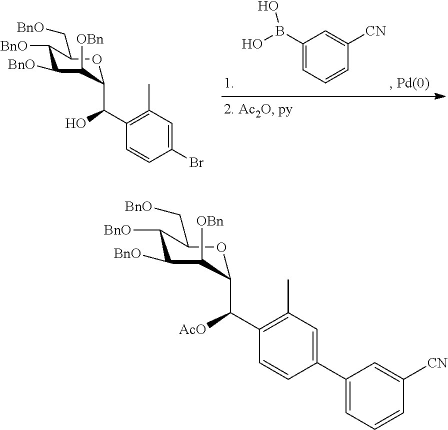

The first and second steps of Example 46 were followed, employing commercially available 3-cyanophenylboronic acid, to afford the intermediate shown above in 46% yield.

Formula: C₅₁H₄₉NO₇ Exact Mass: 787.35 Molecular Weight: 787.95

¹H NMR (400 MHz, chloroform-d₃) δ ppm 7.61-7.69 (m, 2H), 7.49-7.56 (m, 1H), 7.38-7.47 (m, 1H), 7.12-7.28 (m, 23H), 6.09-6.19 (m, 1H), 4.71 (dd, J=10.6, 5.9 Hz, 1H), 4.42-4.56 (m, 5H), 4.32-4.39 (m, 1H), 4.23-4.32 (m, 2H), 3.90 (d, J=5.1 Hz, 1H), 3.70-3.86 (m, 3H), 3.52-3.66 (m, 2H), 2.39 (s, 3H), 1.82 (s, 3H); ESI-MS [M+Na]⁺ calcd for C₅₁H₄₉NO₇Na⁺ 810.34 found 810.5.

Steps 3-4

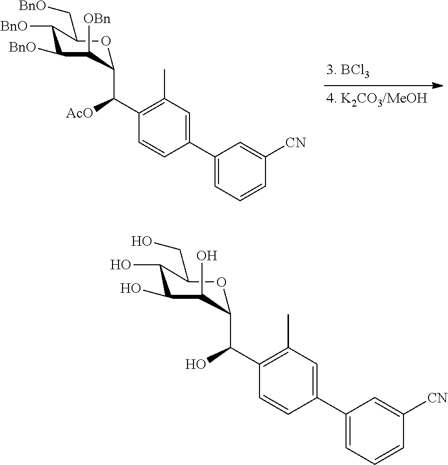

In the third step, benzyl ethers were removed with deprotection protocol B (3 h at −78° C.). In the fourth step, the acetate groups were removed with deprotection protocol E (2 h at rt), to give the title compound in 57% yield (26% yield over 4 steps).

Formula: C₂₁H₂₃NO₆ Exact Mass: 385.15 Molecular Weight: 385.42

¹H NMR (400 MHz, methanol-d₄) δ ppm 7.89-7.98 (m, 2H), 7.56-7.71 (m, 3H), 7.42-7.52 (m, 2H), 5.24 (d, J=6.7 Hz, 1H), 4.24 (d, J=2.3 Hz, 1H), 4.10 (dd, J=6.7, 2.0 Hz, 1H), 4.04 (d, J=4.3 Hz, 1H), 3.63-3.75 (m, 4H), 2.50 (s, 3H); ESI-MS [M+Na]⁺ calcd for C₂₁H₂₃NO₆Na⁻ 408.14 found 408.3.

The following compounds can generally be made using the methods described above. It is expected that these compounds when made will have activity similar to those that have been prepared.

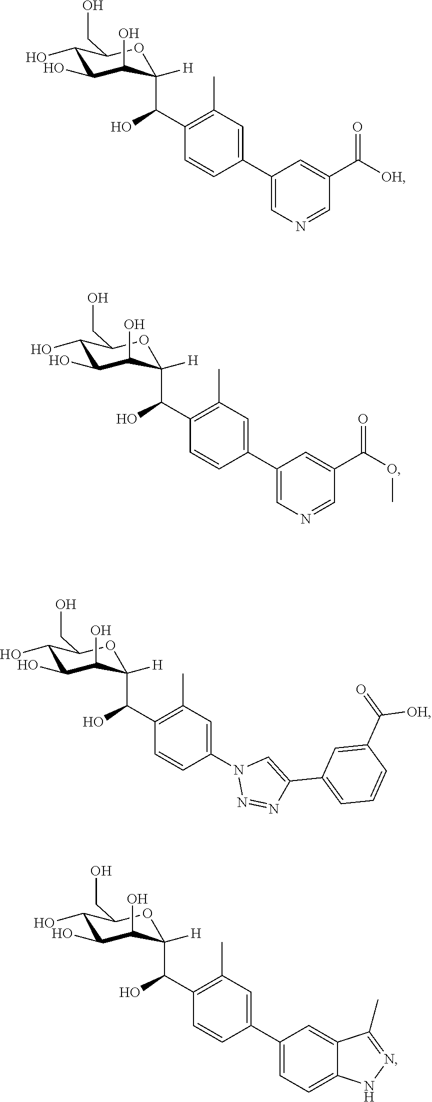

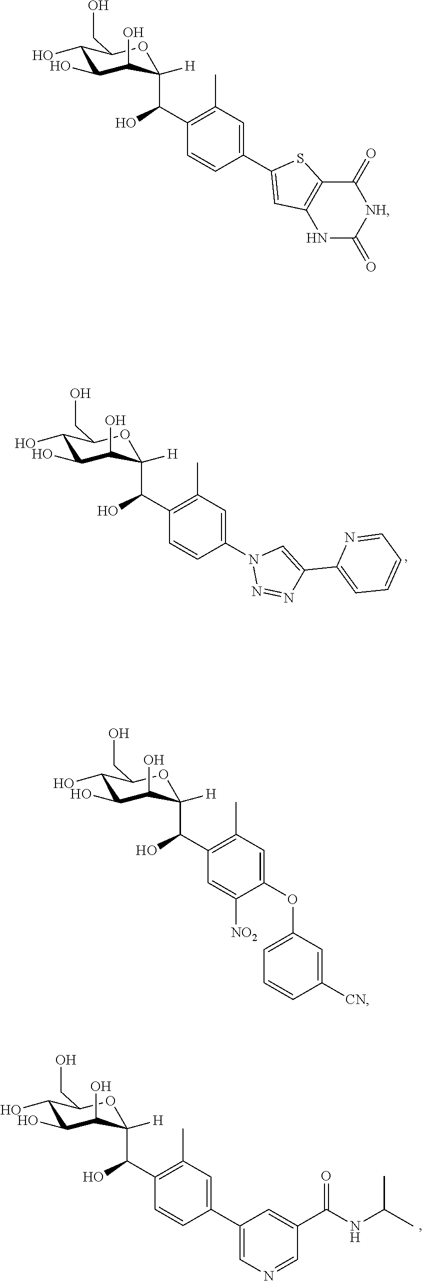

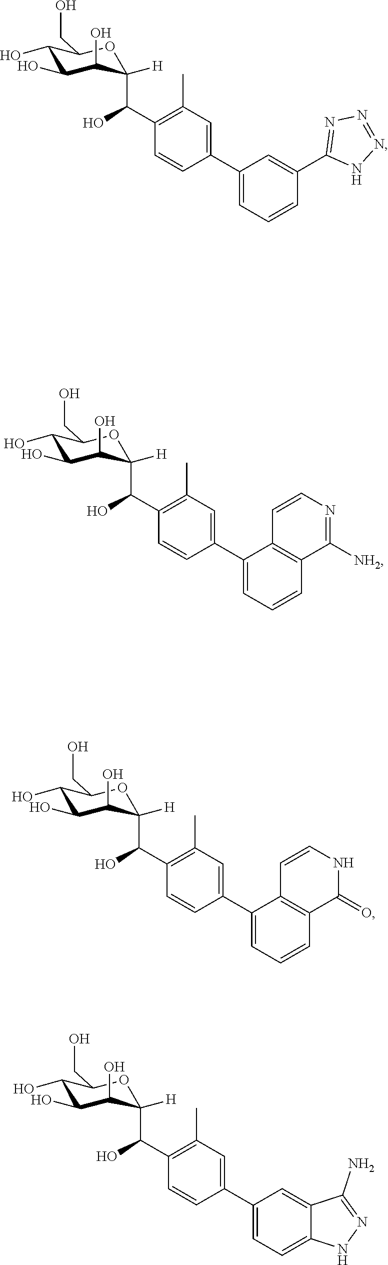

### Biological Activity Assays

The activity of the compounds in Examples 1-51 as FimH inhibitors/antagonists is illustrated in the following assays. The other compounds listed above, which have not yet been made and/or tested, are predicted to have activity in these assays as well.

Hemagglutination Inhibition

The hemagglutination inhibition (HAI) assay was performed with UTI89 bacteria and guinea pig red blood cells, as previously described (S. J. Hultgren, W. R. Schwan, A. J. Schaeffer, J. L. Duncan Infect. Immun. 1986, 54, 613-620). The results are shown in Table 1.

TABLE 1

| | HAI titer |
|---|---|
| Example # | EC_{>90}, (μM) |
| 1 | 0.125 |
| 2 | 0.006 |
| 3 | 0.006 |
| 4 | 0.008 |
| 5 | 1 |
| 6 | 0.5 |
| 7 | 2 |
| 8 | 0.5 |
| 9 | 1.5 |
| 10 | 0.052 |
| 11 | 0.011 |
| 12 | 0.008 |
| 13 | 0.083 |
| 14 | 0.032 |
| 15 | 0.015 |
| 16 | 0.042 |
| 17 | 0.037 |
| 18 | 0.006 |
| 19 | 0.016 |
| 20 | 0.068 |
| 21 | 0.016 |
| 22 | 0.027 |
| 23 | 0.008 |
| 24 | 0.032 |
| 25 | 0.006 |
| 26 | 0.039 |
| 27 | 0.016 |
| 28 | 0.027 |
| 29 | 0.108 |
| 30 | 0.406 |
| 31 | 0.006 |
| 32 | 0.006 |
| 33 | 0.006 |
| 34 | 3 |
| 35 | 0.01 |
| 36 | 0.019 |
| 37 | 0.012 |
| 38 | 0.007 |
| 39 | 0.015 |
| 40 | 5 |
| 41 | 0.057 |
| 42 | 0.006 |
| 43 | 0.006 |
| 44 | 0.062 |
| 45 | 0.047 |
| 46 | 0.053 |
| 47 | 0.25 |
| 48 | 0.016 |
| 49 | 0.015 |
| 50 | NA |
| 51 | 0.026 |

Biofilm Assay

The biofilm inhibition assay was performed with UTI89 bacteria as previously described (L. Cegelski, J. S. Pinkner, N. D. Hammer, C. K. Cusumano, C. S. Hung, E. Chorell, V. Aberg, J. N. Walker, P. C. Seed, F. Almqvist, M. R. Chapman, S. J. Hultgren Nature Chem. Biol. 2009, 5, 913-919). The results are shown in Table 2. Compounds not listed were not tested.

TABLE 2

| | Biofilm prevention |
|---|---|
| Example # | IC₅₀ (μM) |
| 1 | 0.60 |
| 2 | 0.03 |
| 3 | 0.04 |
| 4 | 0.04 |

Differential Scanning Fluorimetry (DSF)

Purified FimH_{L} (10 μM) in the absence or presence of mannoside (100 μM) was combined with 5×SYPRO Orange in a 50 μl reaction mixture buffered in 20 mM HEPES pH 7.5, 150 mM NaCl (HBS) and 0.4% DMSO. Binding equilibria were established by allowing the reaction mixtures to incubate at 23° C. for 30 min. These reaction mixtures were then placed in 96-well clear-bottom PCR plates and subjected to a melt curve from 20-90° C. in 0.5° C. increments of 15 seconds, each followed by a fluorescence read of the “HEX” channel in a Bio-Rad CFX96 thermocycler (Bio-Rad, Hercules, Calif.). Melt curves were fitted to the Boltzmann equation (y=A2+(A1−A2)/(1+exp((x−xₒ)/dx)) where xₒ is the Tₘ) to determine the melting temperature (Tₘ) using GraphPad Prism 6 (San Diego Calif.). Melting temperatures are represented as the mean and standard error of two biological replicates, each of which consisted of three technical replicates. The results are shown in Table 3. Compounds not listed were not tested.

TABLE 3

| Example # | DSF Melting Temp (° C.) |
|---|---|
| 1 | 74.6 |
| 2 | 77.5 |
| 3 | 77.7 |
| 4 | 76.06 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of Formula (I): 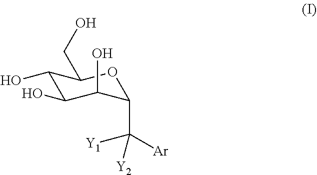
wherein:
Ar is aryl or heteroaryl;
wherein:
each aryl and heteroaryl as defined for Ar is substituted with W and one or two Z groups;
wherein:
Z is lower alkyl, lower haloalkyl, NO₂, CF₃, cyclopropyl, lower alkoxy, halo, hydroxyl, and amino;
wherein:
amino as defined for Z is optionally substituted with one or two lower alkyl,
W is aryl, heteroaryl, or azide;
wherein:
aryl or heteroaryl as defined for W is substituted with one or more substituents selected from R₁₁, H, boronic acid, boronic acid pinacol ester, alkyl, OTf, hydroxyl, amino optionally substituted with one or two alkyl or aryl groups, azide, alkyne, —SO₂Aryl; —C(O)OR₅, C(O)NR₈R₉, halo, OCF₃, alkenyl, alkynyl, haloalkyl, CN, alkoxy, NHSO₂R₆, NHSO₂NHR₆, NHCOR₆, NHCONHR₆, and cycloalkyl, heterocycloalkyl, aryl, aryloxy, aralkyl, and heteroaryl any of which may be optionally substituted with one or more alkyl, hydroxyl, oxo, CN, and NR₈R₉,
wherein:
each R₅ and R₆ independently is hydrogen, alkyl, cycloalkyl, heterocycloalkyl, aryl, heteroaryl, aralkyl and heteroaralkyl;
each R₈ and R₉ is independently hydrogen, C₁-C₆ alkyl, aryl, heteroaryl, aralkyl, and heteroaralkyl; or
R₈ and R₉ taken together form a heterocycloalkyl;
R₁₁ is halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, NHSO₂R₁₂, NHSO₂NHR₁₂, NHCOR₁₂, NHCONHR₁₂, CONHR₁₂, CONR₁₂ₐR_{12b}, hydroxy, and OCF₃;
wherein:
each R₁₂, R₁₂ₐ and R_{12b} independently is selected from hydrogen, C₁-C₆ alkyl, aryl, heteroaryl, aralkyl and heteroaralkyl;
each Y₁ and Y₂ independently is selected from H, hydroxyl, lower alkoxy or amino;
wherein:
each amino as defined for each Y₁ and Y₂ is optionally substituted with one or two lower alkyl, cyano, azide, nitro, haloalkyl, halo, haloalkoxy; and acetyl;
provided that:
the compound of Formula (I) is not:
3-[4-[(R)-hydroxy-[(2R,3S,4S,5S,6R)-3,4,5-trihydroxy-6-(hydroxymethyl)tetrahydropyran-2-yl]methyl]-3-methyl-phenyl]-N-methylbenzamide,
3-[4-[(S)-hydroxy-[2R, 3S,4S,5S,6R)-3,4,5-trihydroxy-6-(hydroxymethyl)tetrahydropyran-2-yl]methyl]-3-methyl-phenyl]-N-methylbenzamide,
N-methyl-3-[3-methyl-4-[[(2R,3S,4R,5S,6R)-3,4,5-trihydroxy-6-(hydroxy-methyl)tetrahydro-pyran-2-yl]methyl]phenyl]benzamide,
4′-((R)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide,
N-methyl-4′-(((2R,3S,4R,5S,6R)-3,4,5-trihydroxy-6-(hydroxymethyl)tetrahydro-2H-pyran-2-yl)methyl)biphenyl-3-carboxamide,
4′-((S)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide,
N,3′-dimethyl-4′-(((2R,3S,4R,5S,6R)-3,4,5-trihydroxy-6-(hydroxymethyl)tetrahydro-2H-pyran-2-yl)methyl)biphenyl-3-carboxamide,
N-methyl-3′-(trifluoromethyl)-4′-(((2R,3S,4R,5S,6R)-3,4,5-trihydroxy-6-(hydroxymethyl)tetrahydro-2H-pyran-2-yl)methyl)biphenyl-3-carboxamide,
3′-chloro-N-methyl-4′-(((2R,3S,4R,5S,6R)-3,4,5-trihydroxy-6-(hydroxymethyl)tetrahydro-2H-pyran-2-yl)methyl)biphenyl-3-carboxamide,
3′-fluoro-N-methyl-4′-(((2R,3S,4R,5S,6R)-3,4,5-trihydroxy-6-(hydroxymethyl)tetrahydro-2H-pyran-2-yl)methyl)biphenyl-3-carboxamide,
3′-methoxy-N-methyl-4′-(((2R,3S,4R,5S,6R)-3,4,5-trihydroxy-6-(hydroxymethyl)tetrahydro-2H-pyran-2-yl)methyl)biphenyl-3-carboxamide,
N³,N⁵-dimethyl-4′-(((2R,3S,4R,5 S,6R)-3,4,5-trihydroxy-6-(hydroxymethyl)tetrahydro-2H-pyran-2-yl)methyl)biphenyl-3,5-dicarboxamide,
N³,N⁵,3′-trimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide,
N³,N⁵-dimethyl-3′-(trifluoromethyl)-4′-(((2R,3 S,4R,5 S,6R)-3,4,5-trihydroxy-6-(hydroxymethyl)tetrahydro-2H-pyran-2-yl)methyl)biphenyl-3,5-dicarboxamide,
3′-chloro-N³,N⁵-dimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide,
3′-fluoro-N³,N-dimethyl-4′-(((2R,3S,4R,5 S,6R)-3,4,5-trihydroxy-6-(hydroxymethyl)tetrahydro-2H-pyran-2-yl)methyl)biphenyl-3,5-dicarboxamide,
3′-methoxy-N³,N⁵-dimethyl-4′-(((2R,3S,4R,5S,6R)-3,4,5-trihydroxy-6-(hydroxymethyl)tetrahydro-2H-pyran-2-yl)methyl)biphenyl-3,5-dicarboxamide.

2. The compound of Formula (I) according to claim 1, wherein Y₁ is hydrogen, and the stereochemistry at C-1 is R configuration.

3. A compound of Formula (II): 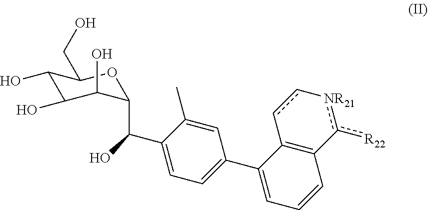
wherein:
“----” represents a single or double bond;
R₂₁ is null, hydrogen or lower alkyl;
R₂₂ is hydrogen, alkyl, hydroxyl, O or NR₂₈R₂₉;
wherein:
each R₂₈ and R₂₉ independently is hydrogen, C₁-C₆ alkyl, aryl, heteroaryl, aralkyl or heteroaralkyl; or
R₂₈ and R₂₉ taken together form a heterocycloalkyl.

4. A compound of Formula (III): 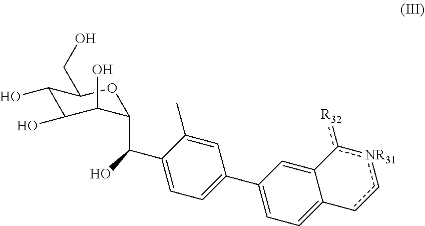
wherein:
“----” represents a single or double bond;
R₃₁ is null, hydrogen or lower alkyl;
R₃₂ is hydrogen, alkyl, hydroxyl, O or NR₃₈R₃₉;
wherein:
each R₃₈ and R₃₉ independently is hydrogen, C₁-C₆ alkyl, aryl, heteroaryl, aralkyl, and heteroaralkyl; or
R₃₈ and R₃₉ taken together form a heterocycloalkyl.

5. A compound of Formula (IV): 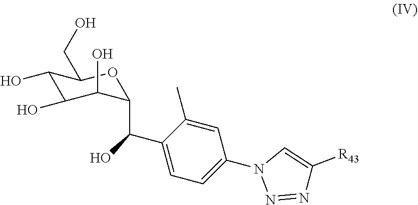
wherein:
R₄₃ is alkyl, cycloalkyl, heterocycloalkyl, aryl or heteroaryl;
wherein:
each alkyl, cycloalkyl, heterocycloalkyl, aryl or heteroaryl as defined for R₄₃ is optionally substituted with one or more substituents selected from hydrogen, halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, COOR₄₄, NHSO₂R₄₄, NHSO₂NHR₄₄, NHCOR₄₄, NHCONHR₄₄, CONHR₄₄, CONR₄₄ₐR_{44b}, hydroxy, or OCF₃;
wherein:
each R₄₄, R₄₄ₐ and R_{44b} independently is selected from hydrogen, C₁-C₆ alkyl, aryl, heteroaryl, aralkyl or heteroaralkyl;

6. A compound of Formula (V): 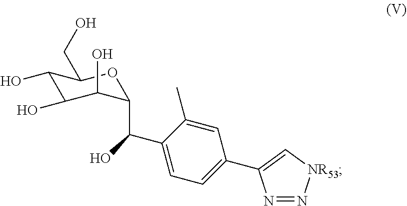
wherein:
R₅₃ is null, hydrogen or lower alkyl; or
a pharmaceutically acceptable salt thereof.

7. A compound of Formula (VI): 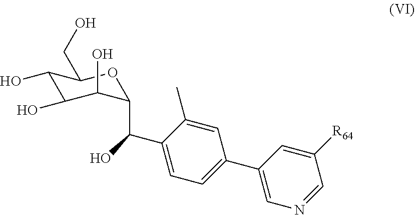
wherein:
R₆₄ is —C(O)OR₆₅, C(O)NR₆₈R₆₉, halo, hydroxy, OCF₃, alkyl, alkenyl, alkynyl, cycloalkyl, haloalkyl, aralkyl, heterocycloalkyl, CN, alkoxy, amino, alkylamino, dialkylamino, NHSO₂R₆₆, NHSO₂NHR₆₆, NHCOR₆₆, NHCONHR₆₆; or aryl or heteroaryl either of which may be optionally substituted with halo, hydroxy, OCF₃, alkyl, alkenyl, alkynyl, haloalkyl, CN, alkoxy, alkylamino, dialkylamino, NHSO₂R₆₆, NHSO₂NHR₆₆, NHCOR₆₆, or NHCONHR₆₆;
wherein:
R₆₅ is hydrogen, alkyl, cycloalkyl, heterocycloalkyl, aryl or heteroaryl;
each R₆₈ and R₆₉ are each independently chosen from hydrogen, C₁-C₆ alkyl, aryl, heteroaryl, aralkyl, and heteroaralkyl, or R₆₈ and R₆₉ taken together form a heterocycloalkyl; and
R₆₆ is hydrogen, C₁-C₆ alkyl, aryl, heteroaryl, aralkyl and heteroaralkyl; or
a pharmaceutically acceptable salt thereof.

8. A compound of Formula (VII): 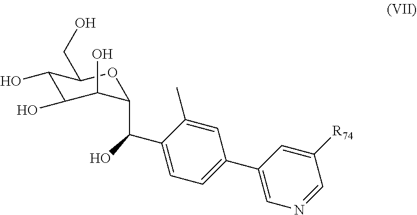
wherein:
R₇₄ is —C(O)OR₇₅, C(O)NR₇₈R₇₉, halo, hydroxy, OCF₃, alkyl, alkenyl, alkynyl, cycloalkyl, haloalkyl, aralkyl, heterocycloalkyl, CN, alkoxy, alkylamino, dialkylamino, NHSO₂R₇₇, NHSO₂NHR₇₇, NHCOR₇₇, NHCONHR₇₇; or aryl or heteroaryl either of which optionally is substituted with halo, hydroxy, OCF₃, alkyl, alkenyl, alkynyl, haloalkyl, CN, alkoxy, alkylamino, dialkylamino, NHSO₂R₇₇, NHSO₂NHR₇₇, NHCOR₇₇ or NHCONHR₇₇;
wherein:
R₇₅ is hydrogen, alkyl, cycloalkyl, heterocycloalkyl, aryl or heteroaryl;
R₇₈ and R₇₉ are each independently chosen from hydrogen, C₁-C₆ alkyl, aryl, heteroaryl, aralkyl, and heteroaralkyl;
R₇₈ and R₇₉ taken together form a heterocycloalkyl; and
R₇₇ is hydrogen, C₁-C₆ alkyl, aryl, heteroaryl, aralkyl or heteroaralkyl.

9. A compound of Formula (VIII): 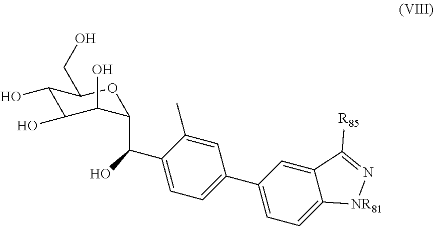
wherein:
R₈₁ is from null, hydrogen, and lower alkyl;
R₈₅ is from hydrogen, alkyl, NR₈₈R₈₉, aryl, heteroaryl, cycloalkyl, and heterocycloalkyl, any of which may be optionally substituted;
each R₈₈ and R₈₉ independently is hydrogen, C₁-C₆ alkyl, aryl, heteroaryl, aralkyl, or heteroaralkyl, or
R₈₈ and R₈₉ taken together form a heterocycloalkyl; or
a pharmaceutically acceptable salt thereof.

10. A compound of Formula (IX): 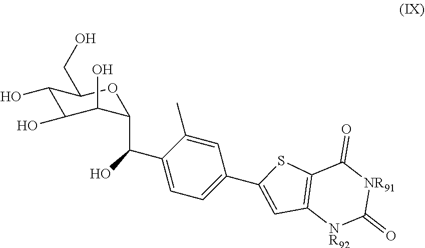
wherein:
each R₉₁ and R₉₂ independently is hydrogen or lower alkyl.

11. A compound of Formula (X): 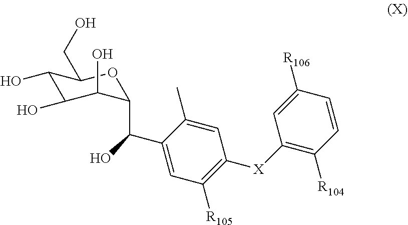
wherein:
R₁₀₆ is from cyano, C(O)NR₁₀₉R₁₁₀, NR₁₀₉R₁₁₀, —SO₂NR₁₁₁R₁₁₂, NHC(O)NR₁₀₉R₁₁₀, nitro, hydroxyl, halo, and heteroaryl;
each R₁₀₉ and R₁₁₀ independently is hydrogen, C₁-C₆ alkyl, aryl, heteroaryl, aralkyl, and heteroaralkyl, or taken together, R₁₀₉ and R₁₁₀ may form a heterocycloalkyl; and
each R₁₁₁ and R₁₁₂ independently is H, C₃-C₇-cycloalkyl; C₂-C₆-alkyl, aryl, or heteroaryl; or
R₁₁₁ and R₁₁₂ together with the atom to which they are attached form a C₃-C₇-heterocycloalkyl or heteroaryl;
each R₁₀₄ and R₁₀₅ independently is hydrogen or nitro; and
X is O, NH, or SO₂; or
a pharmaceutically acceptable salt thereof.

12. A compound of Formula (XI): 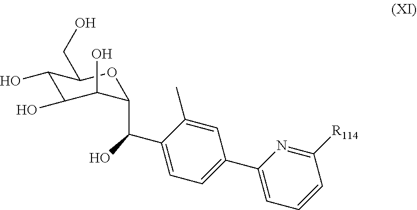
wherein:
R₁₁₄ is —C(O)OR₁₁₅, C(O)NR₁₁₈R₁₁₉, halo, hydroxy, OCF₃, alkyl, alkenyl, alkynyl, cycloalkyl, haloalkyl, aralkyl, heterocycloalkyl, CN, alkoxy, alkylamino, dialkylamino, NHSO₂R₁₁₇, NHSO₂NHR₁₁₇, NHCOR₁₁₇, NHCONHR₁₁₇, and aryl and heteroaryl which may be optionally substituted with halo, hydroxy, OCF₃, alkyl, alkenyl, alkynyl, haloalkyl, CN, alkoxy, alkylamino, dialkylamino, NHSO₂R₁₁₇, NHSO₂NHR₁₁₇, NHCOR₁₁₇, or NHCONHR₁₁₇;
R₁₁₅ is chosen from hydrogen, alkyl, cycloalkyl, heterocycloalkyl, aryl, heteroaryl;
R₁₁₈ and R₁₁₉ are each independently chosen from hydrogen, C₁-C₆ alkyl, aryl, heteroaryl, aralkyl, and heteroaralkyl, or taken together, R₁₁₈ and R₁₁₉ may form a heterocycloalkyl; and
R₁₁₇ is hydrogen, C₁-C₆ alkyl, aryl, heteroaryl, aralkyl and heteroaralkyl; or
a pharmaceutically acceptable salt thereof.

13. A compound of Formula (XII): 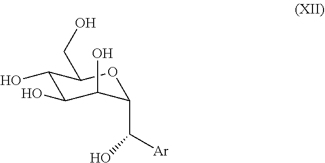
wherein:
Ar is aryl or heteroaryl;
wherein:
each aryl and heteroaryl as defined for Ar is substituted with W and one or two Z groups;
wherein:
Z is lower alkyl, lower haloalkyl, NO₂, CF₃, cyclopropyl, lower alkoxy, halo, hydroxyl, and amino;
wherein:
amino as defined for Z is optionally substituted with one or two lower alkyl,
W is aryl, heteroaryl, or azide;
wherein:
aryl or heteroaryl as defined for W is substituted with one or more substituents selected from R₁₁, H, boronic acid, boronic acid pinacol ester, alkyl, OTf, hydroxyl, amino optionally substituted with one or two alkyl or aryl groups, azide, alkyne, —SO₂Aryl; —C(O)OR₅, C(O)NR₈R₉, halo, OCF₃, alkenyl, alkynyl, haloalkyl, CN, alkoxy, NHSO₂R, NHSO₂NHR₆, NHCOR₆, NHCONHR₆, and cycloalkyl, heterocycloalkyl, aryl, aryloxy, aralkyl, and heteroaryl any of which may be optionally substituted with one or more alkyl, hydroxyl, oxo, CN, and NR₈R₉,
wherein:
each R₅ and R₆ independently is hydrogen, alkyl, cycloalkyl, heterocycloalkyl, aryl, heteroaryl, aralkyl and heteroaralkyl;
each R₈ and R₉ is independently hydrogen, C₁-C₆ alkyl, aryl, heteroaryl, aralkyl, and heteroaralkyl; or
R₈ and R₉ taken together form a heterocycloalkyl;
R₁₁ is halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, NHSO₂R₁₂, NHSO₂NHR₁₂, NHCOR₁₂, NHCONHR₁₂, CONHR₁₂, CONR₁₂ₐR_{12b}, hydroxy, and OCF₃;
wherein:
each R₁₂, R₁₂ₐ and R_{12b} independently is selected from hydrogen, C₁-C₆ alkyl, aryl, heteroaryl, aralkyl and heteroaralkyl;
provided that the compound of Formula (XII) is not
3-[4-[(S)-hydroxy-[2R,3S,4S,5S,6R)-3,4,5-trihydroxy-6-(hydroxymethyl)tetrahydropyran-2-yl]methyl]-3-methyl-phenyl]-N-methylbenzamide, or
4′-((S)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide
or an ester or pharmaceutically acceptable salt thereof.

14. A compound of Formula (XIII): 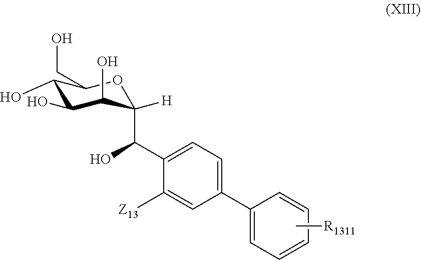
or an ester or pharmaceutically acceptable salt thereof, wherein:
R₁₃₁₁ is chosen from halo, alkyl, alkenyl, alkynyl, cycloalkyl, haloalkyl, aryl, aralkyl, heterocyclyl, heteroaryl, heterarylalkyl, CN, alkoxy, alkylamino, dialkylamino, NHSO₂R₁₃₁₂, NHSO₂NHR₁₃₁₂, NHCOR₁₃₁₂, NHCONHR₁₃₁₂, CONHR₁₃₁₂, CONR₁₃₁₂ₐR_{1312b}, hydroxy, and OCF₃;
R₁₃₁₂, R₁₃₁₂ₐ and R_{1312b} are independently chosen from hydrogen, C₁-C₆ alkyl, aryl, heteroaryl, aralkyl and heteroaralkyl; and
Z₁₃ is chosen from lower alkyl, lower haloalkyl, NO₂, CF₃, cyclopropyl, lower alkoxy, halo, hydroxyl, and amino optionally substituted with one or two lower alkyl, with the proviso that the compound is not
3-[4-[(R)-hydroxy-[(2R,3S,4S,5S,6R)-3,4,5-trihydroxy-6-(hydroxymethyl)tetrahydropyran-2-yl]methyl]-3-methyl-phenyl]-N-methylbenzamide.

15. A compound which is: 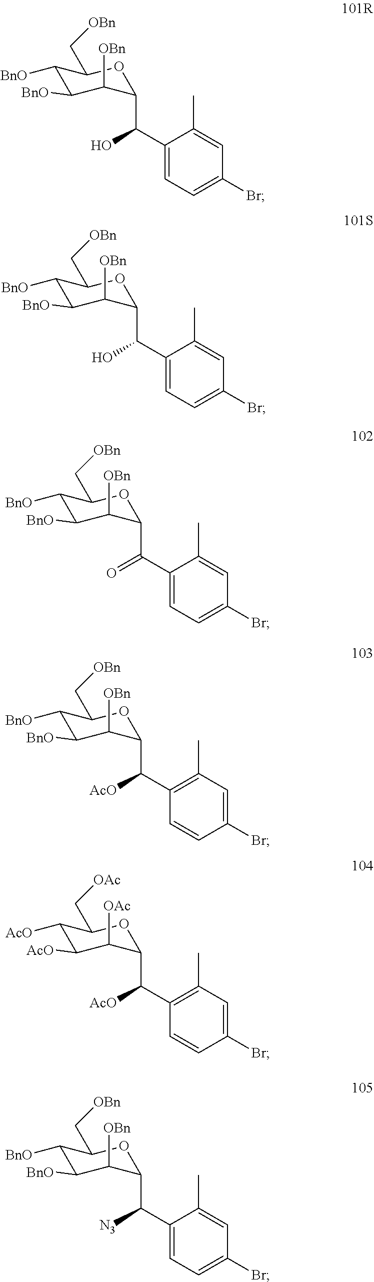 [SMILES: Cc1cc(Br)ccc1[C@@H](O)C1OC(COCc2ccccc2)C(OCc2ccccc2)C(OCc2ccccc2)C1OCc1ccccc1 ; Cc1cc(Br)ccc1[C@H](O)C1OC(COCc2ccccc2)C(OCc2ccccc2)C(OCc2ccccc2)C1OCc1ccccc1 ; Cc1cc(Br)ccc1C(=O)C1OC(COCc2ccccc2)C(OCc2ccccc2)C(OCc2ccccc2)C1OCc1ccccc1 ; CC(=O)O[C@H](c1ccc(Br)cc1C)C1OC(COCc2ccccc2)C(OCc2ccccc2)C(OCc2ccccc2)C1OCc1ccccc1 ; CC(=O)OCC1OC([C@H](OC(C)=O)c2ccc(Br)cc2C)C(OC(C)=O)C(OC(C)=O)C1OC(C)=O ; Cc1cc(Br)ccc1[C@@H](N=[N+]=[N-])C1OC(COCc2ccccc2)C(OCc2ccccc2)C(OCc2ccccc2)C1OCc1ccccc1] 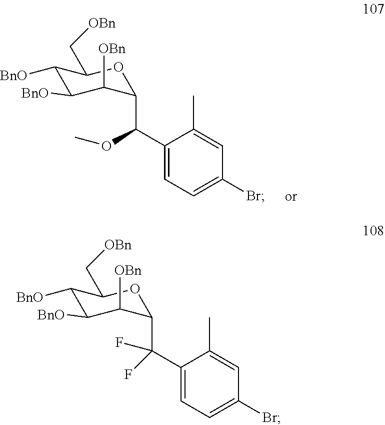 [SMILES: CO[C@H](c1ccc(Br)cc1C)C1OC(COCc2ccccc2)C(OCc2ccccc2)C(OCc2ccccc2)C1OCc1ccccc1 ; Cc1cc(Br)ccc1C(F)(F)C1OC(COCc2ccccc2)C(OCc2ccccc2)C(OCc2ccccc2)C1OCc1ccccc1]
or
a pharmaceutically acceptable salt thereof.

16. A compound which is
(2R,3S,4S,5S,6R)-2-((R)-hydroxy(4-(isoquinolin-5-yl)-2-methylphenyl)methyl)-6-(hydroxymethyl)tetrahydro-2H-pyran-3,4,5-triol,
7-(4-((R)-hydroxy((2R,3S,4S,5S,6R)-3,4,5-trihydroxy-6-(hydroxymethyl)tetrahydro-2H-pyran-2-yl)methyl)-3-methylphenyl)isoquinolin-1 (2H)-one,
(2R,3S,4S,5 S,6R)-2-((R)-(4-(1-aminoisoquinolin-7-yl)-2-methylphenyl)(hydroxy)methyl)-6-(hydroxymethyl)tetrahydro-2H-pyran-3,4,5-triol,
7-(4-((R)-hydroxy((2R,3S,4S,5S,6R)-3,4,5-trihydroxy-6-(hydroxymethyl)tetrahydro-2H-pyran-2-yl)methyl)-3-methylphenyl)-3,4-dihydroisoquinolin-1 (2H)-one,
(2R,3S,4S,5S,6R)-2-((R)-hydroxy(2-methyl-4-(2-methyl-1,2,3,4-tetrahydroisoquinolin-5-yl)phenyl)methyl)-6-(hydroxymethyl)tetrahydro-2H-pyran-3,4,5-triol,
4′-((R)-amino((2R,3S,4R,5S,6R)-3,4,5-trihydroxy-6-(hydroxymethyl)tetrahydro-2H-pyran-2-yl)methyl)-N,3′-dimethylbiphenyl-3-carboxamide,
4′-((R)-methoxy((2S,3S,4S,5S,6R)-3,4,5-trihydroxy-6-(hydroxymethyl)tetrahydro-2H-pyran-2-yl)methyl)-N,3′-dimethylbiphenyl-3-carboxamide,
4′-(methoxy((2S,3S,4S,5S,6R)-3,4,5-trihydroxy-6-(hydroxymethyl)tetrahydro-2H-pyran-2-yl)methyl)-N,2′-dimethylbiphenyl-3-carboxamide,
4′-(difluoro((2S,3S,4S,5S,6R)-3,4,5-trihydroxy-6-(hydroxymethyl)tetrahydro-2H-pyran-2-yl)methyl)-N, 3′-dimethylbiphenyl-3-carboxamide,
(2R,3S,4S,5S,6R)-2-((R)-Hydroxy (4-(isoquinolin-6-yl)-2-methylphenyl)methyl)-6-(hydroxymethyl)-tetrahydro-2H-pyran-3,4,5-triol,
7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-methylisoquinolin-1 (2H)-one,
7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-methyl-3,4-dihydroisoquinolin-1 (2H)-one,
4-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)isoindolin-1-one,
(2R,3S,4S,5S,6R)-2-((R)-Hydroxy (2-methyl-4-(quinolin-6-yl)phenyl)methyl)-6-(hydroxymethyl)-tetrahydro-2H-pyran-3,4,5-triol,
(2R,3S,4S,5S,6R)-2-((R)-Hydroxy (2-methyl-4-(quinolin-7-yl)phenyl)methyl)-6-(hydroxymethyl)-tetrahydro-2H-pyran-3,4,5-triol,
(2R,3S,4S,5S,6R)-2-((R)-Hydroxy (2-methyl-4-(3-methyl-3H-benzo[d]imidazol-5-yl)phenyl)methyl)-6-(hydroxymethyl)-tetrahydro-2H-pyran-3,4,5-triol,
(2R,3S,4S,5S,6R)-2-((R)-(4-(3H-Benzo[d]imidazol-5-yl)-2-methylphenyl) (hydroxy)methyl)-6-(hydroxymethyl)-tetrahydro-2H-pyran-3,4,5-triol,
(2R,3S,4S,5S,6R)-2-((R)-(4-(3-Amino-1H-indazol-7-yl)-2-methylphenyl) (hydroxy)methyl)-6-(hydroxymethyl)-tetrahydro-2H-pyran-3,4,5-triol,
6-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)isoindolin-1-one,
(2R,3S,4S,5S,6R)-2-((R)-Hydroxy (2-methyl-4-(3-methylbenzo[d]isoxazol-5-yl)phenyl)methyl)-6-(hydroxymethyl)-tetrahydro-2H-pyran-3,4,5-triol,
(2R,3S,4S,5S,6R)-2-((R)-Hydroxy (4-(isoquinolin-7-yl)-2-methylphenyl)methyl)-6-(hydroxymethyl)-tetrahydro-2H-pyran-3,4,5-triol,
(2R,3S,4S,5S,6R)-2-((R)-(4-(2-Chloropyridin-4-yl)-2-methylphenyl) (hydroxy)methyl)-6-(hydroxymethyl)-tetrahydro-2H-pyran-3,4,5-triol,
(2R,3S,4S,5S,6R)-2-((R)-(4-(2′-Chloro-[2,4′-bipyridin]-4-yl)-2-methylphenyl) (hydroxy)methyl)-6-(hydroxymethyl)tetrahydro-2H-pyran-3,4,5-triol,
(2R,3S,4S,5S,6R)-2-((R)-Hydroxy (2-methyl-4-(2-(methylamino)pyridin-4-yl)phenyl)methyl)-6-(hydroxymethyl)-tetrahydro-2H-pyran-3,4,5-triol,
(2R,3S,4S,5S,6R)-2-((R)-Hydroxy (2-methyl-4-(2-morpholinopyridin-4-yl)phenyl)methyl)-6-(hydroxymethyl)-tetrahydro-2H-pyran-3,4,5-triol,
(2R,3S,4S,5S,6R)-2-((R)-(4-(2-Aminopyridin-4-yl)-2-methylphenyl) (hydroxy)methyl)-6-(hydroxymethyl)-tetrahydro-2H-pyran-3,4,5-triol,
(2R,3S,4S,5S,6R)-2-((R)-(4-(2-(Dimethylamino)pyridin-4-yl)-2-methylphenyl) (hydroxy)methyl)-6-(hydroxymethyl)-tetrahydro-2H-pyran-3,4,5-triol,
(2R,3S,4S,5S,6R)-2-((R)-(4-(3-Aminobenzo[d]isoxazol-5-yl)-2-methylphenyl) (hydroxy)methyl)-6-(hydroxymethyl)-tetrahydro-2H-pyran-3,4,5-triol,
7-(4-((R)-Hydroxy ((2R,3S,4S,5S,6R)-3,4,5-trihydroxy-6-(hydroxymethyl)-tetrahydro-2H-pyran-2-yl)methyl)-3,5-dimethylphenyl)isoquinolin-1 (2H)-one,
7-(4-((S)-hydroxy ((2R,3S,4S,5S,6R)-3,4,5-trihydroxy-6-(hydroxymethyl)tetrahydro-2H-pyran-2-yl)methyl)-3,5-dimethylphenyl)isoquinolin-1 (2H)-one,
7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-isopropylisoquinolin-1 (2H)-one,
7-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-2-isopropyl-3,4-dihydroisoquinolin-1 (2H)-one,
7-(4-((R)-Hydroxy ((2R,3S,4S,5S,6R)-3,4,5-trihydroxy-6-(hydroxymethyl)-tetrahydro-2H-pyran-2-yl)methyl)-3-(trifluoromethyl)phenyl)-3,4-dihydroisoquinolin-1 (2H)-one,
7-(4-((S)-hydroxy ((2R,3S,4S,5S,6R)-3,4,5-trihydroxy-6-(hydroxymethyl)tetrahydro-2H-pyran-2-yl)methyl)-3-(trifluoromethyl)phenyl)-3,4-dihydroisoquinolin-1 (2H)-one,
5-(4-((R)-Hydroxy ((2R,3S,4S,5S,6R)-3,4,5-trihydroxy-6-(hydroxymethyl)-tetrahydro-2H-pyran-2-yl)methyl)-3-methylphenyl)-N-methylnicotinamide,
(2R,3S,4S,5S,6R)-2-((R)-Hydroxy (4-(imidazo[1,2-a]pyridin-2-yl)-2-methylphenyl)methyl)-6-(hydroxymethyl)-tetrahydro-2H-pyran-3,4,5-triol,
4′-((R)-Hydroxy ((2R,3S,4S,5S,6R)-3,4,5-trihydroxy-6-(hydroxymethyl)-tetrahydro-2H-pyran-2-yl)methyl)-5′-methyl-N-(pyridin-2-yl)biphenyl-3-carboxamide,
4′-((R)-Hydroxy ((2R,3S,4S,5S,6R)-3,4,5-trihydroxy-6-(hydroxymethyl)-tetrahydro-2H-pyran-2-yl)methyl)-N³,N⁵,3′-trimethylbiphenyl-3,5-dicarboxamide,
4′-((R)-Hydroxy ((2R,3S,4S,5S,6R)-3,4,5-trihydroxy-6-(hydroxymethyl)-tetrahydro-2H-pyran-2-yl)methyl)-N-methyl-5′-(trifluoromethyl)biphenyl-3-carboxamide,
4′-((S)-hydroxy ((2R,3S,4S,5S,6R)-3,4,5-trihydroxy-6-(hydroxymethyl)tetrahydro-2H-pyran-2-yl)methyl)-N-methyl-3′-(trifluoromethyl)-[1′-biphenyl]-3-carboxamide,
4′-((R)-Hydroxy ((2R,3S,4S,5S,6R)-3,4,5-trihydroxy-6-(hydroxymethyl)-tetrahydro-2H-pyran-2-yl)methyl)-N,N,5′-trimethylbiphenyl-3-carboxamide,
5-Cyano-4′-((R)-hydroxy ((2R,3S,4S,5S,6R)-3,4,5-trihydroxy-6-(hydroxymethyl)-tetrahydro-2H-pyran-2-yl)methyl)-N,5′-dimethylbiphenyl-3-carboxamide,
4-Cyano-4′-((R)-hydroxy ((2R,3S,4S,5S,6R)-3,4,5-trihydroxy-6-(hydroxymethyl)-tetrahydro-2H-pyran-2-yl)methyl)-N,5′-dimethylbiphenyl-3-carboxamide,
4′-((R)-azido((2R,3S,4R,5S,6R)-3,4,5-trihydroxy-6-(hydroxymethyl)tetrahydro-2H-pyran-2-yl)methyl)-N,3′-dimethylbiphenyl-3-carboxamide,
3′-chloro-4′-((R)-hydroxy((2R,3S,4S,5S,6R)-3,4,5-trihydroxy-6-(hydroxymethyl)tetrahydro-2H-pyran-2-yl)methyl)-N-methylbiphenyl-3-carboxamide,
4′-((R)-hydroxy((2R,3S,4S,5 S,6R)-3,4,5-trihydroxy-6-(hydroxymethyl)tetrahydro-2H-pyran-2-yl)methyl)-3′-methylbiphenyl-4-carbonitrile,
(2R,3S,4S,5S,6R)-2-((R)-(4-azido-2-methylphenyl)(hydroxy)methyl)-6-(hydroxymethyl)tetrahydro-2H-pyran-3,4,5-triol,
(2R,3S,4S,5S,6R)-2-((R)-hydroxy(2-methyl-4-(4-phenyl-1H-1,2,3-triazol-1-yl)phenyl)methyl)-6-(hydroxymethyl)tetrahydro-2H-pyran-3,4,5-triol,
(2R,3S,4S,5S,6R)-2-((R)-hydroxy(2-methyl-4-(4-(pyridin-3-yl)-1H-1,2,3-triazol-1-yl)phenyl)methyl)-6-(hydroxymethyl)tetrahydro-2H-pyran-3,4,5-triol,
(2R,3S,4S,5S,6R)-2-((1R)-hydroxy(2-methyl-4-(4-(piperidin-3-yl)-1H-1,2,3-triazol-1-yl)phenyl)methyl)-6-(hydroxymethyl)tetrahydro-2H-pyran-3,4,5-triol,
4′-((R)-hydroxy((2R,3S,4S,5S,6R)-3,4,5-trihydroxy-6-(hydroxymethyl)tetrahydro-2H-pyran-2-yl)methyl)-3′-methylbiphenyl-3-carbonitrile
5-(4-((R)-hydroxy((2R,3S,4S,5S,6R)-3,4,5-trihydroxy-6-(hydroxymethyl)tetrahydro-2H-pyran-2-yl)methyl)-3-methylphenyl)nicotinic acid,
methyl 5-(4-((R)-hydroxy((2R,3S,4S,5S,6R)-3,4,5-trihydroxy-6-(hydroxymethyl)tetrahydro-2H-pyran-2-yl)methyl)-3-methylphenyl)nicotinate,
3-(1-(4-((R)-hydroxy((2R,3S,4S,5S,6R)-3,4,5-trihydroxy-6-(hydroxymethyl)tetrahydro-2H-pyran-2-yl)methyl)-3-methylphenyl)-1H-1,2,3-triazol-4-yl)benzoic acid,
(2R,3S,4S,5S,6R)-2-((R)-hydroxy(2-methyl-4-(3-methyl-1H-indazol-5-yl)phenyl)methyl)-6-(hydroxymethyl)tetrahydro-2H-pyran-3,4,5-triol,
6-(4-((R)-hydroxy((2R,3S,4S,5S,6R)-3,4,5-trihydroxy-6-(hydroxymethyl)tetrahydro-2H-pyran-2-yl)methyl)-3-methylphenyl)thieno[3,2-d]pyrimidine-2,4(1H,3H)-dione,
(2R,3S,4S,5S,6R)-2-((R)-hydroxy(2-methyl-4-(4-(pyridin-2-yl)-1H-1,2,3-triazol-1-yl)phenyl)methyl)-6-(hydroxymethyl)tetrahydro-2H-pyran-3,4,5-triol,
3-(4-((R)-hydroxy((2R,3S,4S,5S,6R)-3,4,5-trihydroxy-6-(hydroxymethyl)tetrahydro-2H-pyran-2-yl)methyl)-3-methyl-2-nitrophenoxy)benzonitrile,
5-(4-((R)-hydroxy((2R,3S,4S,5S,6R)-3,4,5-trihydroxy-6-(hydroxymethyl)tetrahydro-2H-pyran-2-yl)methyl)-3-methylphenyl)-N-isopropylnicotinamide,
(2R,3S,4S,5S,6R)-2-((R)-hydroxy(3-methyl-3′-(1H-tetrazol-5-yl)biphenyl-4-yl)methyl)-6-(hydroxymethyl)tetrahydro-2H-pyran-3,4,5-triol,
(2R,3S,4S,5S,6R)-2-((R)-(4-(1-aminoisoquinolin-5-yl)-2-methylphenyl)(hydroxy)methyl)-6-(hydroxymethyl)tetrahydro-2H-pyran-3,4,5-triol,
5-(4-((R)-hydroxy((2R,3S,4S,5S,6R)-3,4,5-trihydroxy-6-(hydroxymethyl)tetrahydro-2H-pyran-2-yl)methyl)-3-methylphenyl)isoquinolin-1 (2H)-one, and
(2R,3S,4S,5S,6R)-2-((R)-(4-(3-amino-1H-indazol-5-yl)-2-methylphenyl)(hydroxy)methyl)-6-(hydroxymethyl)tetrahydro-2H-pyran-3,4,5-triol.

17. A compound of Formula (I) according to claim 1 for use as a medicament.

18. A compound of Formula (I) according to claim 1 for use in the treatment of urinary tract infection (UTI).

19. A compound of Formula (I) according to claim 1 for use in the manufacture of a medicament for the prevention or treatment of a disease or condition ameliorated by the inhibition of FimH function or activity.

20. A pharmaceutical composition comprising a compound of Formula (I) according to claim 1 and a pharmaceutically acceptable carrier.

21. A method of inhibition of FimH function comprising contacting FimH with a compound of Formula (I) according to claim 1.

22. A method of treatment of a FimH-mediated disease comprising administering a therapeutically effective amount of a compound according to claim 1 to a patient in need thereof.

23. The method according to claim 22 wherein said disease is chosen from a bacterial infection, Crohn's disease (CD), and Inflammatory Bowel Disease (IBD).

24. A method of treatment of a FimH-mediated disease comprising the administration of:
a. a therapeutically effective amount of a compound of Formula (I) according to claim 1; and
b. another therapeutic agent.

25. A pharmaceutical composition comprising a compound of Formula (I) according to claim 1 formulated for oral (PO) administration.

26. The pharmaceutical composition as recited in claim 25, wherein said composition is chosen from a tablet and a capsule.
